(19) United States
(12) Patent Application Publication
(10) Pub. No.: US 2025/0244310 A1
(43) Pub. Date: Jul. 31, 2025

(54) METHODS OF IDENTIFICATION AND TARGETING OF MASTER KINASES IN CANCER

(71) Applicant: The Trustees of Columbia University in the City of New York, New York, NY (US)

(72) Inventors: Antonio IAVARONE, Sunny Isles Beach, FL (US); Anna LASORELLA, Sunny Isles Beach, FL (US)

(21) Appl. No.: 19/067,745

(22) Filed: Feb. 28, 2025

Related U.S. Application Data
(63) Continuation of application No. PCT/US23/73349, filed on Sep. 1, 2023.
(60) Provisional application No. 63/374,513, filed on Sep. 2, 2022.

(51) Int. Cl. G01N 33/50

(52) U.S. Cl. CPC G01N 33/5011; G01N 33/5023

(57) ABSTRACT

A method of treating cancer in a subject using a kinome and/or phosphorylome analysis approach, a SPHINKS computational analysis, or a combination thereof to target master kinases driving the cancer state.

This application is a continuation of International Application No. PCT/US23/73349, filed on Sep. 1, 2023, which claims the benefit of and priority to U.S. Provisional Patent Application No. 63/374,513, filed Sep. 2, 2022, each of which is incorporated herein by reference in their entireties.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### GOVERNMENT SUPPORT

This invention was made with government support under Grant Nos. CA101644, CA193313, CA131126, CA239721, CA178546, CA179044, CA190891, CA239698, CA253183, and NCI P30 Supplement GBM CARE-HOPE. The Government has certain rights in the invention.

### SEQUENCE LISTING

The instant application contains a Sequence Listing which has been submitted electronically in XML format and is hereby incorporated by reference in its entirety. Said XML copy, created on Feb. 21, 2025, is named 0019240_01288US2_SL.xml and is 2,745 bytes in size.

### BACKGROUND

Kinases are enzymes that catalyze the process of protein phosphorylation. Phosphorylation is an important mechanism for regulating cell function including cell proliferation, cell cycle, apoptosis, motility, growth, and many other cellular functions. Dysregulated kinases play a role in many disease states.

### SUMMARY OF THE INVENTION

In certain aspects, the subject matter described herein provides a method of treating cancer in a subject in need thereof, the method comprising: obtaining proteomic-phosphoproteomics data from one or more cells in a sample from a subject; analyzing kinome and/or phosphorylome of the one or more cells from the proteomic-phosphoproteomics data in the sample; and identifying a master kinase from the kinome and/or phosphorylome analysis in the sample. In some embodiments, the method further comprises administering to the subject a therapeutically effective amount of a pharmaceutical composition, wherein the composition modulates the master kinase.

In some embodiments, the analyzing comprises a SPHINKS computational analysis. In some embodiments, the subject has cancer. In some embodiments, the sample that the subject is a sample of cancer cells of the subject. In some embodiments, the cancer is a glioma, a pediatric glioma, a glioblastoma (GBM), an IDH wild-type GBM, a breast cancer, or a lung squamous cell carcinoma. In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the master kinase is (DNA-dependent protein kinase catalytic subunit) DNA-PKcs. In some embodiments, the composition comprises M3814 (nedisertib). In some embodiments, the composition comprises an inhibitory RNA specific for a gene encoding the master kinase. In some embodiments, the inhibitory RNA is one or more of a miRNA, a siRNA, a shRNA, or a piRNA.

In some embodiments, the composition modulates an expression of the master kinase. In some embodiments, the composition decreases the expression of the master kinase. In some embodiments, the composition modulates an activity of the master kinase. In some embodiments, the composition decreases the activity of the master kinase. In some embodiments, the composition is administered in combination with treating the subject with ionizing radiation (IR).

In some embodiments, the sample comprises a tissue sample. In some embodiments, the tissue sample is a frozen tissue sample. In some embodiments, the tissue sample is embedded in paraffin.

In some aspects, the subject matter described herein provides a method of treating cancer in a subject in need thereof, the method comprising: obtaining proteomic-phosphoproteomics data from one or more cells of a tumor sample from the subject; classifying the tumor sample into a tumor subtype; identifying a master kinase for the specific tumor subtype of the tumor sample; and administering to the subject a therapeutically effective amount of a pharmaceutical composition, wherein the composition modulates the master kinase.

In some embodiments, the cancer is a glioma, a pediatric glioma, a glioblastoma (GBM), an IDH wild-type GBM, a breast cancer, or a lung squamous cell carcinoma. In some embodiments, the tumor subtype is a glycolytic/plurimetabolic (GPM) subtype, mitochondrial (MTC) subtype, neuronal (NEU) subtype, or proliferative/progenitor (PPR) subtype. In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the master kinase is (DNA-dependent protein kinase catalytic subunit) DNA-PKcs. In some embodiments, the composition comprises M3814 (nedisertib). In some embodiments, the composition comprises an inhibitory RNA specific for a gene encoding the master kinase. In some embodiments, the inhibitory RNA is one or more of a miRNA, a siRNA, a shRNA, or a piRNA.

In some embodiments, the composition modulates an expression of the master kinase. In some embodiments, the composition decreases the expression of the master kinase. In some embodiments, the composition modulates an activity of the master kinase. In some embodiments, the composition decreases the activity of the master kinase. In some embodiments, the composition is administered in combination with treating the subject with ionizing radiation (IR).

In some embodiments, the tumor sample is a frozen tumor sample. In some embodiments, the tumor sample is embedded in paraffin. In some embodiments, the master kinase for the specific tumor subtype has been identified via SPHINKS computational analysis. In some embodiments, the tumor sample is classified into a subtype via a probabilistic classifying method.

In certain aspects, the subject matter described herein provides a method of associating a master kinase with a cancer sample from a subject, the method comprising: obtaining proteomic-phosphoproteomics data from one or more cells in the cancer sample from the subject; analyzing kinome and/or phosphorylome of the one or more cells from the proteomic-phosphoproteomics data in the cancer sample; and identifying a master kinase from the kinome and/or phosphorylome analysis in the cancer sample. In some embodiments, the method further comprises validating the master kinase. In some embodiments, the validating comprises experimentally validating the master kinase.

In certain aspects, the subject matter described herein provides a method of diagnosing a subject with a cancer that comprises a specific master kinase, the method comprising: obtaining proteomic-phosphoproteomics data from one or more cells in a cancer sample from the subject; analyzing kinome and/or phosphorylome of the one or more cells from the proteomic-phosphoproteomics data in the cancer sample; and identifying a master kinase from the kinome and/or phosphorylome analysis in the sample. In some embodiments, the method further comprises validating the master kinase. In some embodiments, the validating comprises experimentally validating the master kinase. In some embodiments, the master kinase is identified as a therapeutic target. In some embodiments, the method further comprises classifying the cancer sample into a tumor subtype. In some embodiments, the tumor subtype is a glycolytic/plurimetabolic (GPM) subtype, mitochondrial (MTC) subtype, neuronal (NEU) subtype, or proliferative/progenitor (PPR) subtype.

In certain aspects, a computational strategy is used called the SPHINKS approach. In some embodiments, the SPHINKS analysis comprises: (i) training a support vector machine (SVM) classifier with a positive data set comprising a set of known substrates of a specific kinase and a negative data set comprising a subset of randomly selected unknown interactions using kinase abundance from proteomics and substrate abundance from phosho-proteomics; (ii) computing a probability score for all the kinase-substrate pairs in the network according to the SVM classifier; (iii) repeating steps (i) and (ii) with the same positive data set and a different negative data set; (iv) performing machine learning ensemble meta-algorithm bagging to obtain an average of scores from each iteration of steps (i) and (ii); (v) defining a list of predicted kinase-substrate interactions by selecting a threshold for the average SVM score and retaining only interactions whose average score was above the selected threshold and whose Spearman correlation between protein kinase global abundance and substrate phospho-site abundance was positive; and (vi) calculating Master Kinase activity as the difference of the weighted average of the predicted substrate's abundances using the SVM score of kinase-substrate interactions as weight and the weighted average of randomly selected control substrate-set. In some embodiments, the selected threshold for the average SVM score is greater than 50% of the known interactions. In some embodiments, the set of known substrates of a specific kinase comprises validated kinase-substrate interactions from PhosphoSitePlus. In some embodiments, steps (i) and (ii) are repeated around 100 times. In some embodiments, in step (v) kinases with less than 10 interactions are removed from the list.

In certain aspects, the subject matter described herein provides a method of treating cancer in a subject in need thereof, the method comprising: administering to the subject a therapeutically effective amount of a pharmaceutical composition, wherein the composition modulates a master kinase. In some embodiments, the cancer is a glioma, a pediatric glioma, a glioblastoma (GBM), an IDH wild-type GBM, a breast cancer, or a lung squamous cell carcinoma. In some embodiments, the cancer is a glycolytic/plurimetabolic (GPM) subtype, mitochondrial (MTC) subtype, neuronal (NEU) subtype, or proliferative/progenitor (PPR) subtype.

In some embodiments, the composition comprises BJE-10676. In some embodiments, the cancer is a glycolytic/plurimetabolic (GPM) glioblastoma subtype. In some embodiments, the composition comprises M3814 (nedisertib). In some embodiments, the cancer is a proliferative/progenitor (PPR) glioblastoma subtype.

In certain aspects, the subject matter described herein provides a method of treating cancer in a subject in need thereof, the method comprising: determining, using a multi-omics approach, a tumor subtype of a cancer sample from the subject, wherein the multi-omics approach comprises analyzing kinase activity, radiomics, copy number variants (CNV), single nucleotide variants (SNV), and a gene expression profile of the cancer sample.

In some embodiments, the cancer is classified as a mitochondrial (MTC) subtype if it is associated with a plurality of high CET, low NET, enhanced PHKG2 expression, SLC45A1 del, RERE del, 1p36 del, enhanced OXPHOS activity, enhanced TCA cycle activity, and enhanced mitochondrial translation. In some embodiments, the cancer is classified as a glycolytic/plurimetabolic (GPM) subtype if it is associated with a plurality of high CET, low NET, high edema, male demographic, 40-65 years demographic, MET amp, NF1 mut/del, enhanced PKCδ, P38D, or MK-2 expression, enhanced glycolysis, enhanced lipid storage, or hypoxia.

In some embodiments, the cancer is classified as a neuronal (NEU) subtype if it is associated with a plurality of low CET, high NET, high WM invasion, low necrosis, ATRX mut, TCGA, enhanced GSK3β, PCKε, or PAK1/3 expression, enhanced neuronal differentiation, or excitatory synapses. In some embodiments, the cancer is classified as a proliferative/progenitor (PPR) subtype if it is associated with a plurality of low CET, high NET, low WM invasion, high edema, EGFR amp, CDK6 amp, enhanced DNA-PKcs, CDK1/2/6, or CHK2 activity, enhanced cell cycle activity, enhanced DNA replication, or enhanced DDR pathway activation.

In some embodiments, the method further comprises administering to the subject a therapeutically effective amount of a pharmaceutical composition. In some embodiments, the composition comprises BJE-10676. In some embodiments, the composition comprises M3814 (nedisertib). In some embodiments, the composition comprises an inhibitory RNA. In some embodiments, the inhibitory RNA is one or more of a miRNA, a siRNA, a shRNA, or a piRNA.

In certain aspects, the subject matter described herein provides a method of probabilistically classifying a cancer into a tumor subtype, the method comprising: obtaining a gene expression profile of a tumor sample; comparing the gene expression profile of the tumor sample with a gene expression profile of a set of tumors with known tumor subtypes; and correlating the gene expression profile from the RNA-Seq data with the best fitting tumor subtype.

In some embodiments, the gene expression profile of the tumor sample was obtained by RNA-Seq. In some embodiments, the cancer is a glioma, a pediatric glioma, a glioblastoma (GBM), an IDH wild-type GBM, a breast cancer, or a lung squamous cell carcinoma. In some embodiments, the tumor subtype is a glycolytic/plurimetabolic (GPM) subtype, mitochondrial (MTC) subtype, neuronal (NEU) subtype, or proliferative/progenitor (PPR) subtype. In some embodiments, the tumor sample is classified into a tumor subtype only if the difference between the correlation with the tumor subtype and other tumor subtypes is above a threshold value. In some embodiments, the threshold value is in the form of a simplicity score, wherein the simplicity score is a different between a highest fitted probability (dominant subtype) and a mean of the other subtypes (non-dominant). In some embodiments, the threshold value is a simplicity score of 0.35.

In some embodiments, the tumor sample comprises a tissue sample. In some embodiments, the tissue sample is embedded in paraffin. In some embodiments, the tumor sample is classified into a tumor subtype via an algorithm.

In certain aspects, the subject matter described herein provides a method of treating a cancer of a subject in need thereof, the method comprising: classifying a tumor sample from the subject into a tumor subtype using any one of the methods of the invention;
identifying a master kinase associated with the tumor subtype of the tumor sample; and administering to the subject a therapeutically effective amount of a pharmaceutical composition, wherein the composition modulates the master kinase.

In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the tumor subtype is the glycolytic/plurimetabolic (GPM) subtype. In some embodiments, the master kinase is (DNA-dependent protein kinase catalytic subunit) DNA-PKcs. In some embodiments, the composition comprises M3814 (nedisertib). In some embodiments, the tumor subtype is the proliferative/progenitor (PPR) subtype. In some embodiments, the composition comprises an inhibitory RNA specific for a gene encoding the master kinase. In some embodiments, the inhibitory RNA is one or more of a miRNA, a siRNA, a shRNA, or a piRNA.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A-C show proteogenomic interpretation of GBM functional subtypes. a, Heatmap showing the 150 highest scoring proteins in the ranked lists (MWW-test) of GPM, MTC, NEU and PPR GBM subtypes. Rows are proteins and columns are tumors (n=85 GBM IDH wild type). Left and top shading indicate GBM subtypes. b, Grid plot showing the NES of the highest active, not-redundant biological pathways for each GBM subtype [logit(NES)>0.58, FDR<0.005; two-sided MWW-GST]. c, Integrative heatmap showing CNVs (upper panels) and protein abundance (bottom panels) of genes with fCNprot gain (amp) or loss (del) as described in Methods. Gains/amplifications are indicated in light gray, loss/deletions are in dark gray. In each panel, tumors are ordered from left to right according to highest to lowest subtype activity NES (upper track); lower track indicates tumor classification. For each subtype, representative genes with the highest frequency of fCNprot gain (red squares) or loss (blue squares) are listed. NES, normalized enrichment score.

FIGS. 2A-F show association between demographic, imaging-based features and functional subtypes. a, Forest plots of univariate logistic regression model of age and gender association with GBM functional subtypes in the TCGA dataset (n=503 tumors). b, Forest plots of univariate logistic regression models of the association between tumor location and GBM functional subtypes in the TCGA dataset (n=88 tumors). c, Bar plots showing the proportion of necrosis and edema in the four functional subtypes of GBM from the TCGA cohort (n=63 tumors) and deep white matter invasion from a GBM cohort including 40 and 14 patients from the TCGA and REMBRANDT datasets, respectively. d, Forest plots of univariate logistic regression model for the association between contrast enhancing, non-contrast enhancing tumor or edema and GBM functional subtypes in the TCGA dataset (n=88 tumors); e, Univariate logistic regression for the same variables in d after aggregation of patients classified in the metabolic and neurodevelopmental axis. f, Unsupervised clustering on 175 quantitative radiomic features and functional subtypes of GBM). Heatmap of radiomic features with differential quantification in GBM intrinsic imaging subtypes (n=88 tumors, left panel). Upper track indicates GBM imaging subtypes identified by unsupervised clustering and lower track indicates tumor classification. Representative radiomic features for cluster 1 (enriched with PPR tumors) and cluster 4 (enriched with GPM tumors) are indicated. Right panel, association between radiomic clusters and GBM subtypes. Circles are color-coded and their size reflects the standardized residuals (χ2 test). Scale (−2 to 2) indicates positive (right hand side) to negative (left hand side) enrichment. Asterisks indicates standardized residuals higher than 1.5. For each univariate logistic regression model, log(odd-ratio) estimates (OR), 95% confidence intervals (CI) and p-values are reported. log(odds-ratio) estimates higher or lower than 0 represent positive or negative association, respectively.

FIGS. 3A-D show that GBM of the PPR subtype exhibits phospho-programs of DDR activity and replication stress and distinct sensitivity to DDR inhibition. a, DDR signaling network including the most enriched pathways and the highest abundant proteins in PPR GBM (MWW score >1.5) compared to the other subtypes [logit(NES)>1, p<0.001, two-sided MWW-GST]. b, Heatmap showing the phospho-protein abundance of biologically validated phosphorylation sites upregulated by irradiation-induced DNA damage response (DDR) and aphidicolin-induced DNA replication stress (RS). c, DDR (left panel) and RS-induced (right panel) signature score of GBM classified according to four functional subtypes. Top track, left to right represents tumors ranked by the highest to the lowest DDR or RS score. Upper panels, heatmap showing tumor subtype assignment by SNF. Each row represents a functional subtype. Bottom panels, heatmap showing for each tumor the difference between subtype-specific proteomic and transcriptomic activity. Each row represents a subtype specific activity. GPM, MTC, NEU, and PPR subtype specific scale indicates lowest to highest delta enrichment score for each subtype (Spearman's correlation between subtype specific activity and DDR/RS scores). Asterisks: *p<0.10, **p<0.05, ***p<0.001. d, Immunoblot of 4 GPM and 6 PPR PDOs analyzed using the indicated antibodies. Vinculin and β-actin is shown as loading control. *: non-specific band.

FIGS. 4A-D show that protein phosphorylation-kinase networks by SPHINKS reveal subtype-specific master kinases and signaling. a, Heatmap depicting the 70 highest significant outlier phosphorylated proteins in each functional GBM subtype (p<0.005, BlackSheep). Unsupervised clustering and biological pathways significantly enriched in outlier phosphoproteins are presented on the left; (Fisher exact test, p<0.01). b, Global kinase-substrate phosphosite interactome inferred by SPHINKS. Nodes represent kinases and substrate phosphosites and lines their interactions. Kinase families and phosphorylated amino acid residues are indicated by different colors. Node size of the kinases is proportional to the number of interacting phosphosites. Interactions indicate substrate phosphosites reported in the PhosphoSitePlus database as well as inferred novel interactions. c, Circular plot depicting the most active kinases in each GBM subtype compared with all other subtypes (effect size >0.3, p<0.01; two-sided MWW test) with the outermost circle representing the color scale of kinase activity (CHK2, CDK2, DNAPK, CDK6, CK2A1, CDK1, RAF1 are proliferative/progenitor; S6K2, IKKB, MNK1, AMPKA1, MK-2, SYK, P38D, VRK2, PKCD are glycolytic/plurimetabolic; BRAF, TTBK2, JNK3, PAK1, PAK3, PKCE, GSK3B are neuronal; PHKG2 is mitochondrial). The five predicted kinase-regulated phosphorylation sites with the highest SVM probability score are indicated and represented by black dots: SVM probability score within the dashed line: >0.95; SVM probability score between dashed and continuous line: 0.95-0.90; SVM probability score inside the continuous line: <0.90. d, Heatmaps showing kinase activity (NES), MWW protein abundance score, and MWW gene expression score of SPHINKS-MKs specific for each CPTAC-GBM subtype. Heatmaps depicting MWW gene expression score of the same kinases in single GBM cells and PDOs signify the cancer cell intrinsic expression of the top scoring kinases identified by SVM. Only values of logit(NES)>0.58 are shown.

FIGS. 5A-M show validation of dependency of GBM cells on specialized protein kinases. a, Viability curves of GPM PDOs treated with the indicated compounds or irradiation. Data are mean±s.d. of n=3-6 for compound treatment and n=8 replicates for irradiation from one representative experiment. Experiment was repeated three times with similar results. b, Viability curves of 14 GPM PDOs treated with BJE-106. Data are mean±s.d. of n=6-18 replicates for each PDO from one representative experiment. Experiment was repeated at least two times with similar results. c, Colony-forming assay using GPM PDO cells treated with the indicated concentration of BJE6-106. The bar graph shows the mean±s.d of n=3 technical replicates. *p≤0.05, **p≤0.005, two-tailed t-test, unequal variance. The experiment was repeated two times with similar results. Legend top to bottom shows order of bars from left to right. d, Western blot analysis of GPM PDO cells treated with BJE6-106 at concentration of 50 M and harvested at indicated time points using the indicated antibodies. β-actin is included as loading control. e, Immunoblot of GPM PDO cells transduced with lentivirus expressing two different sh-RNAs targeting PRKCD or the empty vector. 3-actin shown as loading control. f-g, Growth curves of two independent GPM PDOs each expressing two independent sh-RNAs targeting PRKCD or the empty vector. Data are mean±s.d from n=5 (f) and n=6 (g) biological replicate for each PDO of survival values normalized to values obtained 16 h after plating (DO); **p≤0.0001, non-target (NT) versus sh-PRKCD #1; **p≤0.0001, non-target (NT) versus sh-PRKCD #2. h, Quantification of sphere-forming assay for GPM PDO cells shown in the experiment in g. Data are mean±s.d. from one representative experiment; n=3 biological replicates (independent infections); **p≤0.0001, non-target (NT) versus sh-PRKCD #1; **p≤0.0001, non-target (NT) versus sh-PRKCD #2. Order of bars from left to right is shRNA NT, shRNA PRKCD #2, shRNA PRKCD #1. i, Rate of glucose uptake in GPM PDO cells expressing two different sh-RNAs targeting PRKCD or the empty vector. Data are mean±s.d. of one representative experiment; n=6 for sh-RNA NT, 3 for sh-PRKCD #1 and 4 for sh-PRKCD #2; *p≤0.001, non-target (NT) versus sh-PRKCD #1; *P≤0.001, non-target (NT) versus sh-PRKCD #2. Order of bars from left to right is shRNA NT, shRNA PRKCD #2, shRNA PRKCD #1. j, Concentration of triacylglycerol in GPM PDO cells expressing two different sh-RNAs for PRKCD or the empty vector. Data are mean±s.d. of one representative experiment; n=4 for sh-RNA NT, 3 for sh-PRKCD #1 and 6 for sh-PRKCD #2; *p≤0.005, non-target (NT) versus sh-PRKCD #1; **p≤0.0001, non-target (NT) versus sh-PRKCD #2. Statistical significance was established by two-tailed t-test unequal variance. Experiments were repeated twice with similar results. Order of bars from left to right is shRNA NT, shRNA PRKCD #2, shRNA PRKCD #1. k, Cell viability after irradiation minus or plus nedisertib at increasing concentration of 8 PPR and 8 GPM PDOs. Data are mean±s.d. of one representative experiment assessed by n=4 replicates for each PDO. The experiment was repeated at least two times with similar results. Order of bars from left to right is 0 nM, 185 nM, 556 nM, 1667 nM. 1, Western blot analysis of PPR PDO cells treated with either irradiation or irradiation plus nedisertib for the indicated times using the indicated antibodies. Vinculin and β-actin are shown as loading controls. m, Quantification of 7-H2AX foci per nucleus in PPR PDO cells after treatment with either irradiation (IR, 4 Gy) or irradiation plus nedisertib (556 nM) at different time intervals. At least 50 nuclei were analyzed in each experimental group. The data are presented as mean±sem; p≤0.0001, IR versus IR plus Nedisertib at 0.5, 2, 4, 6, 9 and 24 hours; NS, not significant, for control IR versus IR plus Nedisertib. Significance was established by two-tailed t-test unequal variance (Mann-Whitney test). The experiment was repeated twice with similar results. Order of bars from left to right is IR at 0, 0.5, 2, 4, 6, 9, 24 hr, IR+Nedisertib at 0, 0.5, 2, 4, 6, 9, 24 hr.

FIGS. 6A-H show that functional activities of GBM subgroups classify different cancer types and inform survival and master kinases. a, Heatmap showing the 150 highest scoring proteins (upper panel) and phospho-sites (normalized by protein abundance, bottom panel) in the ranked lists (MWW test) of the four functional subtypes of PG; rows are proteins/phospho-sites and columns are tumors (n=104). Left and top shading indicate the functional subtypes. Middle shading indicates the tumor grade. Bottom shading indicates BRAF status. Unsupervised clustering of protein/phospho-sites signatures and pathway significantly enriched in protein/phospho-site clusters are reported on the left (p<0.05, Fisher exact test). b, Association of tumor grade with the functional subtypes of PG. Bars indicate the standardized residuals from χ2 test. c, Association of BRAF status with functional subtypes of PG-LGG. Bars indicate the standardized residuals from χ2 test. d, Heatmap showing the 150 highest scoring proteins (upper panel) and phosphosites (bottom panel) of the ranked lists (MWW test) of functional subtypes in CPTAC breast cancer samples. Rows are proteins/phospho-sites and columns are tumors (n=118). Horizontal top and left tracks indicate functional subtypes; horizontal middle track indicates the NMF multi-omics classification of BRCA proposed by CPTAC (I, inclusive); horizontal lower track indicates tumor grade. Unsupervised clustering of each subtype-specific signature and pathway significantly enriched in each protein/phospho-site subcluster are reported on the left [p<0.05, Fisher exact test]. e, Enrichment of NMF-based BRCA subtypes in the four functional subtypes. Circles are color-coded and their size reflects the standardized residuals from χ2 test. Scale indicates positive to negative enrichment. f, Heatmap showing the 150 highest scoring proteins (upper panel) and phospho-sites (bottom panel) of the ranked lists (MWW-test) of functional subtypes in LSCC from the CPTAC cohort. Rows are proteins/phospho-sites and columns are tumors (n=106). Horizontal top and left tracks indicate functional subtypes; horizontal middle track indicates the NMF multi-omics classification of LSCC proposed by CPTAC; horizontal lower track indicates tumor grade. Unsupervised clustering of each subtype-specific signature and pathway significantly enriched in protein/phospho-site subcluster are reported on the left [p<0.05, Fisher exact test]. g, Enrichment of NMF-based subtypes of LSCC in the four functional subtypes. Circles are shade-coded and their size reflects the standardized residuals from χ2 test. Scale indicates positive to negative enrichment. h, Grid plot showing the top-scoring MKs common to each functional subtype of GBM, PG, BRCA and LSCC tumors. Dots are shaded according to kinase activity and their size reflect the significance of the differential activity in each group when compared to the others (MWW score >0.3, p<0.01).

FIGS. 7A-D show a probabilistic classifier for the identification of functional tumor subtypes of IDH wild type GBM. a, GBM subtype-specific ROC curves for the multinomial regression model using RNA-Seq data from frozen samples. Validation includes RNA-Seq data from TCGA (left panel), or CPTAC (right) GBM samples. b, Comparison bar plot of sensitivity, specificity, and precision in each GBM subtype of the multinomial regression model as in a. Dashed lines and corresponding values indicate the average of each performance measure (sensitivity (0.84, 0.84, respectively); specificity (0.94, 0.95, respectively); precision (0.82, 0.86, respectively) in each GBM subgroup. Each group of bars from left to right are sensitivity, specificity, and precision. c, GBM subtype-specific ROC curves for the multinomial regression model using RNA-Seq data from FFPE samples. Validation includes RNA-Seq obtained from FFPE tumor samples. d, Comparison bar plot of sensitivity, specificity, and precision in each GBM subtype of the multinomial regression model as in c. Dashed lines and corresponding values indicate the average of each performance measure (sensitivity (0.84); specificity (0.95); precision (0.86)) in each GBM subgroup. Each group of bars from left to right are sensitivity, specificity, and precision.

FIGS. 8A-F show a definition of functional subtypes of GBM by SNF and relationship to prior GBM classifiers. a, Circular plot indicating the annotation of data available for each platform and individual tumors of CPTAC-GBM cohort (n=93 IDHwt tumors). b, Integrative clustering of GBM tumors by SNF (n=89). Heatmap of patient-to-patient similarity coefficients generated by the integration of subtype-specific gene expression of the highest 50 genes in the ranked lists of the functional subtypes of 52 GBM tumors classified as anchors and fCNVs associated with the four GBM subtypes from TCGA. Light to dark scale represents low to high similarity coefficient. c, Dot plot showing the genes harboring fCNV^{prot} gain or loss and relative pathway enrichment for each GBM subtype. Dot size indicates significance of the pathway enrichment (p<0.05, Fisher exact test) and shading the log₂(FC) of the protein abundance in tumors harboring the fCNVprot alteration compared to wild type tumors (scale indicate fCNV^{prot} gain to fCNVprot loss; *=positive fCNV^{prot} gain to fCNVprot loss). d-e, Chord diagram of GBM subtype assignment of the indicated classifiers in each individual tumor from TCGA (n=199 tumors) (d) and CPTAC (n=83 tumors) (e) datasets. f, Chord diagram of GBM subtype assignment according to the indicated classifiers in each individual tumor from the CPTAC dataset (n=85 tumors).

FIGS. 9A-C show an association between fCNV status of GBM driver genes and pathway-based subtypes. a, Forest plots showing univariate logistic regression models for the association between fCNV amplification/mutation status of GBM driver oncogenes and subtype transcriptomic activity (top value of each pair of values) or abundance of protein of the corresponding gene (bottom value of each pair of values) in the CPTAC-GBM cohort (n=84 tumors). b, FGFR3-TACC3 fusion analysis was performed using a cohort of 178 GBM profiled by FFPE tissue RNA-Seq. c, Forest plots showing univariate logistic regression models for the association between fCNV deletion/mutation status in GBM tumor suppressor genes and subtype transcriptomic activity (top value of each pair of values) or protein abundance of the corresponding gene (bottom value of each pair of values) in the same cohort as in a. For each model, log(odds-ratio) estimates (OR), 95% confidence intervals (CI), and p-values are reported; log(odds-ratio) estimates higher or lower than 0 represent positive or negative association, respectively. For tumor suppressor genes, subtype activity values (NES) were multiplied by −1 for visualization purpose.

FIGS. 10A-F show a multiplatform validation of the metabolic axis of the GBM subtypes. a, Comparative analysis of the interactome network including intermediate metabolites and enzymes of the indicated metabolic activities in GPM versus MTC tumors (GPM: n=16; MTC: n=10 for metabolites; GPM: n=22; MTC: n=12 for proteins). Scale indicates metabolite/protein increase to decrease in GPM versus MTC tumors; [glycolytic intermediates: logit(NES)=1.76, p=0.0007, mitochondrial intermediates: logit(NES)=−1.65, p=0.018; glycolytic proteins: logit(NES)=1.27, p=0.017, mitochondrial proteins: logit(NES)=−1.19, p=5.93e-13; two-sided MWW-GST]. b-d, Enrichment analysis of b, lipid subclasses and c, LION terms, grouped according to cellular components and d, lipid functions. Lipid subclasses and LION terms significantly enriched in at least one GBM subtype are reported (log odd-ratio>0 and p<0.05, Fisher exact test). Circles are shade-coded and their size reflect the log-odd ratio. 1980 Asterisks: *p<0.05, **p<0.005, ***p<0.001. e, Heatmap showing unsupervised clustering of metabolic proteins differentially expressed between MTC and GPM tumors [log 2(FC)>0.3, p<0.05; two-sided MWW test]. Biological pathways significantly enriched in metabolic proteins are reported on the right (log odd-ratio>0 and p<0.05, Fisher exact test). f, Heatmap depicting the outlier fraction of acetylated metabolic protein in GPM and MTC tumors (p<0.05, BlackSheep). Representative outlier acetylated proteins are listed on the left according to decreasing p-value. Biological pathways significantly enriched in outlier acetylated proteins are reported on the right (Fisher exact test, p<0.0005).

FIGS. 11A-G show that protein acetylation defines distinct PPR subpopulations. a, Heatmap showing unsupervised clustering of GBM tumors using the most variable nuclear protein acetyl-sites (n=320). b, Association between acetyl-site clusters and functional subtypes of GBM. Circles are shade-coded and their size reflects the standardized residuals from χ2 test. Scale indicates positive to negative enrichment. Asterisks indicates standardized residuals higher than 2. c, Heatmap showing unsupervised clustering of differential acetylated nuclear proteins in PPR tumors with high- (PPR in cluster 2 in c) and low- (PPR in cluster 3 in c) acetylation of nuclear proteins [log₂(FC)>0.3, p<0.001; two-sided MWW test]. d, Box plots of PPR activity calculated from the transcriptome (left panel) or global proteome (right panel) in PPR GBM with low and high acetylation (two-sided MWW test). e, Box plots of stemness activity calculated from the transcriptome (left panel) or global proteome (right panel) in PPR GBM with low and high acetylation (two-sided MWW test). Box plots span the first to third quartiles and whiskers show the 1.5×interquartile range. f, Scatterplot comparing global protein and acetyl-site abundance between high- and low-acetylated PPR GBM. The x axis indicates protein log₂(FC) multiplied by −log 10(p-value). The y axis indicates acetyl-site log₂(FC) multiplied by −log 10(p-value). The horizontal and vertical lines denote the cut-off of log₂(FC)=0.5 multiplied by −log 10(p=0.05). g, GO over representation analysis of acetylated proteins in panel g using gProfiler (FDR<0.05).

FIGS. 12A-F show a computational strategy for the identification of MKs in functional GBM subtypes and benchmarking of SPHINKS approach. a, The approach for the reconstruction of an unbiased kinome network combines SVM classifiers trained on different instances of the negative set as follows: (step i) train SVM classifier on validated kinase-substrate interactions from PhosphoSitePlus (positive training set) and a subset of randomly selected unknown interactions (dotted arrow, negative set) using kinase abundance from proteomics and substrate abundance from phosho-proteomics; (step ii) compute a score for all the kinase-substrate pairs in the network according to the SVM classifier; (step iii) perform machine learning ensemble meta-algorithm bagging and obtain the average of scores from each iteration; (step iv) define the list of predicted kinase-substrate interactions by first selecting a threshold for the average SVM score (score >50% of the known interactions) and retaining only interactions whose average score was above the selected threshold and whose Spearman correlation between protein kinase global abundance and substrate phospho-site abundance was positive; (step v) calculate MKs activity, the difference of two terms, the weighted average of the predicted substrate's abundances using the SVM score of kinase-substrate interactions as weight (left), and the weighted average of randomly selected control substrate-set (right). b, ROC curves of the predictions of the kinase-phospho-substrate interaction by SPHINKS derived from simulated phospho-proteomic matrix with different rates of missing values. The top-left side of plot was magnified for accurate visualization of the curves. c, ROC curves of the predictions of the kinase-phospho-substrate interaction by SPHINKS for each of the 10 cross-validation iterations of experimentally validated kinase-phospho-substrate interactions from PhosphoSitePlus. d, Boxplots of the average kinase A-activity (percentage) from unperturbed versus 100 networks perturbed with random phospho-sites interactions for each kinase replacing true interactions in the network (p=5%, 10%, 15%, 20%, 50%). In the upper plot, each dot represents the average Δ-activity for each kinase across all runs at each perturbation percentage; in the lower plot, each dot represents the average Δ-activity for each run across all kinases at each ratio of perturbation. e, Kinase-substrate interactome from SVM semi-supervised method highlighting MKs for each functional subtype indicated by master kinases in GPM (MK-2, P38D, AMPKA, PKCD), MTC (PHKG2), NEU (GSK3B, PAK1), and PPR (CDK2, CK2A1, DNAPK, CDK1), respectively; effect size >0.3, p<0.01; two-sided MWW test. Nodes represent kinases and substrates, and lines their interactions. Grey nodes are subtype non-specific kinases; tiny nodes are kinase-targeted phospho-sites substrates. Lines indicate kinase-phosphosite interactions from PhosphoSitePlus and novel kinase-substrate interactions inferred by the SVM approach. F, MKs significantly active in each functional GBM subtype using the approach in A were mapped onto the human kinome tree. GPM, NEU, MTC, and PPR, are shown in shades ranging from dark to light gray, respectively. Exemplary kinases for each subtype are labelled. The size of the circles is proportional to the kinase activity.

FIGS. 13A-B show benchmarking of SPHINKS against previously published kinase substrate inference methods. a, Barplot showing the probability of correctly identifying upregulated or downregulated kinases by the analysis of the “top-10-hit” using the indicated inference methods. Legend top to bottom shows order of bars from left to right. b, Barplot of the differential rank (A-rank) of activity between SPHINKS and the indicated inference methods for the kinases significantly active in each GBM subtype by SPHINKS and common to the networks of all five approaches. Kinases are ordered according to the rank of activity by SPHINKS. Legend top to bottom shows order of bars from left to right in each group of bars.

FIGS. 14A-C show global and phospho-proteomics events in insulin receptor/IGF-PKCδ pathway in GPM GBM. a, Signaling network highlighting the molecules and proteins involved in IGF-I/insulin signaling of in GPM GBM tumors. Scale in outlined shapes indicates the MWW score derived from the proteomic ranked list of GPM tumors when compared to the others. Scale in smaller dots indicates the MWW score derived from the phospho-site ranked list of GPM subtype when compared to the others. Unshaded molecules are proteins not profiled or whose abundance was not significantly higher in GPM when compared to the other subtypes. b-c, Western blot analysis of GPM PDOs incubated with b, IGF-I (10 ng/ml), IGF-II (10 ng/ml) and c, insulin (100 ng/ml) for the indicated times using the indicated antibodies. GAPDH is shown as a loading control. Each of these experiments was repeated independently two times with similar results.

FIGS. 15A-B show that enrichment of DDR and RS phosphoproteins is a specific feature of PPR GBM. a, Viability curves of 8 PPR and 8 GPM PDOs treated with increasing concentration of Nedisertib. Data are mean±s.d. of n=4 replicates for each PDO from one representative experiment. Experiments were repeated two times with similar results. b, Quantification of clonogenic assay for 2 PPR and 2 GPM PDOs treated with either irradiation or irradiation plus Nedisertib. Data are mean±s.d. from triplicate cultures of 96 well plates for each point.

FIGS. 16A-C show proteomics characterization and clinical outcome of PG stratified according to functional subtypes. a-b, Heatmap showing the median abundance of the 150 highest scoring proteins of the ranked lists (MWW test) of the four functional subtypes in a, low-grade and b, high-grade pediatric glioma. Rows are proteins and columns are functional subtypes (n=82 low grade; n=22 high grade). Left and top tracks indicate functional subtypes. Unsupervised clustering was performed for each subtype-specific protein signature. For each subtype, biological pathways significantly enriched by each gene subcluster are reported on the left (p<0.05, Fisher exact test). c, Kaplan-Meier curves of pediatric glioma patients (n=94) stratified by SNF combining gene and protein signatures obtained from the functional GBM subtypes. Patients in the PPR subgroup exhibit significantly worse survival (log-rank test).

FIGS. 17A-F show functional classification of BRCA and LSCC and prognostic implications. a-b, Heatmap showing the 150 highest scoring genes of the ranked lists (MWW 2083 test) of the four functional subtypes obtained from tumors classified in a, TCGA- (n=810) and b, METABRIC-BRCA (n=1,088) datasets. Rows are genes and columns are tumors. Horizontal top and left tracks indicate functional subtypes; horizontal middle track indicates PAM50 classification of BRCA by TCGA; horizontal lower track indicates tumor grade. Unsupervised clustering was performed for each subtype-specific gene signature. Biological pathways significantly enriched by each gene subcluster are reported on the left [p<0.05, Fisher exact test]. c, Kaplan-Meier curves and log-rank test analysis of 1,897 BRCA patients from the combined TCGA (n=809) and METABRIC datasets (n=1,088), stratified according to the four functional subclasses. d, Heatmap showing the 150 highest scoring genes of the ranked lists (MWW test) of the four functional subtypes in lung squamous cell carcinoma from TCGA database (n=360). Rows are genes and columns are tumors. Horizontal top and left tracks indicate functional subtypes; horizontal lower track indicates tumor grade. Unsupervised clustering was performed for each subtype-specific gene signature. For each subtype, biological pathways significantly enriched by each gene subcluster are reported on the left (p<0.05, Fisher exact test). e, Kaplan-Meier curves and log-rank test analysis of 356 patients with LUSC from the TCGA dataset stratified according to the four functional subclasses. f, Mitochondrial activity (NES) and menadione survival ratio (log 2) for 26 BRCA (upper plot) and 71 LSCC (lower plot) cell lines from DepMap. Upper track, functional classification; middle track, mitochondrial activity; lower track, menadione survival ratio. Survival ratio: difference between mitochondrial cell lines versus the others; log₂(FC)=−1.31, p=0.008 for BRCA; log 2(FC)=−0.63, p=0.076 for LUSC; two-sided Student t-test, unequal variance.

FIGS. 18A-B show clinical-grade probabilistic tool for the classification of IDH wild type GBM. a, Schematics of the approach for calculating the probability of a GBM sample of belonging to one of the four defined functional subtypes. The Agilent expression data of 506 tumors from the TCGA cohort of IDH wild type GBM were classified into one of the four functional subtypes (top left). The standardized expression of all the genes from the subtype-specific gene signatures (bottom left) was used to train a multinomial regression model with lasso penalty using glmnet R package (middle part). Each sample (input) was used to build a multi-class logistic regression model that returns four probabilities P_{i,k}, one for each functional GBM subtype. We classified a tumor into one subtype if the fitted probability of the particular subtype was the highest (Pₖhigh) and the sample showed a simplicity score (SS) above a defined threshold (δ). Tumors that did not comply with the defined thresholds remained unclassified. b, Consensus clustering generated from the 178 FFPE GBM samples using the expression of the 200 genes from the FFPE-specific gene signatures. Columns and rows represent FFPE samples. Color bar on the top defines four subgroups according consensus clustering. Track at bottom indicates the functional classification of the corresponding 45 matched frozen tumors. Yellow-to-blue scale indicates low to high similarity.

FIG. 19 show schematic multi-omics and clinical modules characterizing the functional subtypes of GBM. Functional activities, genetic alterations, MKs, clinical characteristics, radiomic features, and therapeutic vulnerability compose modules that distinguish each functional subtype. GBM driver genes in each module recapitulate the functional hallmark of each subtype (e.g., CDK6 amplification/CDKN2A deletion for the PPR proliferation/stemness features; MET amplification/NF1 deletion for glycolysis/RAS pathway activation in GPM GBM; FGFR3-TACC3 fusion for mitochondrial activation in MTC tumors). GPM is the only subtype that significantly associates with a specific gender (male) and age group (40-65 years). GPM and MTC subtypes exhibit positive correlation with frontal/parietal and temporal tumor location, respectively. GPM, PPR and NEU are linked with radiologic features that are compatible with the biological traits of these subgroups (CET, NET and DWM invasion, respectively). In agreement with the enhanced OXPHOS and MK activity of PKCδ and DNA-PKcs in MTC, GPM, and PPR, respectively, these subtypes are distinctly sensitive to mitochondrial, PKCδ and DNA-PKcs inhibitors. CET: Contrast enhancing tumor, NET: Non-contrast enhancing tumor, DWM: deep white matter).

### DETAILED DESCRIPTION OF THE INVENTION

Kinases are enzymes that catalyze protein phosphorylation. Phosphorylation is an important mechanism for regulating cell proliferation, cell cycle, apoptosis, motility, growth, and many other cellular functions. Perturbed kinases are oncogenic and can be crucial for cancer cell genesis and proliferation; therefore, understanding the specific kinase proteins responsible for the pathogenesis of various cancers can lead to more efficient drug-target discovery and development. In some embodiments, the subject matter described herein relates to a computational pipeline that can identify major kinases in different cancer subtypes. In some embodiments, the subject matter described herein has been experimentally validated and extends the current possibilities for precision cancer medicine. Cicenas J, Zalyte E, Bairoch A, Gaudet P. Kinases and Cancer. Cancers. 2018 March; 10(3): 63; Bhullar K S, Lagaron N O, McGowan E M, Parmar I, Jha A, Hubbard B P, Rupasinghe H P V. Kinase-targeted cancer therapies: progress, challenges and future directions. Molecular Cancer. 2018 February; 17(48).

In some embodiments, the subject matter described herein relates to a computational method referred to as Substrate PHosphosite based Inference for Network of KinaseS (SPHINKS). In some embodiments, the subject method described herein relates to generating an unbiased kinome-phosphosite network. In some embodiments, the subject method described herein relates to extracting Master Kinases (MKs) driving tumor subtypes.

In some embodiments, the SPHINKS algorithm exhibits a marked stability against multiple levels of perturbations of the input dataset. In some embodiments, the SPHINKS algorithm performs better in comparison to any other methods of kinase-phosphosite inference (e.g., but not limited to KSEA and KEA3). In some embodiments, protein kinase C delta (PKCδ) has been validated as the MK that sustains cell growth and tumor cell identity of the glycolytic/plurimetabolic (GPM) functional glioblastoma (GBM) subtype. In some embodiments, DNA-dependent protein kinase catalytic subunit (DNA-PKcs) has been validated as the MK that sustains cell growth and tumor cell identity of the proliferative/progenitor (PPR) functional glioblastoma (GBM) subtype. In some embodiments, PKCδ and DNA-PKcs were confirmed as MKs in GPM and PPR tumors from pediatric glioma (PG), breast carcinoma (BRCA) and lung squamous cell carcinoma (LSCC) cohorts classified according to the four functional classes that recapitulate the metabolic and proliferation tumor cell states. In some embodiments, the subject matter described herein relates to a method which can be applied to any cancer type to first identify the MK associated with individual tumors and then allow targeted therapies with specific kinase inhibitors, therefore greatly extending the current possibilities for precision cancer medicine. In some embodiments, the subject matter described herein relates to the development of a clinical-grade probabilistic classification tool for GBM that exhibits optimal performance in both frozen and formalin-fixed, paraffin-embedded tumor tissue for application in cancer clinical pathology.

In some embodiments, the subject matter described here is useful as a development tool for cancer treatment. In some embodiments, the subject matter described here is useful as research model for probing kinase function in cancers. In some embodiments, the subject matter described here is useful as a classifier for identifying common denominator proteins in various tissue types.

### Methods of Treating Cancer

In certain aspects, the subject matter described herein provides a method of treating cancer in a subject in need thereof, the method comprising: obtaining proteomic-phosphoproteomics data from one or more cells in a sample from a subject; analyzing kinome and/or phosphorylome of the one or more cells from the proteomic-phosphoproteomics data in the sample; and identifying a master kinase from the kinome and/or phosphorylome analysis in the sample. In some embodiments, the method further comprises administering to the subject a therapeutically effective amount of a pharmaceutical composition, wherein the composition modulates the master kinase.

In some embodiments, the analyzing comprises a SPHINKS computational analysis. In some embodiments, the cancer is a glioma, a pediatric glioma, a glioblastoma (GBM), an IDH wild-type GBM, a breast cancer, or a lung squamous cell carcinoma. In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the master kinase is (DNA-dependent protein kinase catalytic subunit) DNA-PKcs. In some embodiments, the composition comprises M3814 (nedisertib). In some embodiments, the composition comprises an inhibitory RNA specific for a gene encoding the master kinase. In some embodiments, the inhibitory RNA is one or more of a miRNA, a siRNA, a shRNA, or a piRNA.

In some embodiments, the composition modulates an expression of the master kinase. In some embodiments, the composition decreases the expression of the mater kinase. In some embodiments, the composition modulates an activity of the master kinase. In some embodiments, the composition decreases the activity of the master kinase. In some embodiments, the composition is administered in combination with treating the subject with ionizing radiation (IR).

In some embodiments, the sample comprises a tissue sample. In some embodiments, the tissue sample is a frozen tissue sample. In some embodiments, the tissue sample is embedded in paraffin.

In some aspects, the subject matter described herein provides a method of treating cancer in a subject in need thereof, the method comprising: obtaining proteomic-phosphoproteomics data from one or more cells of a tumor sample from the subject; classifying the tumor sample into a tumor subtype; identifying a master kinase for the specific tumor subtype of the tumor sample; and administering to the subject a therapeutically effective amount of a pharmaceutical composition, wherein the composition modulates the master kinase.

In some embodiments, the cancer is a glioma, a pediatric glioma, a glioblastoma (GBM), an IDH wild-type GBM, a breast cancer, or a lung squamous cell carcinoma. In some embodiments, the tumor subtype is a glycolytic/plurimetabolic (GPM) subtype, mitochondrial (MTC) subtype, neuronal (NEU) subtype, or proliferative/progenitor (PPR) subtype. In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the master kinase is (DNA-dependent protein kinase catalytic subunit) DNA-PKcs. In some embodiments, the composition comprises M3814 (nedisertib). In some embodiments, the composition comprises an inhibitory RNA specific for a gene encoding the master kinase. In some embodiments, the inhibitory RNA is one or more of a miRNA, a siRNA, a shRNA, or a piRNA.

In some embodiments, the composition modulates an expression of the master kinase. In some embodiments, the composition decreases the expression of the mater kinase. In some embodiments, the composition modulates an activity of the master kinase. In some embodiments, the composition decreases the activity of the master kinase. In some embodiments, the composition is administered in combination with treating the subject with ionizing radiation (IR).

In some embodiments, the tumor sample is a frozen tumor sample. In some embodiments, the tumor sample is embedded in paraffin. In some embodiments, the master kinase for the specific tumor subtype has been identified via SPHINKS computational analysis. In some embodiments, the tumor sample is classified into a subtype via a probabilistic classifying method.

In certain aspects, the subject matter described herein provides a method of treating cancer in a subject in need thereof, the method comprising: administering to the subject a therapeutically effective amount of a pharmaceutical composition, wherein the composition modulates a master kinase. In some embodiments, the cancer is a glioma, a pediatric glioma, a glioblastoma (GBM), an IDH wild-type GBM, a breast cancer, or a lung squamous cell carcinoma. In some embodiments, the cancer is a glycolytic/plurimetabolic (GPM) subtype, mitochondrial (MTC) subtype, neuronal (NEU) subtype, or proliferative/progenitor (PPR) subtype.

In some embodiments, the composition comprises BJE-10676. In some embodiments, the cancer is a glycolytic/plurimetabolic (GPM) glioblastoma subtype. In some embodiments, the composition comprises M3814 (nedisertib). In some embodiments, the cancer is a proliferative/progenitor (PPR) glioblastoma subtype.

In certain aspects, the subject matter described herein provides a method of treating cancer in a subject in need thereof, the method comprising: determining, using a multi-omics approach, a tumor subtype of a cancer sample from the subject, wherein the multi-omics approach comprises analyzing kinase activity, radiomics, copy number variants (CNV), single nucleotide variants (SNV), and a gene expression profile of the cancer sample.

In some embodiments, the cancer is classified as a mitochondrial (MTC) subtype if it is associated with a plurality of high CET, low NET, enhanced PHKG2 expression, SLC45A1 del, RERE del, 1p36 del, enhanced OXPHOS activity, enhanced TCA cycle activity, and enhanced mitochondrial translation. In some embodiments, the cancer is classified as a glycolytic/plurimetabolic (GPM) subtype if it is associated with a plurality of high CET, low NET, high edema, male demographic, 40-65 years demographic, MET amp, NF1 mut/del, enhanced PKCδ, P38D, or MK-2 expression, enhanced glycolysis, enhanced lipid storage, or hypoxia.

In some embodiments, the cancer is classified as a neuronal (NEU) subtype if it is associated with a plurality of low CET, high NET, high WM invasion, low necrosis, ATRX mut, TCGA, enhanced GSK3β, PCKε, or PAK1/3 expression, enhanced neuronal differentiation, or excitatory synapses. In some embodiments, the cancer is classified as a proliferative/progenitor (PPR) subtype if it is associated with a plurality of low CET, high NET, low WM invasion, high edema, EGFR amp, CDK6 amp, enhanced DNA-PKcs, CDK1/2/6, or CHK2 activity, enhanced cell cycle activity, enhanced DNA replication, or enhanced DDR pathway activation.

In some embodiments, the method further comprises administering to the subject a therapeutically effective amount of a pharmaceutical composition. In some embodiments, the composition comprises BJE-10676.

In certain aspects, described herein are nucleic acids encoding RNAs of interest, which includes, but is not limited to an interfering RNA (iRNA), and variants thereof, that can silence a target gene, such as the master kinase. An iRNA can down-regulate the expression of a target gene, e.g., a master kinase. An iRNA may act by one or more of a number of mechanisms, including post-transcriptional cleavage of a target mRNA sometimes referred to in the art as RNAi, or pre-transcriptional or pre-translational mechanisms. An iRNA can be a double stranded (ds) iRNA. A ds iRNA includes more than one, and in certain embodiments two, strands in which interchain hybridization can form a region of duplex structure. A strand refers to a contiguous sequence of nucleotides (including non-naturally occurring or modified nucleotides). At least one strand can include a region which is sufficiently complementary to a target RNA. Such strand is termed the antisense strand. A second strand comprised in the dsRNA which comprises a region complementary to the antisense strand is termed the sense strand. However, a ds iRNA can also be formed from a single RNA molecule which is, at least partly; self-complementary, forming, e.g., a hairpin or panhandle structure, including a duplex region. In such case, the term strand refers to one of the regions of the RNA molecule that is complementary to another region of the same RNA molecule. Nonlimiting examples of inhibitory RNA include miRNA, siRNA, shRNA, and piRNA.

iRNA as described herein, including ds iRNA and siRNA, can mediate silencing of a gene, e.g., by RNA degradation. In certain embodiments, the gene to be silenced is a master kinase.

In certain embodiments, the oligonucleotide of interest is a guide RNA (gRNA) or single guide RNA (sgRNA). The CRISPR/Cas9 gene editing technique promotes a new human gene therapy strategy by correcting a defect gene at pre-chosen sites without altering the endogenous regulation of the target gene. This system consists of two key components: Cas9 protein and a guide RNA, e.g., a single guide RNA (sgRNA), as well as a correction template when needed. sgRNA contains two components: a 17-20 nucleotide sequence termed crispr RNA that is complementary to the target DNA region, and a tracr RNA that serves as the binding scaffold for a Cas nuclease. The sgRNA recognizes the target DNA and guides the Cas9 nuclease to the region for editing.

Therapeutically effective amount refers to an amount that is effective for preventing, ameliorating, treating or delaying the onset of a disease or condition. The pharmaceutical compositions of the inventions can be administered to any animal that can experience the beneficial effects of the agents of the invention. Such animals include humans and non-humans.

Routes of administration and dosages of effective amounts of the pharmaceutical compositions are also disclosed. The agents of the present invention can be administered in combination with other pharmaceutical agents in a variety of protocols for effective treatment of disease.

Pharmaceutical compositions are administered to a subject in a manner known in the art. The dosage administered will be dependent upon the age, health, and weight of the recipient, kind of concurrent treatment, if any, frequency of treatment, and the nature of the effect desired. One may administer the pharmaceutical compositions in a local rather than systemic manner, for example, via injection of directly into the desired target site, often in a depot or sustained release formulation.

One of ordinary skill in the art will appreciate that a method of administering pharmaceutically effective amounts of the pharmaceutical compositions to a patient in need thereof, can be determined empirically, or by standards currently recognized in the medical arts. The pharmaceutical compositions can be administered to a patient as pharmaceutical compositions in combination with one or more pharmaceutically acceptable excipients. It will be understood that, when administered to a human patient, the total daily usage of the agents of the pharmaceutical compositions will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

In some embodiments, the pharmaceutical composition used for methods of treatment described herein comprises any of the FDA-approved kinase inhibitors as described in Roskoski, R., Jr. Properties of FDA-approved small molecule protein kinase inhibitors: A 2021 update. Pharmacol Res 165, 105463 (2021), the content of which is hereby incorporated by reference it is entirety.

Methods of Associating a Master Kinase with Cancer

In certain aspects, the subject matter described herein provides a method of associating a master kinase with a cancer sample from a subject, the method comprising: obtaining proteomic-phosphoproteomics data from one or more cells in the cancer sample from the subject; analyzing kinome and/or phosphorylome of the one or more cells from the proteomic-phosphoproteomics data in the cancer sample; and identifying a master kinase from the kinome and/or phosphorylome analysis in the sample. In some embodiments, the method further comprises validating the master kinase. In some embodiments, the validating comprises experimentally validating the master kinase. In some embodiments, the method further comprises classifying the cancer sample into a tumor subtype. In some embodiments, the tumor subtype is a glycolytic/plurimetabolic (GPM) subtype, mitochondrial (MTC) subtype, neuronal (NEU) subtype, or proliferative/progenitor (PPR) subtype.

Methods of Probabilistically Classifying a Cancer into a Tumor Subtype Associated Methods of Treatment

In certain aspects, the subject matter described herein provides a method of probabilistically classifying a cancer into a tumor subtype, the method comprising: obtaining a gene expression profile of a tumor sample; comparing the gene expression profile of the tumor sample with a gene expression profile of a set of tumors with known tumor subtypes; and correlating the gene expression profile from the RNA-Seq data with the best fitting tumor subtype.

In some embodiments, the gene expression profile of the tumor sample was obtained by RNA-Seq. In some embodiments, the cancer is a glioma, a pediatric glioma, a glioblastoma (GBM), an IDH wild-type GBM, a breast cancer, or a lung squamous cell carcinoma. In some embodiments, the tumor subtype is a glycolytic/plurimetabolic (GPM) subtype, mitochondrial (MTC) subtype, neuronal (NEU) subtype, or proliferative/progenitor (PPR) subtype. In some embodiments, the tumor sample is classified into a tumor subtype only if the difference between the correlation with the tumor subtype and other tumor subtypes is above a threshold value. In some embodiments, the threshold value is in the form of a simplicity score, wherein the simplicity score is a different between a highest fitted probability (dominant subtype) and a mean of the other subtypes (non-dominant). In some embodiments, the threshold value is a simplicity score of 0.35.

In some embodiments, the tumor sample comprises a tissue sample. In some embodiments, the tissue sample is embedded in paraffin. In some embodiments, the tumor sample is classified into a tumor subtype via an algorithm.

In certain aspects, the subject matter described herein provides a method of treating a cancer of a subject in need thereof, the method comprising: classifying a tumor sample from the subject into a tumor subtype using any one of the methods of the invention; identifying a master kinase associated with the tumor subtype of the tumor sample; and administering to the subject a therapeutically effective amount of a pharmaceutical composition, wherein the composition modulates the master kinase.

In some embodiments, the master kinase is a phosphatidylinositol 3-kinase related kinase. In some embodiments, the master kinase is (Protein Kinase C delta) PKCδ. In some embodiments, the composition comprises BJE-10676. In some embodiments, the tumor subtype is the glycolytic/plurimetabolic (GPM) subtype. In some embodiments, the master kinase is (DNA-dependent protein kinase catalytic subunit) DNA-PKcs. In some embodiments, the composition comprises M3814 (nedisertib). In some embodiments, the tumor subtype is the proliferative/progenitor (PPR) subtype. In some embodiments, the composition comprises an inhibitory RNA specific for a gene encoding the master kinase. In some embodiments, the inhibitory RNA is one or more of a miRNA, a siRNA, a shRNA, or a piRNA.

### Methods of Diagnosing Cancer by Master Kinase

In certain aspects, the subject matter described herein provides a method of diagnosing a subject with a cancer that comprises a specific master kinase, the method comprising: obtaining proteomic-phosphoproteomics data from one or more cells in a cancer sample from the subject; analyzing kinome and/or phosphorylome of the one or more cells from the proteomic-phosphoproteomics data in the cancer sample; and identifying a master kinase from the kinome and/or phosphorylome analysis in the sample. In some embodiments, the method further comprises validating the master kinase. In some embodiments, the validating comprises experimentally validating the master kinase. In some embodiments, the master kinase is identified as a therapeutic target. In some embodiments, the method further comprises classifying the cancer sample into a tumor subtype. In some embodiments, the tumor subtype is a glycolytic/plurimetabolic (GPM) subtype, mitochondrial (MTC) subtype, neuronal (NEU) subtype, or proliferative/progenitor (PPR) subtype.

### SPHINKS Computational Analysis and Identification of MKs

In certain aspects, a computational strategy is used called the SPHINKS approach. In some embodiments, the SPHINKS analysis comprises: (i) training a support vector machine (SVM) classifier with a positive data set comprising a set of known substrates of a specific kinase and a negative data set comprising a subset of randomly selected unknown interactions using kinase abundance from proteomics and substrate abundance from phosho-proteomics; (ii) computing a probability score for all the kinase-substrate pairs in the network according to the SVM classifier; (iii) repeating steps (i) and (ii) with the same positive data set and a different negative data set; (iv) performing machine learning ensemble meta-algorithm bagging to obtain an average of scores from each iteration of steps (i) and (ii); (v) defining a list of predicted kinase-substrate interactions by selecting a threshold for the average SVM score and retaining only interactions whose average score was above the selected threshold and whose Spearman correlation between protein kinase global abundance and substrate phospho-site abundance was positive; and (vi) calculating Master Kinase activity as the difference of the weighted average of the predicted substrate's abundances using the SVM score of kinase-substrate interactions as weight and the weighted average of randomly selected control substrate-set. In some embodiments, the selected threshold for the average SVM score is greater than 50% of the known interactions. In some embodiments, the set of known substrates of a specific kinase comprises validated kinase-substrate interactions from PhosphoSitePlus. In some embodiments, steps (i) and (ii) are repeated around 100 times. In some embodiments, in step (v) kinases with less than 10 interactions are removed from the list.

### EXAMPLES

### Example 1—Integrative Multi-Omics Networks Identify Master Kinases of Glioblastoma Subtypes and Guide Targeted Cancer Therapy

Despite producing a panoply of potential cancer-specific targets, the proteogenomic characterization of human tumors has yet to demonstrate value for precision cancer medicine. The absence of clinically useful classifiers further hampered translation of proteogenomic information to better diagnose and treat cancer patients. In the case of the brain tumor glioblastoma (GBM), such challenges are exacerbated by tumor heterogeneity and broad therapeutic resistance. Integrative multi-omics using a machine learning-based proteomics/phosphoproteomics network identified Master Kinases (MKs) responsible for effecting key phenotypic hallmarks of the GBM subtypes that we previously characterized via pathway analysis of single cell RNAseq. The experimental follow-up in subtype-matched GBM patients derived organoid models validated Protein Kinase C delta (PKCS) and DNA-dependent protein kinase catalytic subunit (DNA-PKcs) as MKs of the Glycolytic/Plurimetabolic and Proliferative/Progenitor subtypes of GBM, respectively. Genetic and pharmacological inhibition qualified the two kinases as potent and actionable GBM subtype-specific therapeutic targets. Functional subtypes of GBM were associated with clinical and radiomics features, orthogonally validated by inspection of proteomics, phosphoproteomics, metabolomics, lipidomics and acetylomics and recapitulated in pediatric glioma, breast and lung squamous cell carcinoma, including the subtype specificity of the association of PKCδ and DNA-PKcs. To provide rapid translation of the classifier and the validated targets for precision medicine in GBM, we developed a probabilistic classification tool, that requires limited transcriptomic features and optimally performs with RNA extracted from either frozen or paraffin embedded tissues. The algorithm can be used in retrospective studies to evaluate the association of therapeutic response with GBM subtypes or as tool for patient selection in prospective clinical trials.

The classification systems of malignant tumors have evolved in the past 15 years under the pressure of the mounting molecular and genetic data and remain an active area of cancer research. The need for more accurate classifiers derives from the urgency of precision oncology and drug development targeting homogeneous tumor subsets^{1,2}. In keeping with the advances in technology and knowledge, it is recognized that while genomics offers a comprehensive view of the genetic makeup of individual tumors, the integration of genomics, protein profiling and regulation through post-translational modifications can deliver a deeper understanding of tumor biology and recognize similarity patterns within individual tumor types and possibly across multiple types of tumors that can fine-tune targeted therapeutics^{3,4.}

Cancer proteomic consortia have recently provided massive proteogenomic data and the initial framework for the analysis of the proteomic platforms and the integration with genomic data⁵⁻⁷. This work highlighted the rich heterogeneity of most tumor types when examined through protein, protein modifications, and in some cases metabolism and lipid composition. These studies also showed an important association between cancer driving alterations and protein levels/modifications and reconstructed oncogenic pathways relevant to prognosis and/or therapeutic decisions on the basis of proteomics changes^{8,9}. Over the past few years, it has become evident that restricting attention to individual genes and pathways is not beneficial to most cancer patients and the integrated convergence of multi-omics datasets towards druggable targets (e.g., protein kinases) driving the identity of individual tumors and/or tumor subtypes has to be charted¹⁰⁻¹². Consequently, large-scale introduction of proteomics/phosphoproteomics datasets into clinical practice remains a longed for but yet unfulfilled goal of proteogenomics resource studies.

Here, we reconstructed four functional subtypes of GBM¹³ using proteomics, phosphoproteomics, acetylomics, metabolomics and lipidomics data using the GBM dataset from the Clinical Proteomic Tumor Analysis Consortium (CPTAC). We developed a novel computational approach referred to as Substrate PHosphosite based Inference for Network of KinaseS (SPHINKS) to generate an unbiased kinome-phosphosite network and extract the Master Kinases (MKs) driving the GBM subtypes. The SPHINKS algorithm exhibited marked stability against multiple levels of perturbations of the input dataset and higher prediction power than other inference methods. We experimentally validated protein kinase C delta (PKCδ) and DNA-dependent protein kinase catalytic subunit (DNA-PKcs) as the MKs that sustain cell growth and tumor cell identity of the glycolytic/plurimetabolic (GPM) and proliferative/progenitor (PPR) functional GBM subtypes, respectively. We confirmed PKCδ and DNA-PKcs as MKs in GPM and PPR tumors from pediatric glioma (PG), breast carcinoma (BRCA) and lung squamous cell carcinoma (LSCC) cohorts classified according to the four functional classes that recapitulate the metabolic and proliferation tumor cell states. Finally, we developed a probabilistic classification tool for GBM that exhibits optimal performance in both frozen and formalin-fixed, paraffin-embedded tumor tissue for application in cancer clinical pathology.

### Proteogenomic Analysis Captures Functional Subtypes of Glioblastoma

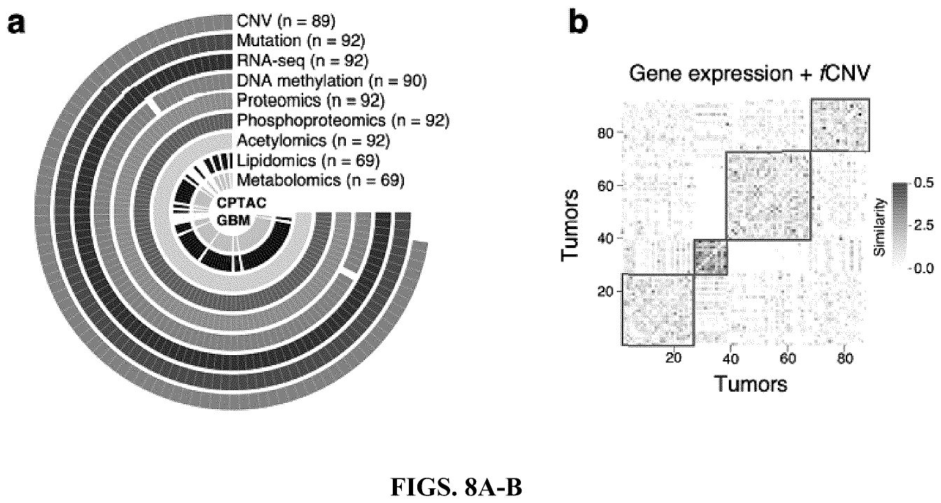

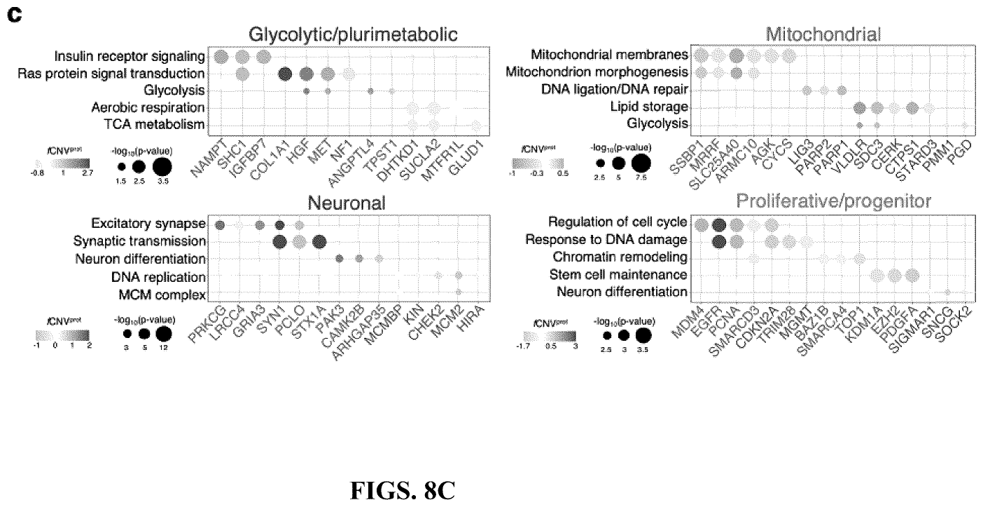

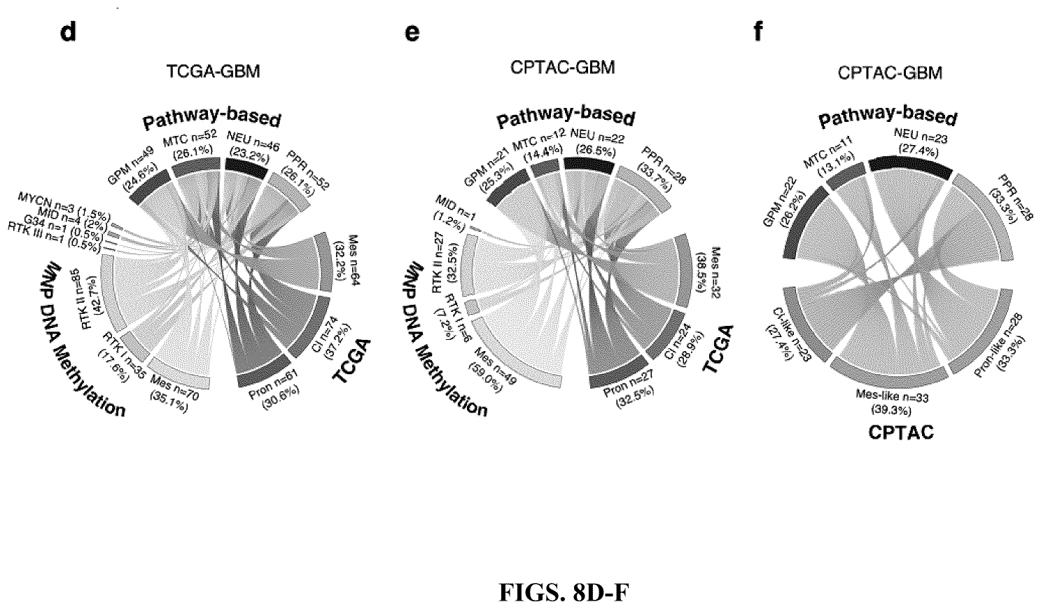

We reported a single cell-guided pathway-based classification of IDH wild type GBM that consisted of four tumor subtypes distributed along two functional axes including neurodevelopment (proliferative/progenitor or PPR and neuronal or NEU) and metabolism (glycolytic/plurimetabolic or GPM and mitochondrial or MTC)¹³. The MTC subtype was associated with better survival and sensitivity to OXPHOS inhibitors. Here, we used the proteogenomic datasets of 92 IDH wild type GBM from the Clinical Proteomic Tumor Analysis Consortium (CPTAC) cohort that was profiled by genomics, transcriptomics, proteomics, phosphoproteomics, metabolomics, acetylomics and lipidomics to explore the biology associated with the multi-omics taxonomy and uncover potential targeting opportunities for the most aggressive GBM subtypes that are still lacking rational therapeutic options (FIG. 8a)¹⁴. We implemented Similarity Network Fusion (SNF), an integrative clustering method that fuses different data types into a single network¹⁵. As functional Copy Number Variations (fCNVs)—the CNVs of genes associated with coherent transcriptomic changes in cis—and gene expression were the primary data sources in the pathway-based classifier of GBM¹³, we selected the validated fCNVs and mRNA expression as input features to build individual networks and the fused network for CPTAC samples, and obtained four stable clusters (FIG. 8b). Using 52 GBM classified according to the highest transcriptomic-based simplicity score as anchors (see Methods), we classified 33 out of 40 remaining tumors by the distance matrix of the SNF network. The differential gene expression of the four SNF clusters was enriched with the biological activities previously assigned to GPM, MTC, PPR and NEU GBM subtypes¹³.

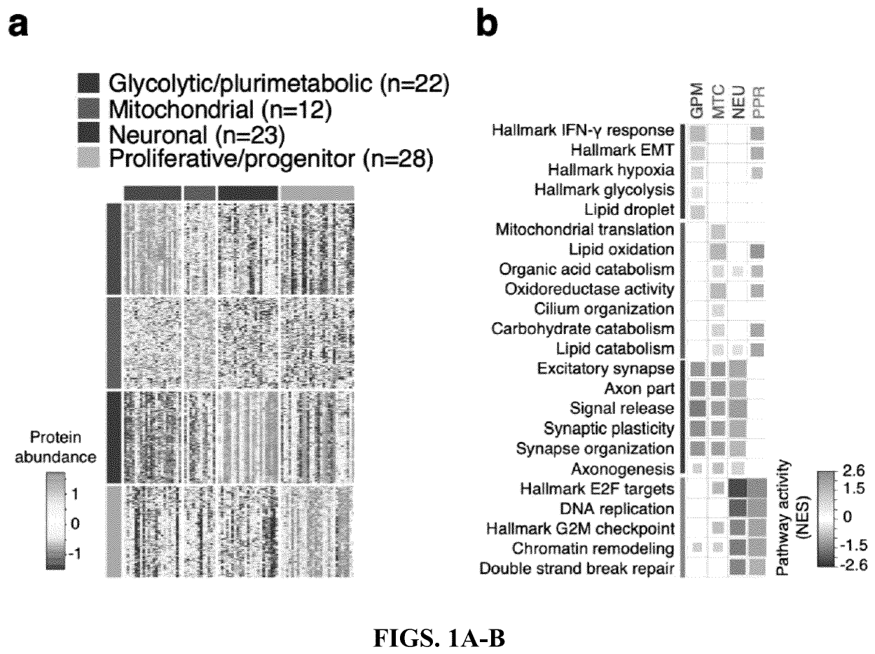

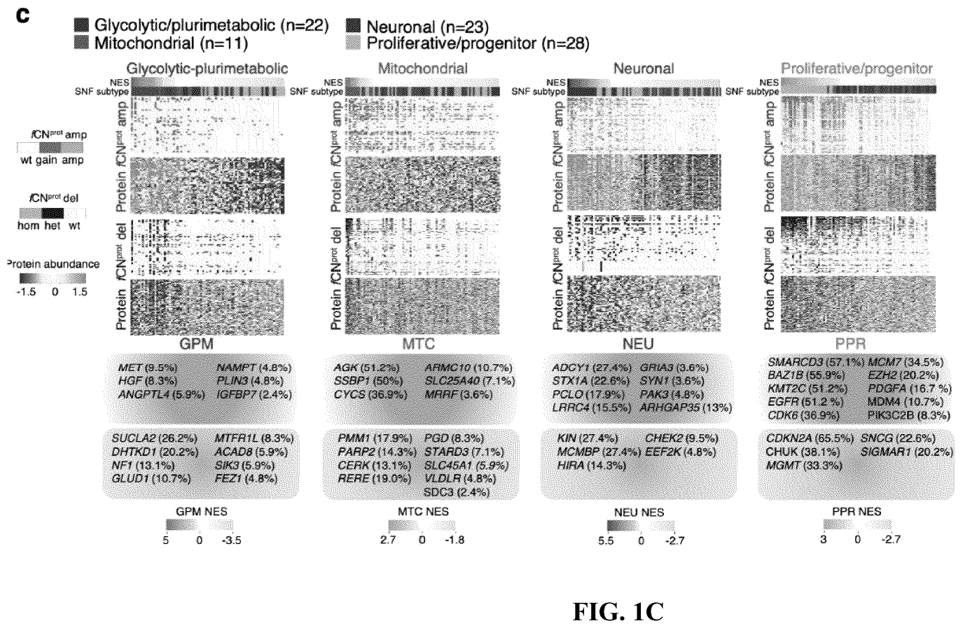

Proteogenomic studies in multiple cancers have demonstrated that DNA-level and RNA-level alterations may be insufficient to predict protein activity as transcriptomics may have limited correlation with proteomic data¹⁶⁻¹⁸. We therefore asked whether GBM subtyping obtained by the SNF approach coincided with proteomic activities biologically congruent with CNV and gene expression-guided functions. The inspection of the most differentially abundant proteins and enriched pathways revealed that the global expression of proteins faithfully recapitulated the predominant functional activities assigned to each GBM subtype by the SNF clustering (FIGS. 1a, b).

To ask whether fCNVs impact protein abundance in cis (fCNVprot), we integrated genomics, transcriptomics, and proteomics data to identify genes for which gain or loss correspondingly changed mRNA and protein expression. A total of 2,205 genes with fCNV gain and 2,837 genes with fCNV loss had concordant changes in protein abundance when compared to copy number neutral samples. Among them, 553 (25.08%) fCNVprot gains and 415 (14.63%) fCNVprot losses segregated with one of the four subtypes (FIG. 1c, see Methods). fCNV^{prot} enacted functions congruent with the established subtype biology and contributed directly to the activation/deactivation of the biological pathways marking each GBM subtype (FIG. 8c).

To provide a comprehensive comparison of our pathway-based classification with the previously proposed transcriptional and epigenetic subtypes of GBM, we selected TCGA and CPTAC datasets corresponding to 199 and 83 IDH wild type GBM respectively, with each tumor profiled by both DNA methylation arrays and RNAseq for an integrated assessment of the relationship between DNA methylation- and transcriptomic-based classifiers. We performed a three-way comparison between pathway-based, functional classifiers (four groups: GPM, MTC, PPR, and NEU), transcriptional subtypes reported by TCGA (proneural, classical, mesenchymal)¹⁹ and DNA methylation-based epigenetic subclasses described by the MolecularNeuroPathology (MNP, DFKZ/Heidelberg) group (mesenchymal, RTK I, RTK II, RTK III, MID, MYCN, G34)²⁰. The GPM subtype exhibited a clear association with the mesenchymal subtypes of the TCGA and MNP classifiers. Conversely, tumors of the MTC subtype were mapped to all TCGA and MNP subtypes, with slight preference for the RTK-II subtype in the TCGA dataset and the mesenchymal transcriptomic subtype in the CPTAC dataset (FIGS. 8d-f). Within the neurodevelopmental axis of the pathway-based classification (PPR and NEU subtypes), we found limited overlap with the TCGA and MNP classifiers, with the proneural and RTK-I subtypes contributing to most of the PPR and NEU tumors (FIGS. 8d, e). Although the epigenetic RTK-III, MID, MYCN and G34 subtypes were only minimally represented in the TCGA and CPTAC datasets, (4.5% and 1.2%, respectively), we noted that 6 of 9 or 66% of tumors were classified as PPR (FIGS. 8d, e). We also compared the pathway-based functional subtypes with the subtypes reported by the recent study from the CPTAC (proneural-like, classical-like, mesenchymal-like)¹⁴. We confirmed that the GPM subtype is mainly called Mes-like from CPTAC. However, the Mes-like CPTAC subtype also includes a significant proportion of cases of the MTC subtype (FIG. 8f), indicating that our classification uniquely discriminates tumors exhibiting alternative metabolic fluxes (GPM and MTC), divergent clinical characteristics (poor vs favorable clinical outcome) and distinct targeted therapeutic vulnerability (sensitivity to inhibitors of mitochondrial respiration by MTC but not GPM tumors)¹³. The proneural-like CPTAC subtype incorporated similar fractions of NEU and PPR, whereas the classical-like was preferentially enriched with tumors classified as PPR.

Taken together, the comparative analysis confirmed orthogonal distribution of the MTC subtype and broad mapping of PPR and NEU subtypes to proneural and RTK-I (also defined as “proneural-like”)¹⁴ subtypes of TCGA/transcriptomic and MNP/epigenetic groups, thus indicating that, with the description of PPR and NEU subtypes, the pathway-based classifier more accurately captures the neurogenesis stages than the vague definition of proneural tumor state.

### An Integrative Genetic and Clinical Outlook on Functional GBM Subtypes

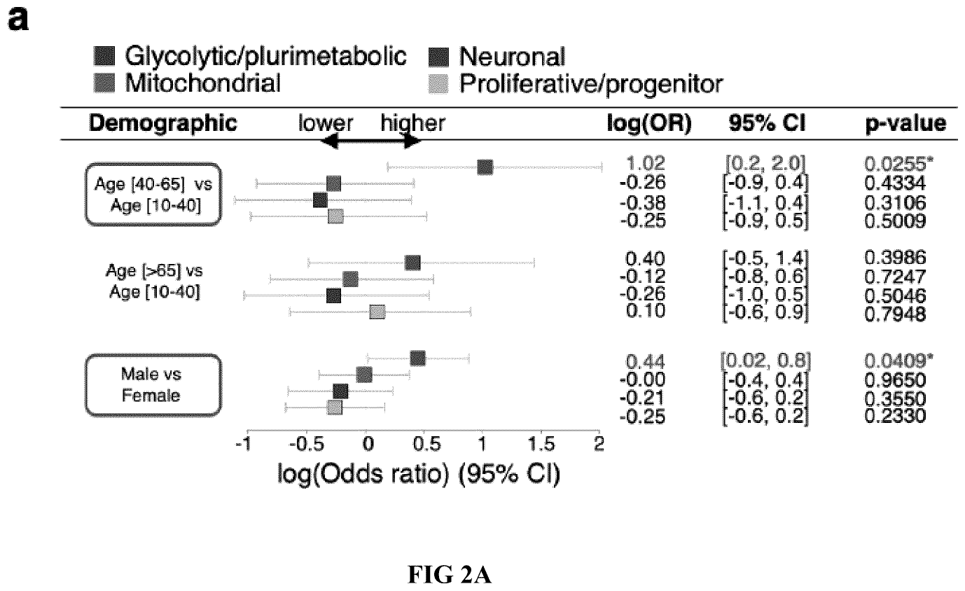

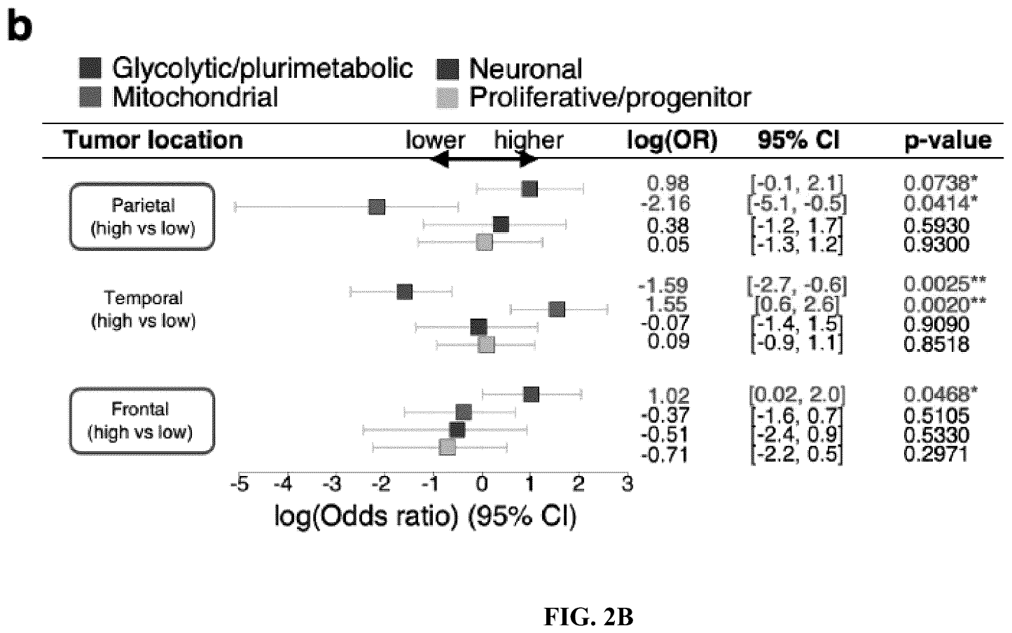

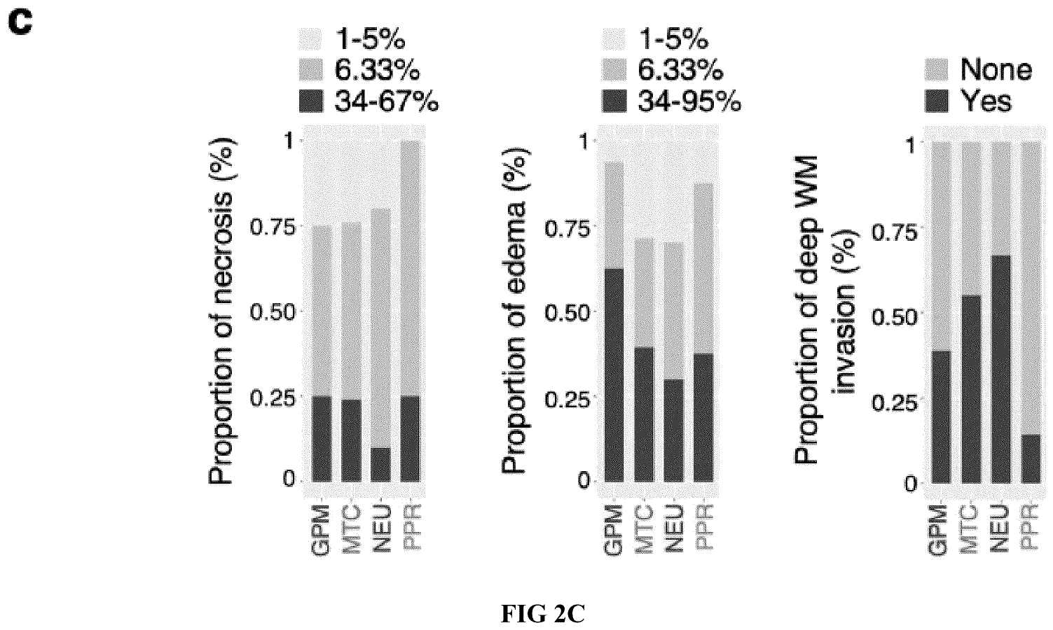

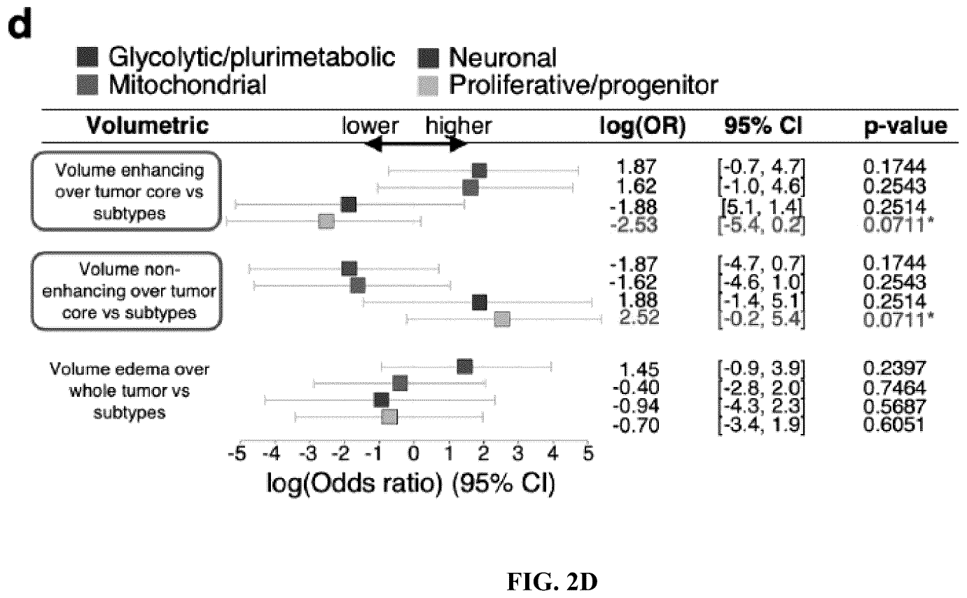

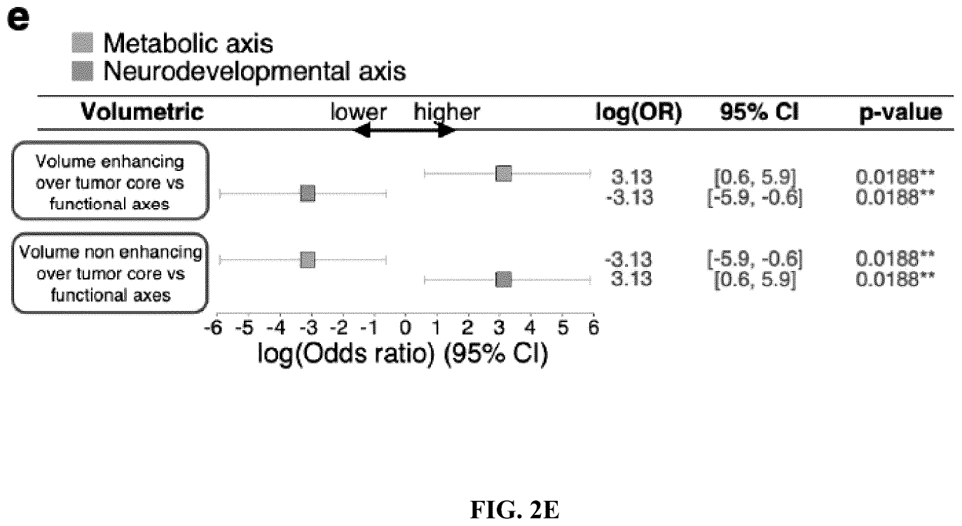

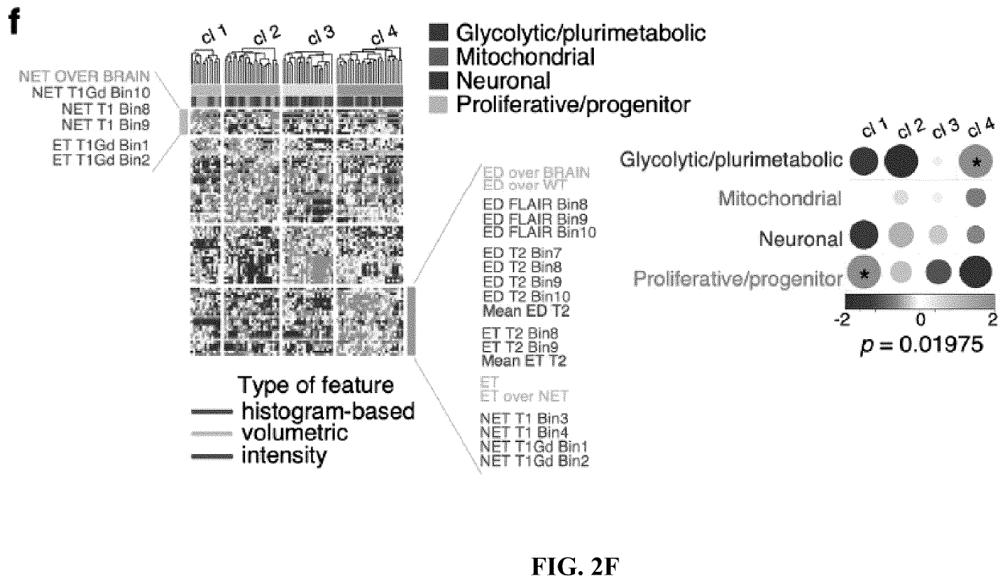

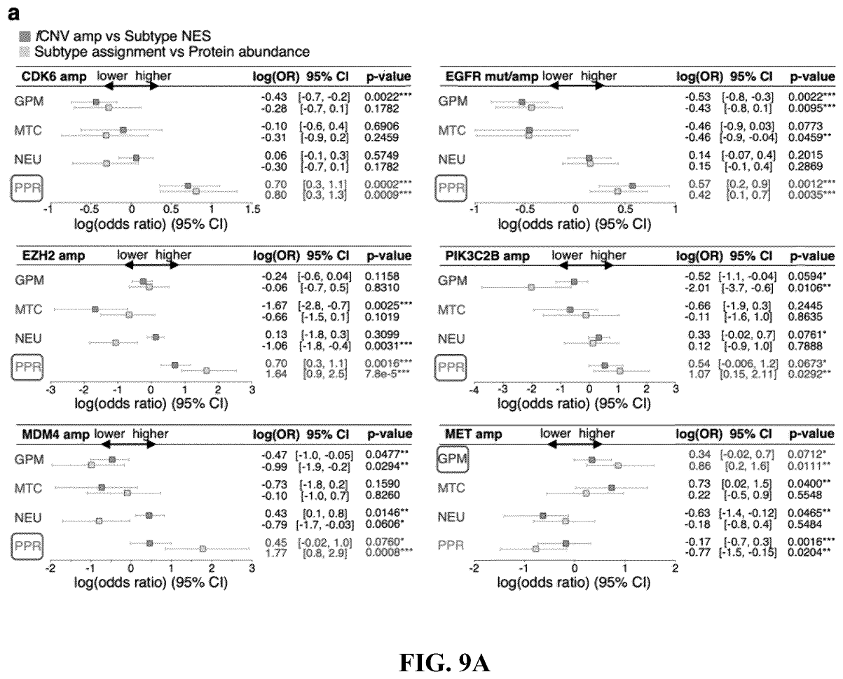

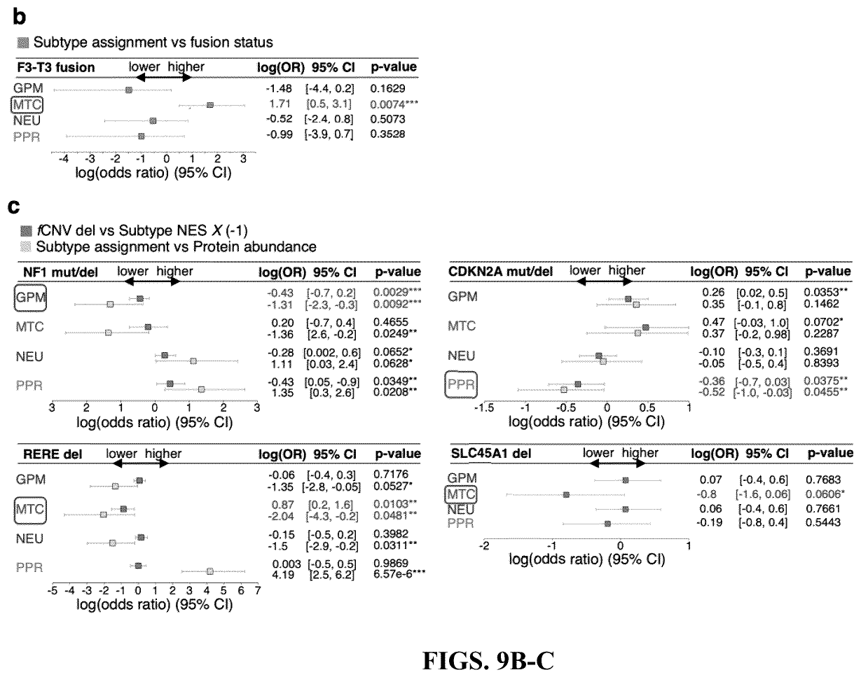

To understand whether each functional subtype of GBM reflects a unique configuration of elements that compose a distinct functional module—from genetic drivers to general clinical characteristics such as age and gender and location of the tumor in the brain or radiological features that are obtained at diagnosis by magnetic resonance (MR) imaging—we performed univariate logistic regression analyses. For the first analyses we applied a univariate logistic regression that modeled somatic pathogenic mutations and fCNV¹³ and determined odds ratios, confidence intervals and p-values for the association with each of the four subtypes. In an independent model we asked whether proteins encoded by GBM driver genes are expressed at higher or lower levels in tumors from each of the four subtype, therefore providing an orthogonal proteomic validation to the genetic associations (FIG. 2). We found that proliferative/progenitor activity predominantly associated with fCNV amplification/protein abundance of multiple GBM oncogenes (CDK6, EZH2, MDM4, amplification/mutation/protein abundance of EGFR) and fCNV deletion/mutation/protein depletion of CDKN2A, all connected to hallmarks of the PPR subtype of GBM. The glycolytic/plurimetabolic activity associated with MET fCNV amplification/mutation/protein abundance and NF1 fCNV deletion/mutation/protein depletion, which we had previously linked to the GPM subtype (FIGS. 9a, c)¹³. Confirming our previous finding of mitochondrial respiration as oncogenic mechanism of action and potential therapeutic vulnerability for FGFR3-TACC3 positive GBM, the MTC subtype was significantly associated with FGFR3-TACC3 fusion-positive tumors in the cohort of 178 GBM that we used to validate the probabilistic classifier (see below), and includes 12 FGFR3-TACC3-positive GBM (FIG. 9b)²¹. fCNV deletion of RERE and SLC45A1 genes located in the “metabolic” region of chromosome 1p36.23 previously identified as driver of the MTC subtype¹³ were associated with increased mitochondrial activity. The positive correlation between low levels of the RERE protein independently supported the association whereas the SLC45A1 protein was not present in the CPTAC proteome and therefore could not be analyzed (FIG. 9c). With the limitation of the small number of samples in the CPTAC cohort, the overall analysis indicated that protein abundance was generally a better indicator of subtype activity than CNV and somatic mutations, a finding that likely reflects the multilevel control of oncogenic protein abundance by non-genetic factors. Next, we asked whether age, gender, tumor location and radiomic imaging features exhibited specific correlations with each of the functional GBM subtypes (see Methods). Age, gender and tumor location were tested as function of subtype transcriptomic activity. Only the glycolytic/plurimetabolic activity showed a statistically significant association with male gender and the age group between 40 and 65 years (FIG. 2a). The GPM subtype was more frequently found in the frontal and parietal lobes but excluded from the temporal region. Conversely, MTC tumors were enriched in the temporal location and excluded from the parietal lobe, thus suggesting a reciprocal brain position pattern for GBM classified as GPM and MTC (FIG. 2b).

To interrogate potential associations between the functional GBM subtypes and radiomic features, we performed three different analyses based using MR imaging data available from The Cancer Imaging Archive (TCIA) portal^{22,23}. We categorized the proportion of necrosis, edema and deep white matter invasion and generated logistic regression models to correlate the proportion of tumor enhancing and non-enhancing volume of the tumor core and volume of edema of the whole tumor with subtype activity. Lastly, to capture signature features that could decode distinct GBM functional activities, we generated an unbiased clustering of histogram-based, volumetric and intensity radiomics features. The analyses showed concordantly that glycolytic/plurimetabolic activity was associated with a larger volume of edema and contrast enhancing tumor volume. Proliferative/progenitor activity was associated with higher necrosis, non-contrast enhancing volumes and lower proportion of deep white matter invasion, whereas the neuronal activity was associated with the lowest volume of necrosis and highest proportion of deep white matter invasion (FIG. 2c, d). Although the number of samples in each functional subtype was insufficient to provide statistical power, when samples within the metabolic (GPM and MTC) and neurodevelopmental axis (PPR and NEU) were combined, the analysis demonstrated that the metabolic subtypes have significantly higher contrast enhancing volume, suggesting that the blood brain barrier may be disrupted to a larger extent, while neurodevelopmental subtypes exhibited larger non-contrast enhancing volumes (FIG. 2e). The unique positive association of NEU tumors with deep white matter invasion is consistent with the concepts that this subtype has a more infiltrative behavior and neuronally differentiated GBM cells engage normal brain cells at the tumor periphery for neomorphic synaptic connections that guide invasion through white matter tracks^{13,24}. These different scenarios were also supported by the analysis of 175 radiomic features that we used to test the association of four unsupervised clusters with pathway-based GBM subtypes (χ2 test). Cluster 1 was mostly populated by PPR tumors and had high non-contrast enhancing and low contrast enhancing volumes as distinctive features. Conversely, cluster 4 was mostly enriched with GPM tumors and characterized by overrepresentation of features of edema and contrast enhancing volumes but underrepresentation of non-contrast enhancing quantitative features (FIG. 2f).

### Multi-Omics Profiling Delivers Coherent Attributes of Functional Subtypes of Glioblastoma

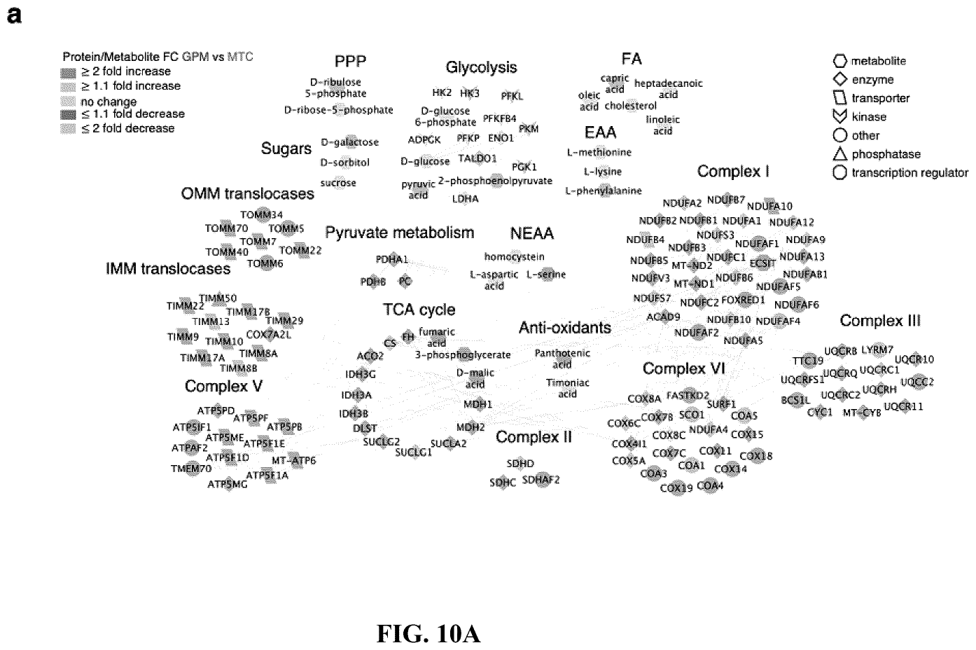

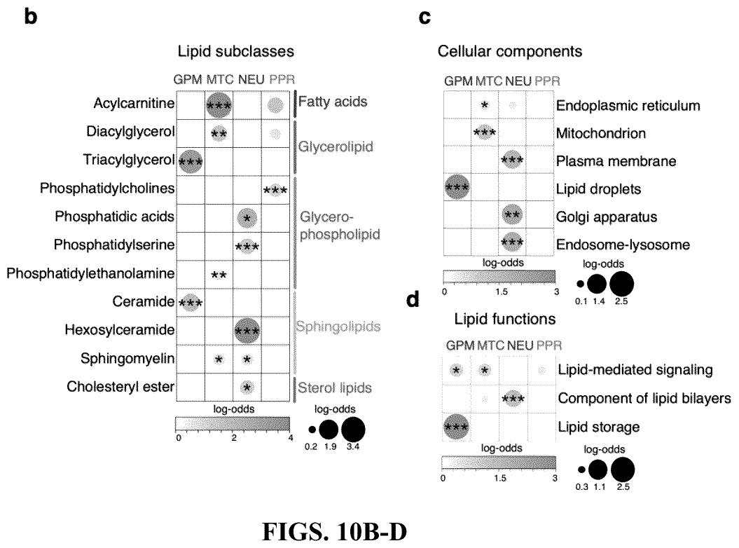

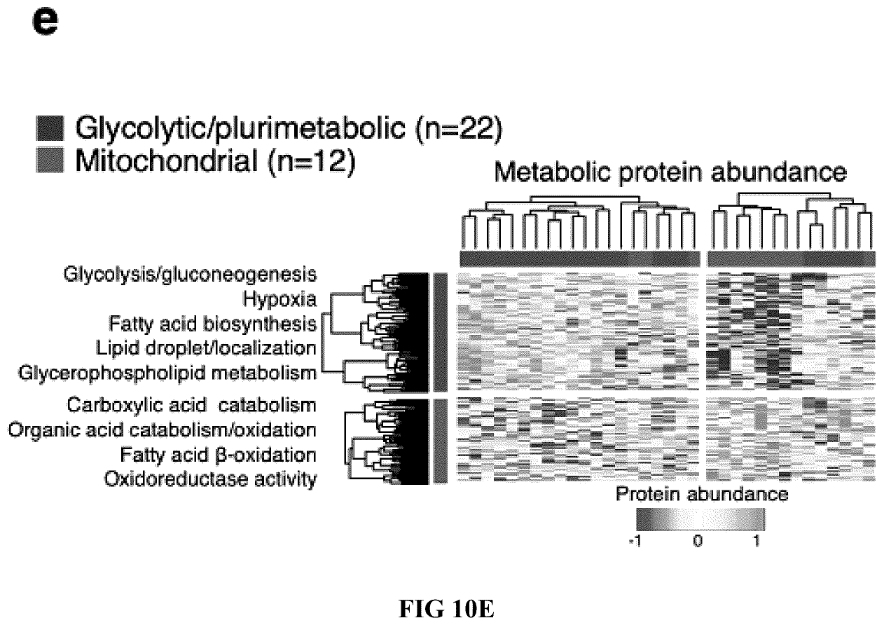

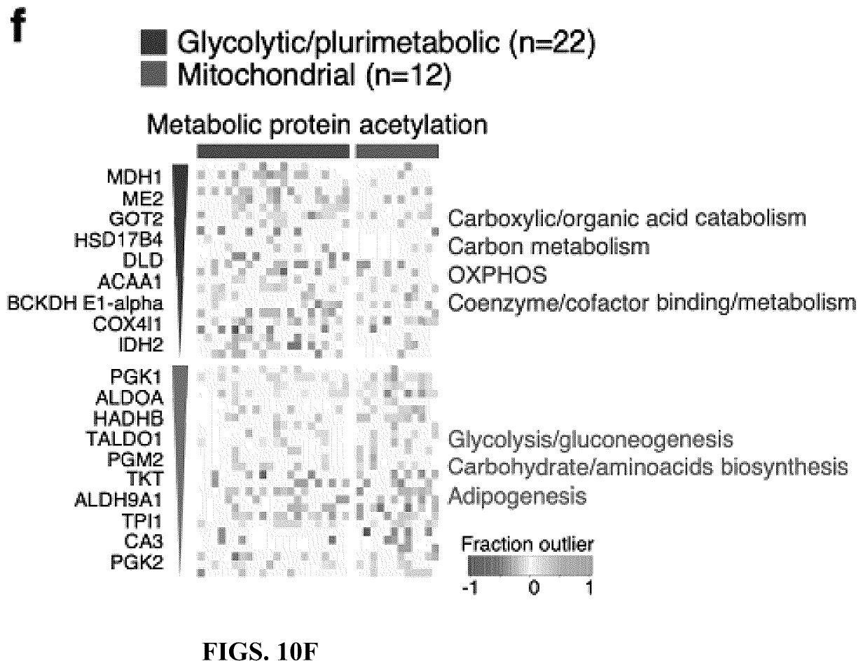

The availability of proteomics, metabolomics and lipidomics platforms in the GBM dataset prompted us to inquire whether the divergent features of GPM and MTC GBM subtypes might independently emerge from these platforms. By comparing protein profiles of GPM and MTC samples, we detected significantly higher levels of glycolytic enzymes and lower levels of mitochondrial enzymes (translocases, tricarboxyl acid [TCA]cycle and electron transport chain [ETC] enzymes) in GPM whereas the reciprocal pattern characterized the MTC subtype (FIG. 10a). Concordantly, GPM GBM was preferentially enriched with metabolic intermediates of glycolysis, the pentose phosphate shunt, fatty acids, sugars and essential amino acids whereas MTC GBM contained higher levels of TCA cycle intermediates (e.g. malic and fumaric acid), antioxidants and non-essential amino acids (FIG. 10a).

The analysis of lipidomic data using the LION (Lipid Ontology) enrichment tool²⁵ showed that the GPM subtype had the highest abundance of triacylglycerol, functionally involved in lipid storage, and ceramide, which has been reported to cause mitochondrial dysfunction (FIGS. 10b-d)²⁶⁻²⁸. Conversely, MTC GBM accumulated high levels of acyl carnitine, an integral component of the mitochondrial fatty acid oxidation pathway²⁶, and diacylglycerol, a lipid second messenger required for membrane fusion and fission²⁹. The different lipid composition of GPM and MTC GBM is highlighted in the aggregated analysis of lipid cellular components and functions with GPM enriched for constituents of lipid droplets and MTC GBM enriched for lipids involved in mitochondrial biogenesis³⁰⁻³¹ (FIGS. 10c, d, “Cellular components”, “Lipid functions”). Within the neurodevelopmental axis of GBM, PPR contained elevated phosphatidylcholines, which are required for cell cycle progression³² whereas NEU tumors were enriched in sphingomyelin, phosphatidylserine, hexosyl-ceramide, and cholesteryl ester, all of which are essential components of the myelin sheath that surrounds nerve cell axons^{33,34}, and phosphatidic acid, a central intermediate for the synthesis of neuronal membrane lipids (FIGS. 10b-d)³⁵.

The importance of lysine acetylation in the regulation of chromatin dynamics and gene expression of nuclear proteins has long been appreciated³⁶. Recently, lysine acetylation has emerged as a post-translational modification for the regulation of cytoplasmic proteins with crucial metabolic activities, and deregulated acetylation of metabolic enzymes has been proposed to drive the metabolic reprogramming of cancer cells³⁷. Thus, we inquired whether lysine acetylation might also regulate the metabolic activities associated with GPM and MTC GBM subtypes, possibly revealing potential targets for intervention. To establish a baseline signature of the metabolic proteome, we performed unsupervised clustering of metabolism-related proteins differentially expressed between MTC and GPM tumors and obtained two clusters, the first enriched with GPM tumors and characterized by the accumulation of proteins involved in glucose, amino acid and lipid metabolism, and the second enriched with MTC samples and characterized by accumulation of proteins associated with mitochondrial metabolism (FIG. 10e). Next, to identify subgroup-specific protein acetylation events we applied the outlier enrichment analysis (Black Sheep)³⁸ to acetylated proteins, thus selecting uncommonly hypoacetylated or hyperacetylated proteins. In contrast to the global proteomic results, we found that the highest acetylated metabolic proteome in GPM samples includes mitochondrial enzymes (malate dehydrogenase 1, MDH1; malic enzyme 2, ME2; aspartate aminotransferase, GOT2; isocitrate dehydrogenase 2, IDH2, acetyl-CoA acyltransferase 1, ACAA1) (FIG. 10f) whereas MTC samples exhibited high levels of acetylation of enzymes implicated in glycolysis and the pentose phosphate pathway as well as in amino acid biosynthesis and adipogenesis (ALDOA, PGK1, PGK2, PGM2, TALDO1, TKT) (FIG. 10f). As acetylation has typically been viewed as an inhibitory post-translational modification for the activity of metabolic enzymes³⁹, these results indicate an additional level of coordination of the alternative metabolic reprogramming in the metabolic subtypes of GBM.

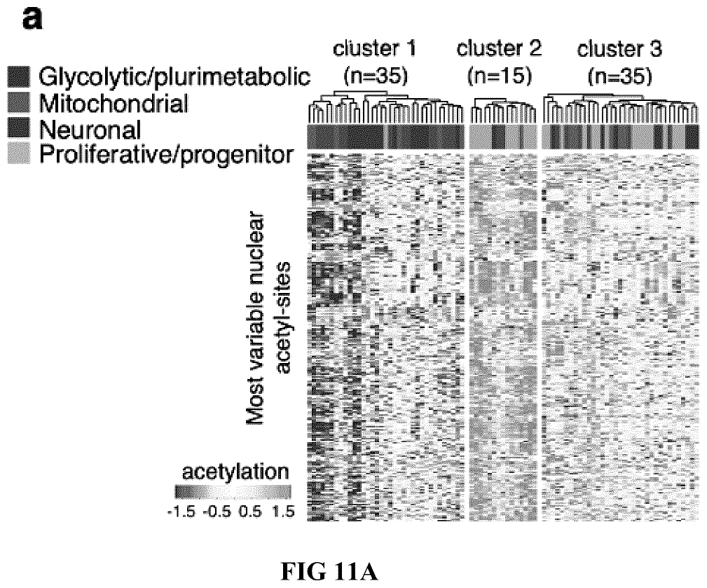

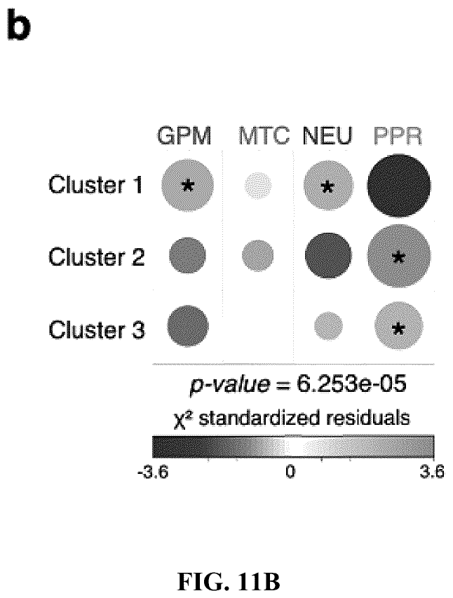

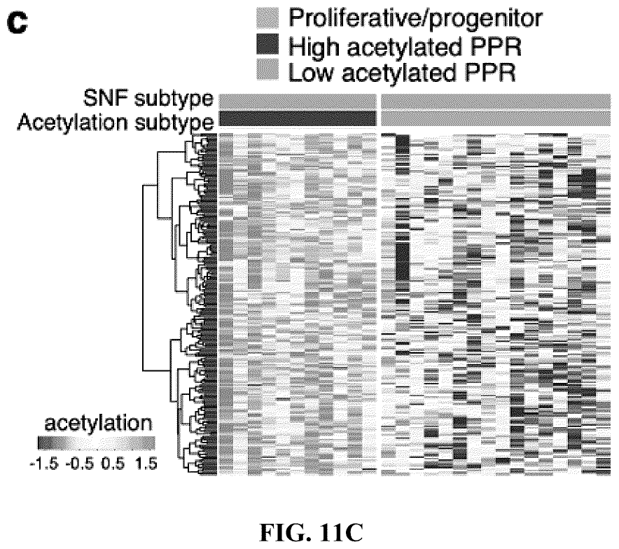

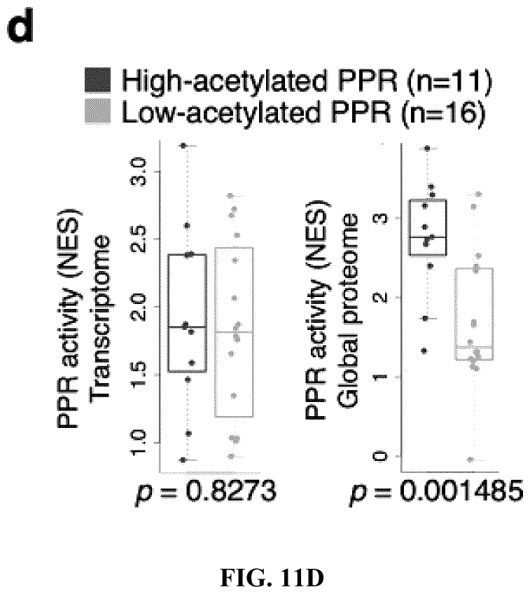

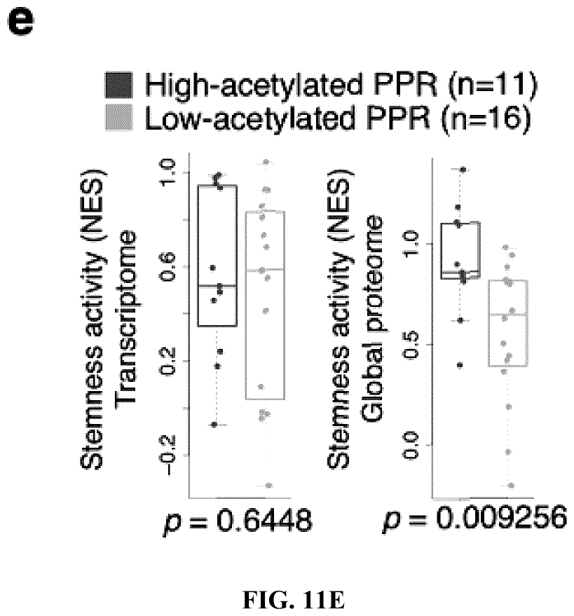

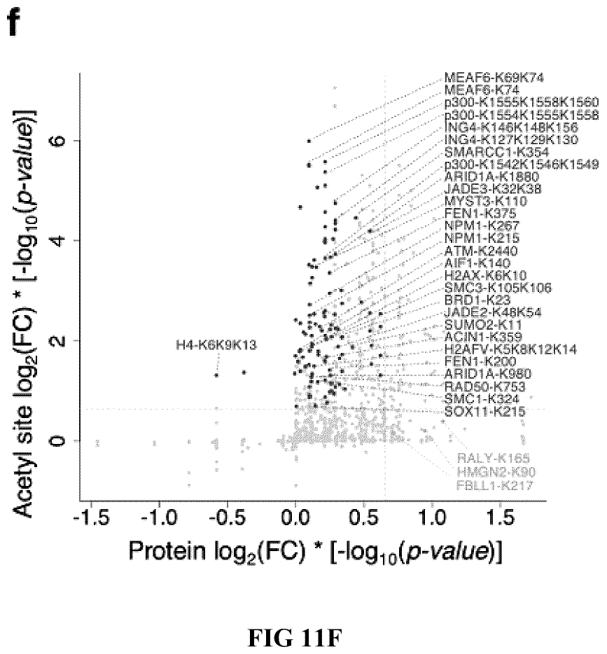

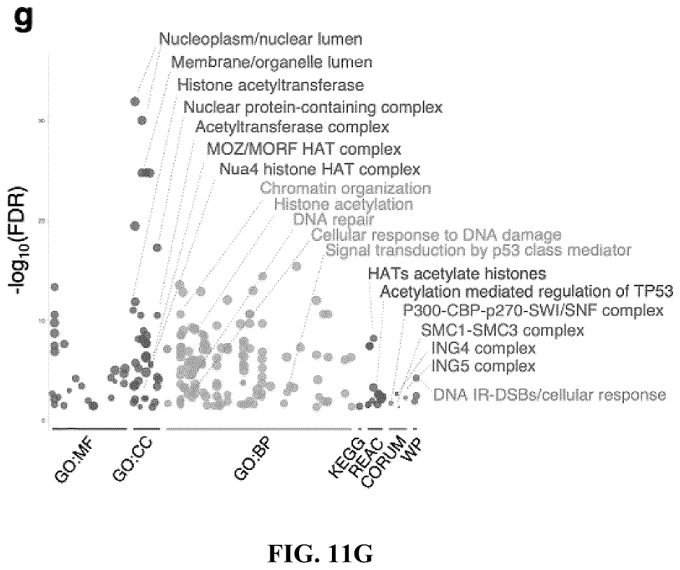

We then examined the pattern of nuclear protein acetylation across GBM subtypes. Unsupervised clustering of the most variable nuclear protein acetylation sites uncovered three distinct clusters (FIG. 11a). Cluster 1 was acetylation cold and was enriched in GPM and NEU tumors. Cluster 2 included tumors with the highest acetylation and was almost exclusively composed by PPR samples. Cluster 3 was an intermediate/low acetylation cluster that included 46% of PPR samples (16 tumors) intermixed with GPM, NEU and MTC tumors (FIG. 11b). Thus, based on nuclear protein acetylation, the PPR GBM subtype appears to be divided into two subgroups, exhibiting high and low nuclear protein acetylation, respectively (FIG. 11c). The tumors in the high-acetylation PPR sub-cluster also exhibited the highest proliferation/stemness scores inferred from proteomics but not transcriptomics, thus highlighting the specific role of the post-translation acetyl modification in the PPR subtype (FIGS. 11d, e). Differential acetylation of PPR GBM among high acetylation and low-acetylation sub-clusters involved specific acetylation sites in the absence of changes in the corresponding protein levels and targeted histone and non-histone acetyltransferases (lysine acetyltransferases, KATs) whose enzymatic activity is known to be activated by auto-acetylation⁴⁰⁻⁴⁶. Such activation is clearly manifested in high-acetylation PPR by the elevated level of acetylation of lysines in the HAT domain of the acetyltransferase p300 (K1554, K1555, K1558, K1560) and other functionally similar residues in the HAT domain of other KATs such as members of the MYST complexes (MEAF6, ING4, JADE2, JADE3, and MYST3; FIG. 11f). The latter are known to introduce acetylated marks upon histones H2, H3 and H4, which were recovered as hyperacetylated modifications of H2AX, H2AFV, and HIST2H4B in high-acetylation PPR. Besides KATs and histones, hyperacetylated proteins in high-acetylation PPR were enriched for chromatin modifying enzymes and enzymes involved in DNA Damage Repair (DDR) and DNA replication stress (RS) (ATM, RAD50, NPM1, FEN1, SMC1, SMC3) suggesting that acetylation contributes to the activation of these biological functions in the PPR subtype (FIG. 11g)⁴⁷.

### Sustained Replication Stress and DNA Damage/Repair Signaling Characterizes PPR GBM

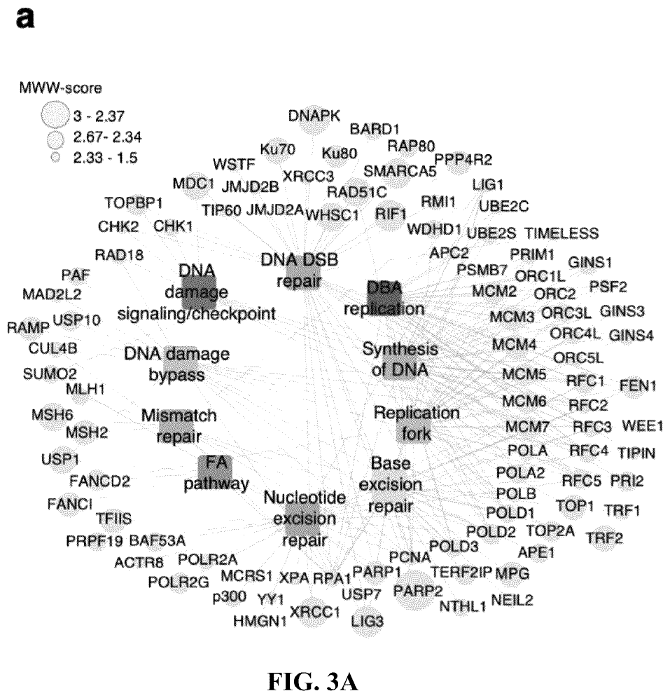

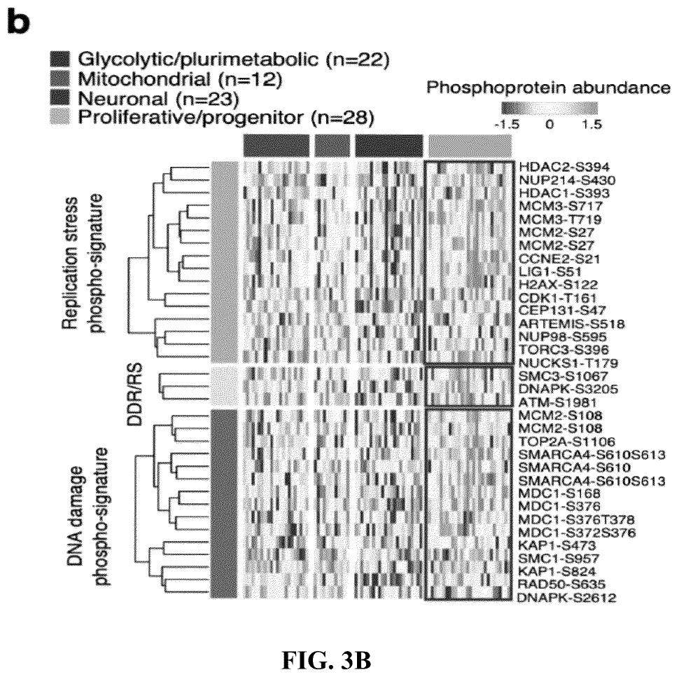

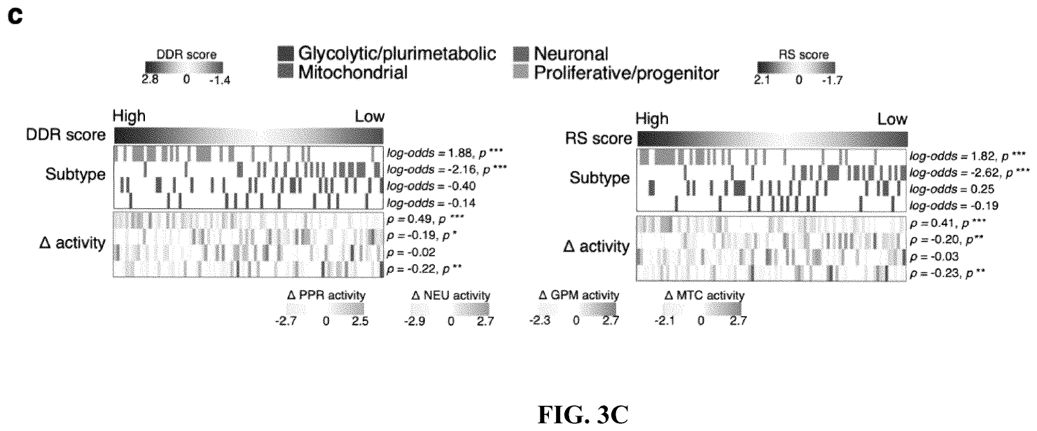

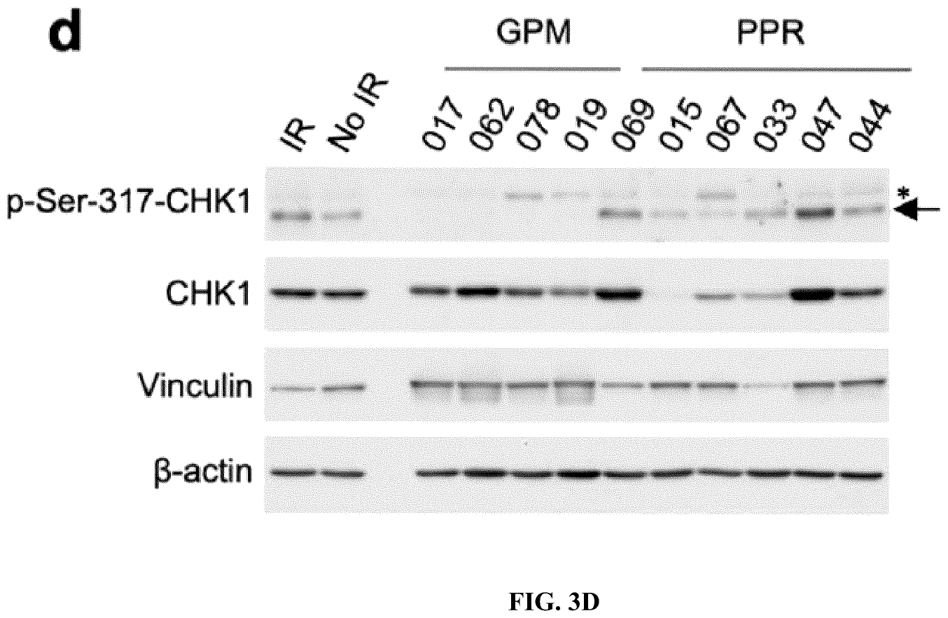

The proteomic profiling of PPR GBM unambiguously combined molecular marks of proliferation with activation of the DDR (FIG. 1b). Moreover, a proteomic analysis focused on proteins and pathways implicated in the DDR and specifically activated in PPR tumors showed the over representation of DNA replication/replication fork and double-strand break repair, suggesting that enhanced DNA replication stress (RS) may promote DNA damage/repair signaling in PPR cells and represent a point of intervention in this GBM subtype (FIG. 3a). To test this hypothesis, we performed data mining and ontology integration of multiple studies reporting mass-spectrometry based protein phosphorylation sites enriched in cells treated with irradiation, which causes DNA double-strand break (DDSB) lesions, ATR inhibitors or hydroxyurea that induce RS⁴⁸⁻⁵². We generated a list of 15 and 16 experimentally validated phospho-sites consistently elevated in and thus specific for cells undergoing DDSB and RS, respectively and 3 phosphorylation sites common to DDSB and RS. The levels of 11 (73.3%) and 10 (62.5%) of DDR and RS signature phosphosites, respectively, were increased in PPR tumors compared to all other tumors, a result that is consistent with the heightened pressure to activate DDR signaling in PPR cells (FIG. 3b). We used DDR and RS phospho-proteomic signatures to compute DDR and RS phosphoprotein enrichment scores for each GBM tumor. The analysis returned higher DDR and RS scores in the PPR subtype than other subtypes, with the NEU group characterized by the lowest scores (FIG. 3c, upper panels). To further establish the strength of the association between DDR/RS levels and PPR GBM, and the significance of proteomic-based subgroups for this association, we classified tumors according to a score calculated as the difference between proteomic and transcriptomic subtype activity. Again, a higher score was found in PPR tumors (FIG. 3c, bottom panels). Western blot analysis using CHK1 Ser-317 phosphorylation as basal DDR biomarker of ATR-activated CHK1⁵³ showed that GBM-Patients-Derived Organoids (PDOs) classified within the PPR subtype¹³ exhibited higher levels of basal RS/DNA damage than GPM PDOs (FIG. 3d). Together, these results provide orthogonal cross-validations of the phosphoproteomic finding and converge on the elevation of DDR and RS activities as unique hallmarks of the PPR subtype of GBM.

### An Unbiased Master Kinase Analysis Uncovers GBM Subtype-Specific Protein Kinases and Actionable Dependencies

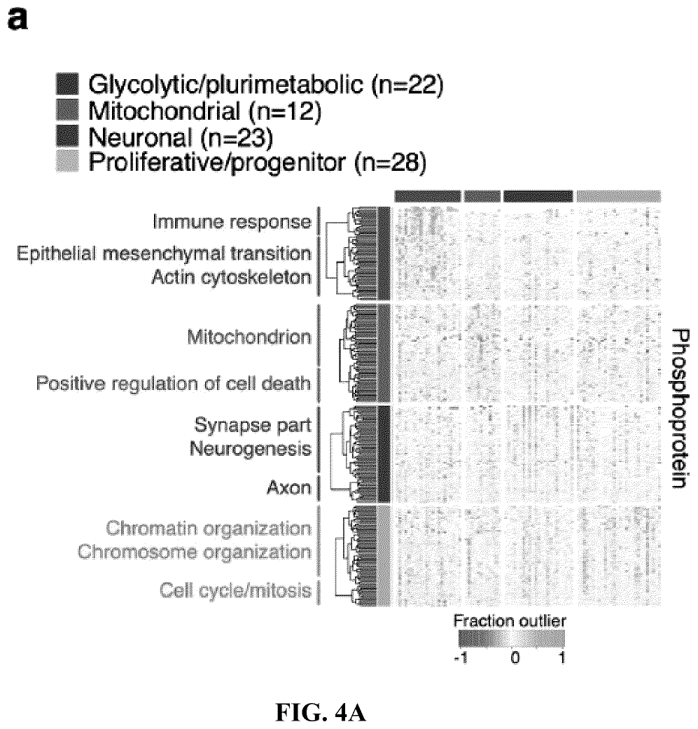

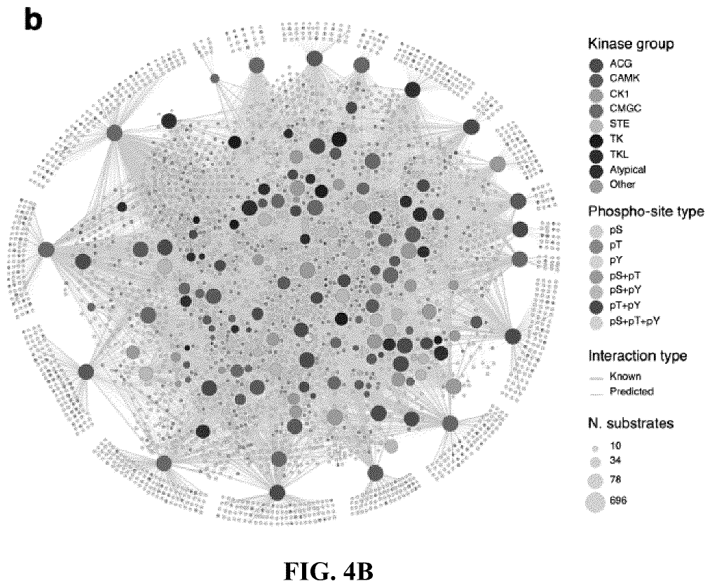

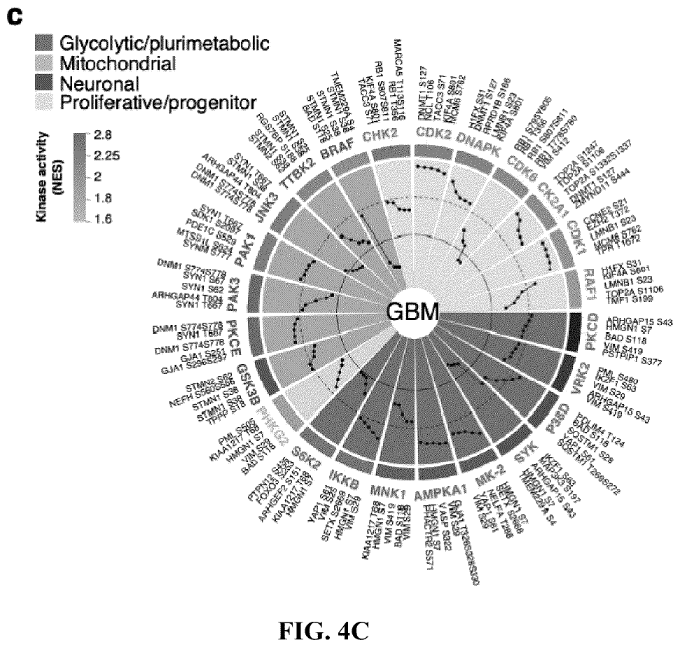

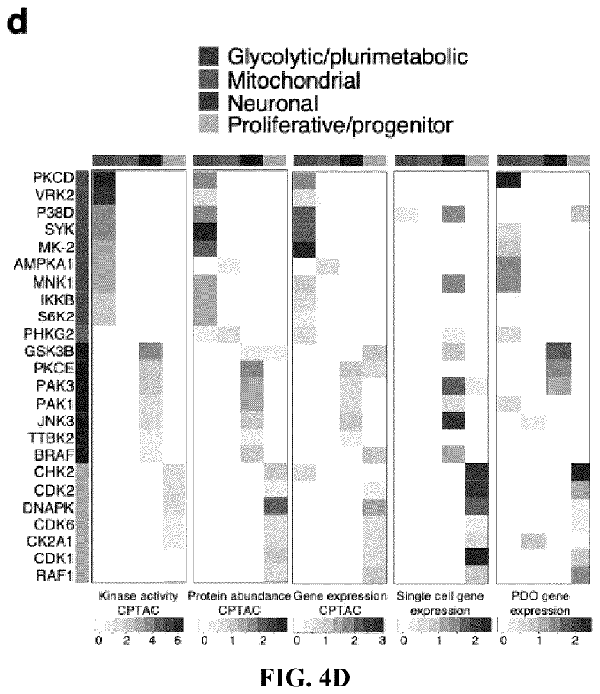

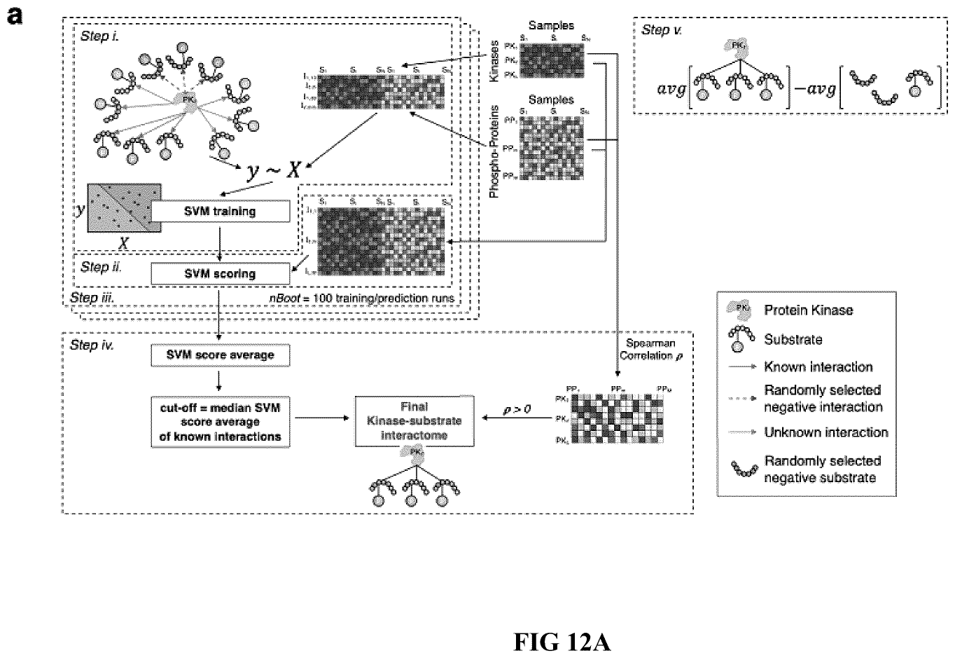

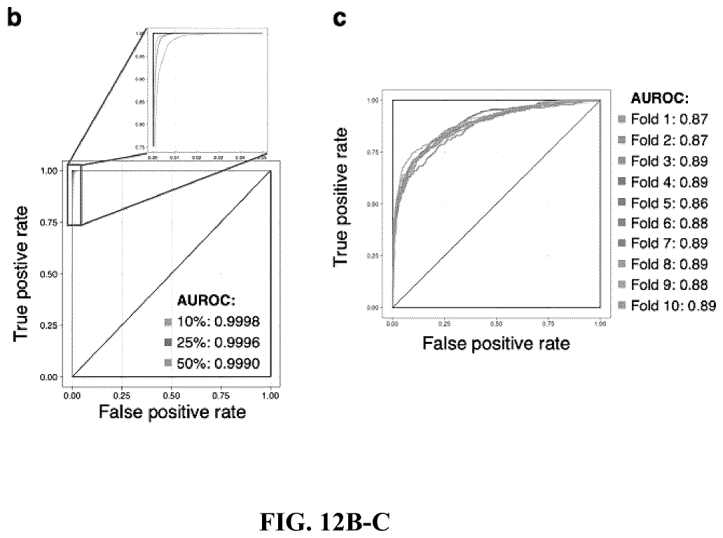

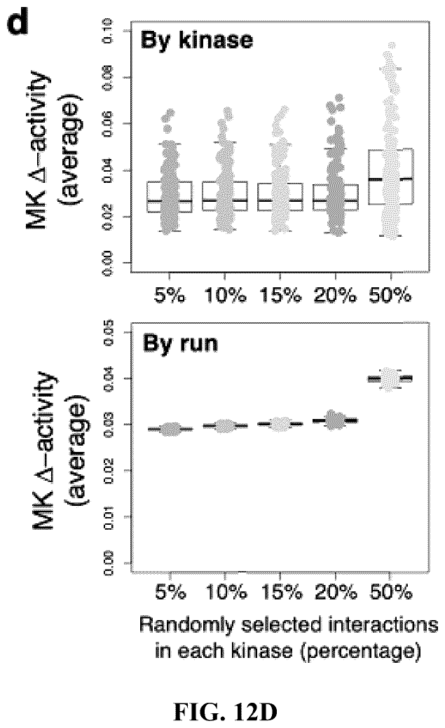

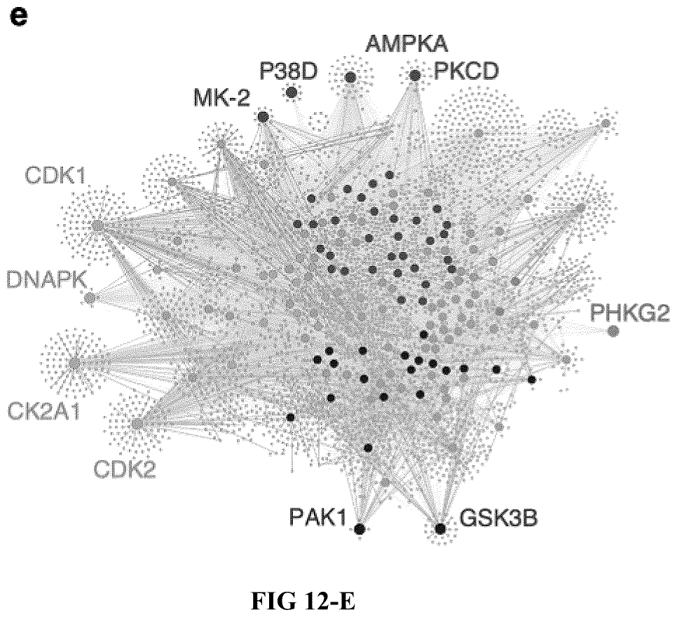

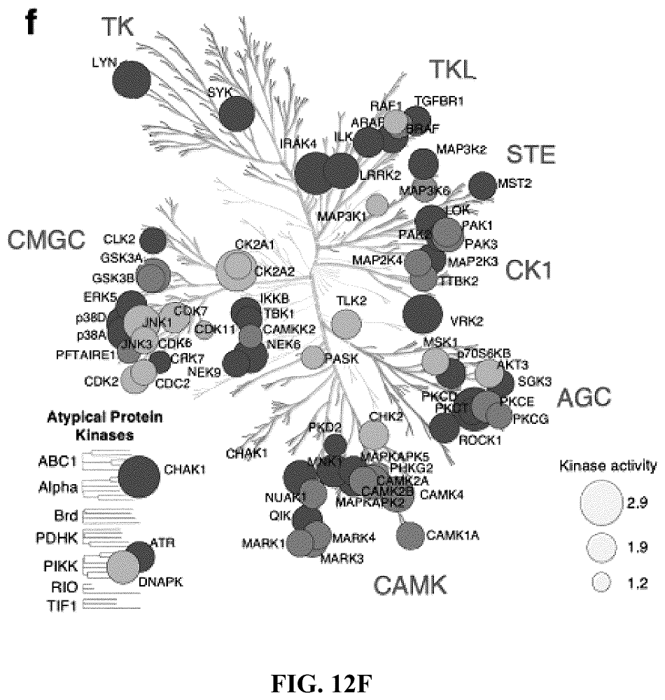

Phosphorylation by protein kinases regulates structure, function and interaction partners of proteins and plays important roles in cellular signaling of normal and disease conditions. In cancer, mass spectrometry-based phospho-proteomics creates an optimal scenario to discover biology that may not be captured from the analysis of transcriptomics and proteomics data, and trace the activity of targetable protein kinases⁵⁴. To begin exploring the phosphoproteomics landscape of GBM subtypes and their organization, we catalogued phosphosites specific for each GBM subtype. Next, we applied the outlier enrichment analysis BlackSheep algorithm and obtained four phosphosite modules of overrepresented pathways that summarized the biological hallmarks previously assigned to individual subtypes (FIG. 4a). Having acquired the knowledge of the GBM phosphorylome, we sought to link these phosphosites enrichment to the activity of GBM subtype-specific protein kinases, the most appealing targets in cancer. To unravel the protein kinases that are activated in the context of a GBM-specific network and are responsible for the identity of each subtype, we developed Substrate PHosphosite based Inference for Network of KinaseS (SPHINKS). SPHINKS integrates proteomics and phospho-proteomics profiles to first build a network of kinase-phospho-substrate pairs that are scored according to the strength of their interaction across all samples and obtain a GBM kinase-substrate interactome (FIG. 4b). The GBM-specific kinase-phosphosite interaction network was generated using a semi-supervised support vector machine (SVM) algorithm trained on labeled data of experimentally validated kinase/substrate phosphosite pairs from the PhosphoSitePlus database. When applied to proteome and phosphoproteome of GBM, SPHINKS produced a kinase-phosphosite interactome comprising 13,866 interactions between 154 kinases and 3,186 phospho-substrates (FIG. 12a, steps i-iv). We benchmarked SPHINKS at multiple levels. To assess the impact of missing data in the kinase-phosphosite interactome, we performed a comparative analysis of controlled simulations that reconstruct kinase-phospho-substrate interaction network from the un-imputed phospho-proteomics matrix of the CPTAC-GBM phosphosites lacking missing values (gold standard, n=7,302 phosphosites). Additional matrices were then generated using different ratios of phosphosite missing values (r=10%, 25%, 50%) by randomly removing values from the unimputed matrix and imputing them using DreamAI⁵⁵. SPHINKS was applied to these matrices and results compared. The ROC analysis in FIG. 12b shows that, regardless of the thresholds of missing values selected for the analysis, the AUC is consistently close to 1, indicating that, at least within the ratio of 50% corresponding to the cut-off selected in the pre-processing of the proteomics/phospho-proteomics data, the output of SPHINKS is not affected by missing values. Next, we evaluated the performance of SPHINKS to predict the correct phosphor substrates as kinase targets. We generated ROC curves of a 10-fold cross-validation by randomly dividing the experimentally validated kinase-phospho-substrate interactions from PhosphoSitePlus into 10 subsets for training (9 of 10) and testing (1 of 10). Training sets were composed of experimentally validated kinase-phospho-substrate interactions (positive training set) and randomly selected unknown interactions (negative training set). Test sets were independent selections of validated kinase-phospho-substrate interactions and unknown interactions. SVM output average scores for each interaction in the test sets were used to build ROC curves, and AUC values of all iterations between 0.86-0.89 indicates high accuracy of prediction (FIG. 12c). Since some of the selected unknown phospho-sites that we used as negative test set might be real substrates of a particular kinase, the AUC values are likely underestimated. To determine the stability of kinase activity estimates inferred by SPHINKS, we generated 100 independent networks for each kinase and perturbed them by replacing a pre-determined percentage of phospho-substrates corresponding to p=bottom 5%, 10%, 15%, 20%, 50% of the SPHINKS score with random phosphosites. We then determined the impact of the kinase-phospho-substrate interaction mis-classifications on kinase activity estimates by SPHINKS. For each kinase and network (run), we independently generated average Δ-activity scores (percent) as the difference of activity between unperturbed and perturbed networks. The results indicated a remarkable stability of the kinase activity estimate inferred by SPHINKS even for perturbations of 20% of the kinase-phospho-substrate interactions (median Δ-activity=3% in both analyses). The first detectable increase of Δ-activity only emerged for perturbations of 50%, with a median of 4% in the analyses of kinase and run and a maximum of 10% in the kinase analysis (FIG. 12d). Taken together, these results indicate that the stable kinase activity estimation by SPHINKS is primarily sustained by the high-score kinase-phospho-substrate interactions with minimal contribution from mis-classified, low-ranking interactions.

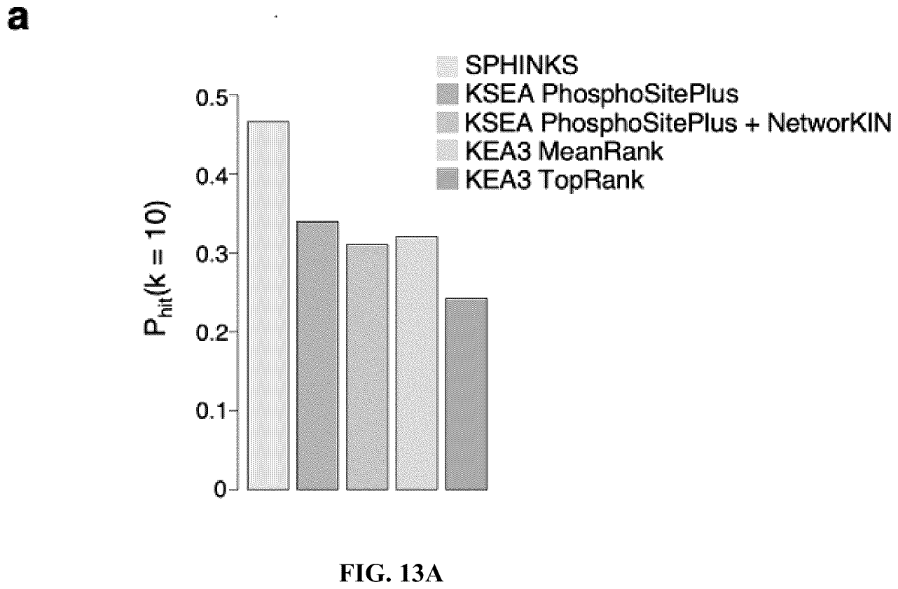

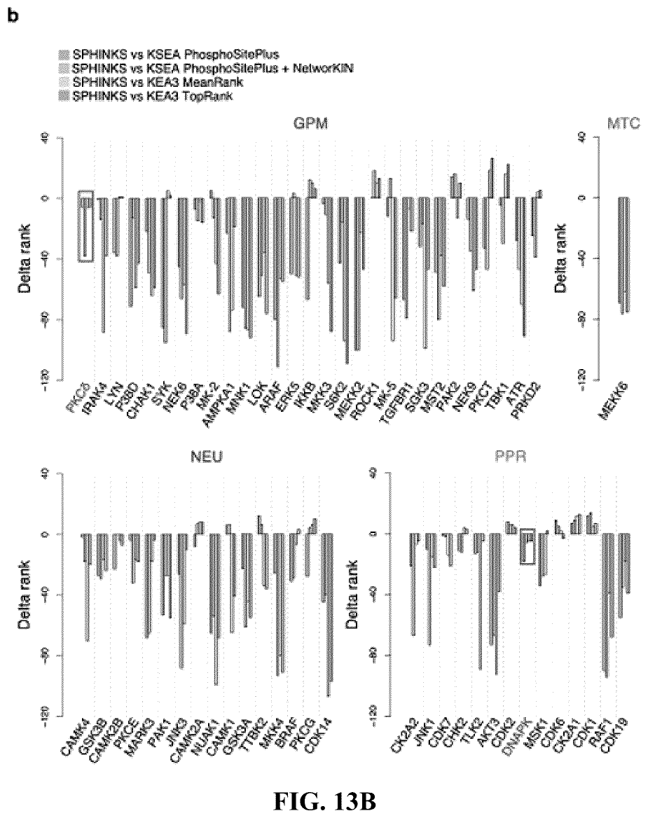

Having benchmarked SPHINKS, we sought to uncover Master Kinases (MKs) associated with distinct GBM subtypes. We designed a single sample MK analysis in which we computed the weighted strengths of connectivity between kinase and predicted substrate phosphosites against a set of randomly selected phosphosites for each tumor (FIG. 12a, step v) and used the robust Mann-Whitney-Wilcoxon (MWW) test previously validated for the identification of tumor subtype-specific features⁵⁶ to weigh each MK contribution in a tumor subtype ranking step [log₂(FC)>0.3, p<0.01; FIG. 4c]. GPM, PPR and NEU GBM exhibited rich and interconnected kinase-substrate networks as opposed to the MTC subtype that was sustained by a more limited network organized on few kinases (FIG. 12e). Mapping the predicted subtype-specific MKs onto the human kinome tree showed a random distribution of the active subtype-specific kinases across the different kinase families, thus indicating that the output of the SPHINKS-MK analysis cannot be predicted by the simple associations between distinct kinase families and individual GBM subtypes (FIG. 12f). To prioritize the identification of MKs with specific tumor cell-intrinsic activities in each GBM subtype, we orthogonally validated each subtype-specific MK across multiple platforms, including the abundance of the kinase protein and mRNA in bulk GBM and the expression of the kinase mRNA in scRNAseq data from 17,367 individual cells and 79 GBM PDOs tumor cells¹³. These analyses showed that the mRNA and protein levels of most of the kinases identified by SPHINKS-MK were also specifically upregulated in the tumor cells of the respective subtype (FIG. 4d). As additional tests, we benchmarked SPHINKS-MK against the previously published kinase-substrate inference methods Kinase-Substrate Enrichment Analysis (KSEA)⁵⁷-5⁸ and Kinase Enrichment Analysis 3 (KEA3)⁵⁹. Unlike SPHINKS that reconstructs context-specific kinase-phospho-substrate networks and detects potentially novel kinase-substrate interactions, KSEA and KEA3 derive the activity of each kinase from pre-known networks that include experimentally validated phospho-substrates. For KSEA, we derived kinase activities using experimentally validated kinase-phospho-substrate interactions from PhosphoSitePlus (KSEA PhosphoSitePlus) or including also predicted relationship from NetworKIN (KSEA PhosphoSitePlus+NetworKIN). For KEA3, we also considered two approaches, MeanRank and TopRank for ranking the integration of kinase activity that was obtained from 11 protein-protein and kinase-substrates interaction libraries. Against this background of existing methods, we benchmarked SPHINKS using two independent approaches. First, we used a dataset reporting the downstream changes in the abundance of phospho-proteins after perturbation by stimulators or inhibitors of upstream kinases58,60,61. The benchmark dataset was generated by assembling 24 studies that together encompassed 103 kinase-perturbation annotations for 30 different kinases and 61,181 phospho-sites identified in at least one perturbation (the “gold standard”). As canonical benchmarking methods like AUROC and precision at recall 0.5 consider high number of predictions, unlikely to be experimentally validated, we utilized the metric defined as “top-k-hit” (see Methods), which focuses on the top k kinase predictions61. This analysis demonstrated that SPHINKS had superior ability to correctly identify the perturbed kinases with highest activity scores than other methods (SPHINKS: 47%; KSEA PhosphoSitePlus: 34%; KSEA PhosphoSitePlus+NetworKIN: 31%; KEA3 MeanRank: 32%; KEA3 TopRank: 24%, FIG. 13a).

Finally, we ranked activity of 129 kinases common to all 5 methods for each GBM functional subtype and calculated the Δ-rank score as the difference between the rank of the kinase inferred by SPHINKS and each of the other methods on CPTAC-GBM proteomic/phosphoproteomic data. A Δ-rank score lower than 0 on an experimentally validated kinase indicates that SPHINKS returns a higher activity and hence performs better than the other approaches. A Δ-rank score higher than 0 indicates the opposite result. In all comparisons, most of the kinases exhibited a negative Δ-rank score (87%, 78%, 77% and 73% versus KSEA PhosphoSitePlus, KSEA PhosphoSitePlus+NetworKIN, KEA3 MeanRank and KEA3 TopRank, respectively), indicating that SPHINKS has a consistently higher predictive power than the other approaches (FIG. 13b).

### Identification of PKCδ and DNA-PKcs as Crucial Subtype-Specific and Therapeutically Actionable MKs in GPM and PPR

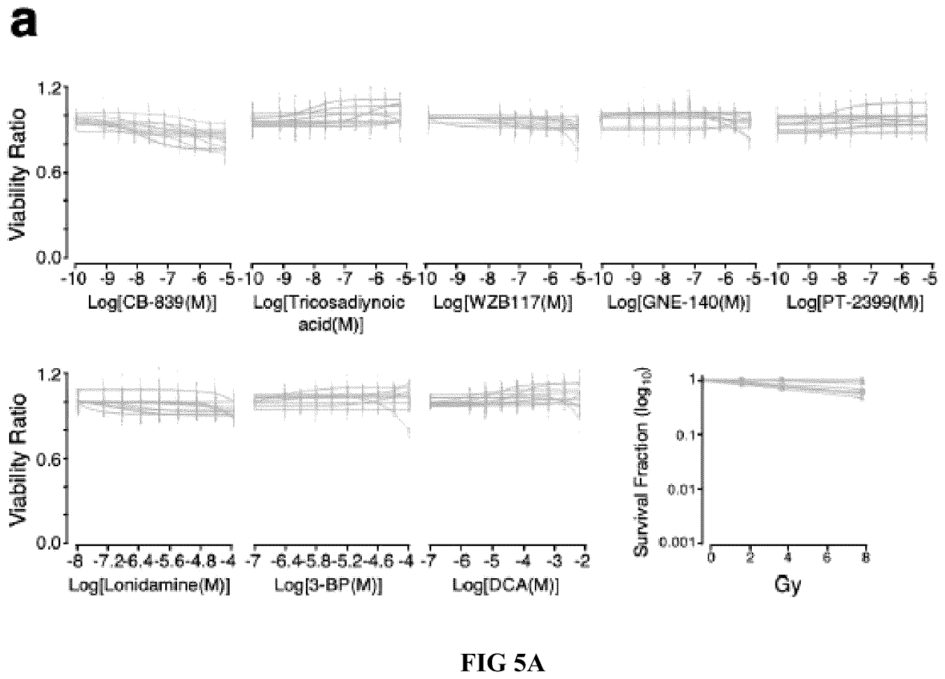

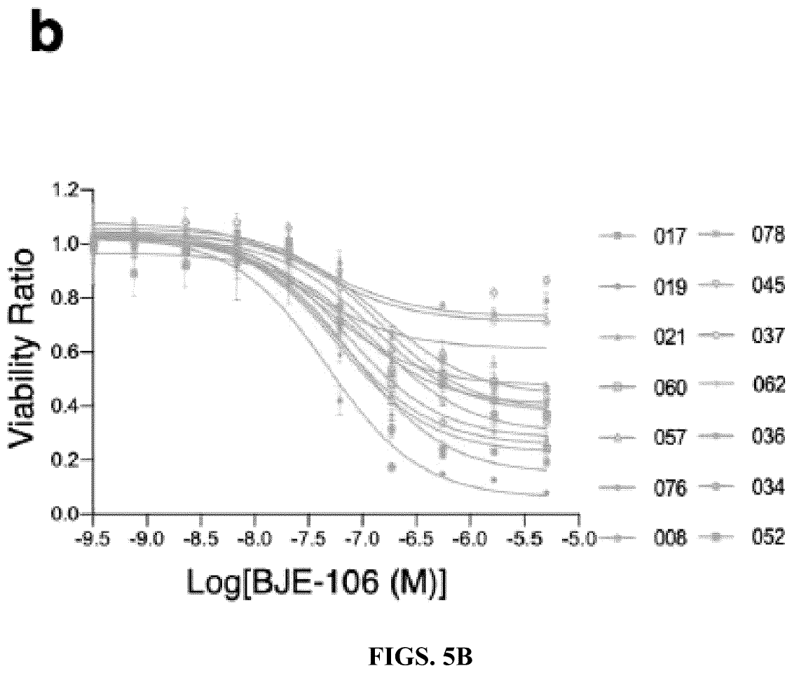

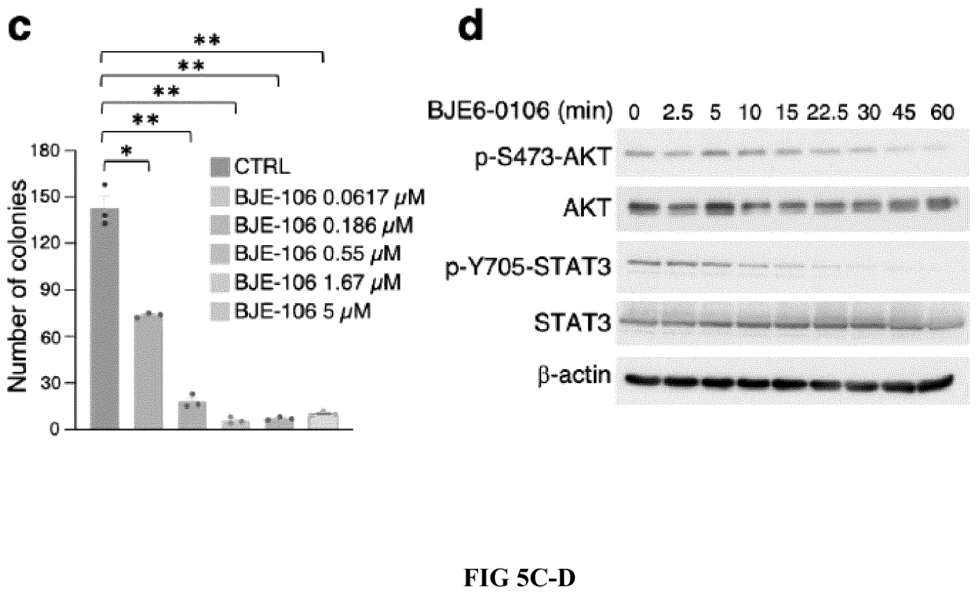

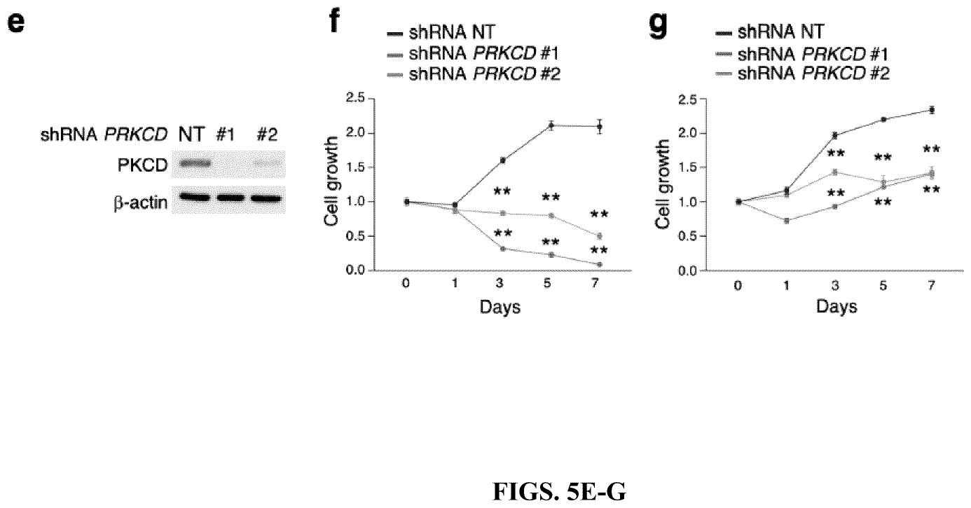

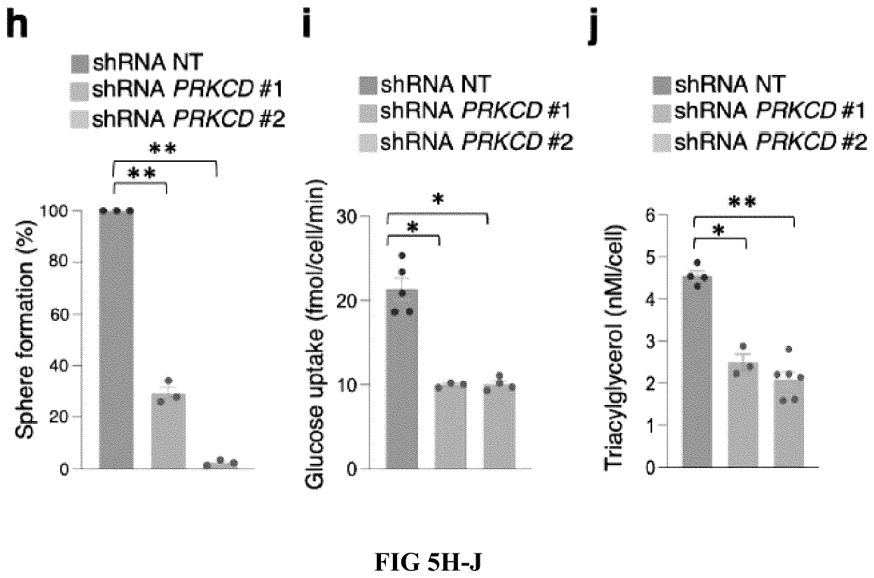

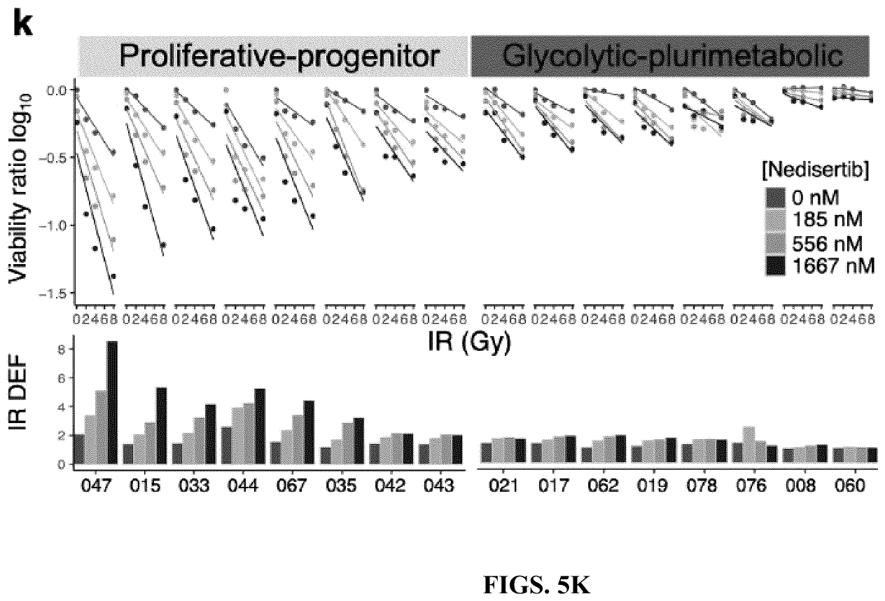

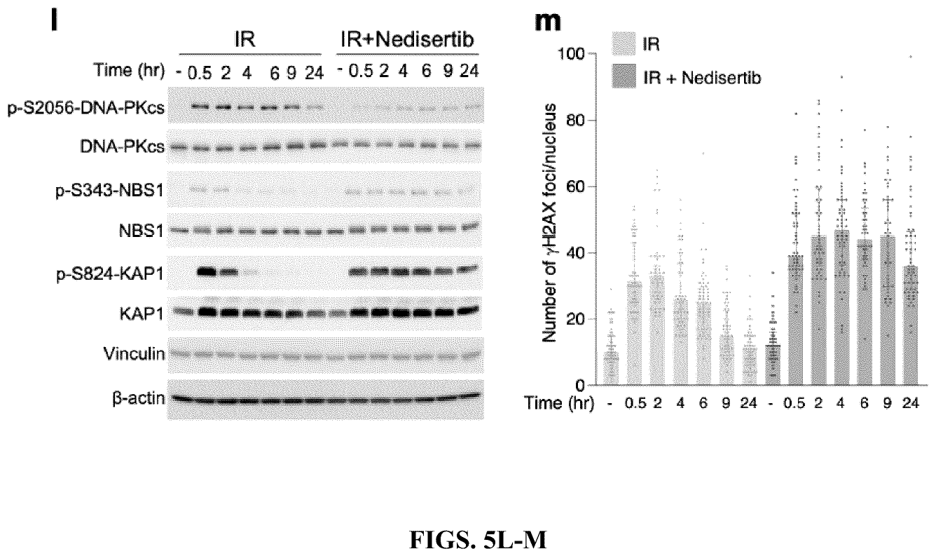

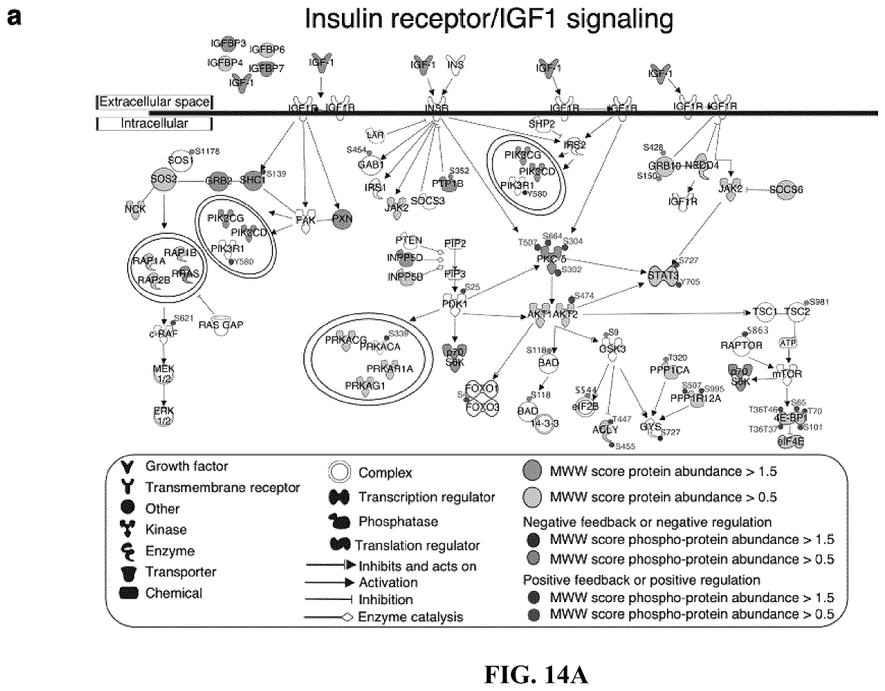

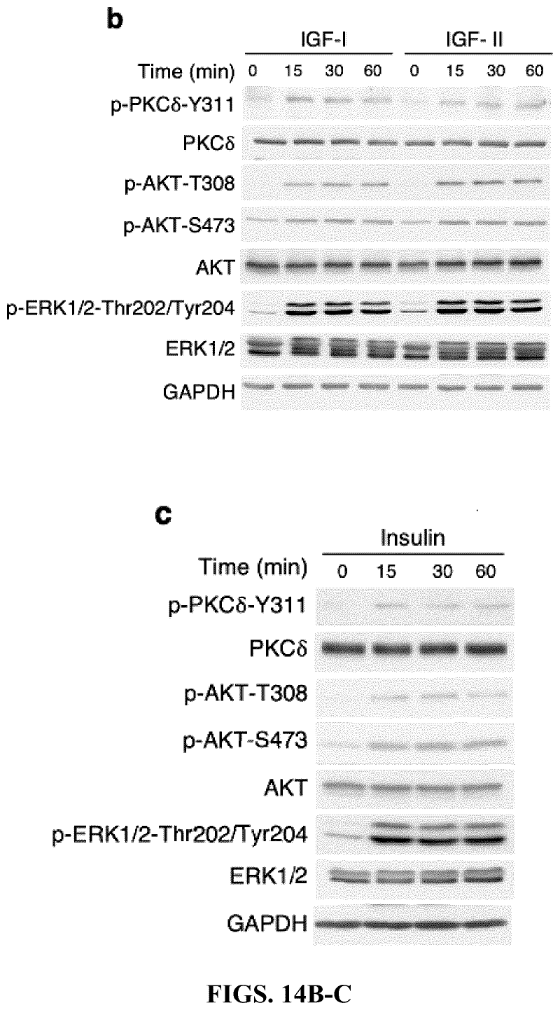

The application of SPHINKS-MK uncovered PKCδ as the top-scoring MK of the GPM subtype. PKCδ controls crucial steps of glucose and lipid metabolism in multiple tissues⁶²⁻⁶⁴. In cancer, PKCδ is a central signaling node of the insulin/IGF/AKT/mTOR signaling pathway that orchestrates the metabolic reprogramming towards aerobic glycolysis and increased uptake of nutrients⁶⁵⁻⁷¹. Activation of PKCδ also mediates resistance to diverse anti-tumor therapies, including therapies that are used with limited success in GBM, such as radiotherapy and inhibitors of receptor tyrosine kinases⁷¹⁻⁷³. Interestingly, recent results suggested that activation of PKCδ may mediate therapy resistance in cancer by upregulation of glucose uptake⁷⁴. As enhanced glucose uptake caused by the elevated expression of glucose transporters, metabolic reprogramming with activation of aerobic glycolysis and lipogenesis, and resistance to radiation are the distinctive hallmarks of the GPM subtype of GBM¹³, we sought to ask whether activation of PKCδ plays a key role in the plurimetabolic phenotype and viability of this GBM subtype. First, we interrogated the sensitivity of GBM PDOs classified as GPM to eight compounds targeting different glycolytic enzymes and confirmed the uniform resistance of this subtype to each of the tested inhibitors. We also found that radiation therapy was ineffective, thus confirming the broad therapeutic resistance of GPM PDOs (FIG. 5a). Next, we asked whether activation of PKCδ in the GPM subtype of GBM is associated with global activation of the insulin/IGF/AKT signaling pathway. Towards this aim, we traced the activation of the pathway by the comparative analysis of protein and phospho-protein abundance of pathway-specific signaling molecules in GPM versus all other GBM subtypes. The analysis indicated that most of the crucial components of the insulin/IGF/AKT pathway are activated in the GPM subtype as indicated by elevation of protein abundance and/or distinct phosphorylation sites, and co-segregate with PKCδ abundance and activation by multiple phosphorylation events (FIG. 14a). Central to the insulin/IGF-PKCδ signaling is the activation of AKT1/2 and STAT3, and both nodes were activated in GPM GBM. Finally, activation of the mTOR kinase (RAPTOR-ser-863) and mTOR substrates (p70S6K, 4E-BP-ser-37/thr-46 phosphorylation) is consistent with the relevance of this pathway for the metabolic reprogramming of the GPM subtype (FIG. 14a). We confirmed experimentally that stimulation by IGF1/2 and insulin induced phosphorylation of PKCδ on tyr-311, a phosphosite crucially required for its activity⁷⁵. As postulated by the proteomics analysis of GPM tumors, activation of PKCδ occurred concurrently with activation of AKT, marked by phosphorylation of thr-308 and ser-473 in GPM PDO cells (FIGS. 14b, c). Finally, we proceeded to interrogate experimentally whether PKCδ activity is essential for cellular fitness and the plurimetabolic state of the GPM subtype. Treatment of GPM PDOs with BJE-10676, a specific third-generation inhibitor of PKCδ, revealed that most of the tested models exhibited marked sensitivity to PKCδ inhibition, with IC50 between 0.1 and 10 M (FIG. 5b). Treatment with BJE-106 also caused dose-dependent inhibition of colony formation (FIG. 5c). Consistent with the identified PKCδ-dependent signaling events, the inhibition of PKCδ activity resulted in time-dependent decrease of AKT-ser-473 and STAT3-tyr-705 phosphorylation (FIG. 5d). To validate the specificity of the effects of pharmacologic inhibition of PKCδ, we performed genetic knock-down of the PRKCD gene in two GPM PDOs using two independent shRNAs for each PDO (FIG. 5e). Quantitative analysis of the proliferation rate and clonogenicity confirmed the requirement of PKCδ for growth and viability of GPM PDOs (FIG. 5f-h). Moreover, functional assessment of the plurimetabolic hallmarks of GPM cells, namely glucose uptake and lipid accumulation¹³ showed that both activities were significantly compromised by silencing of PKCδ (FIG. 5i, j).

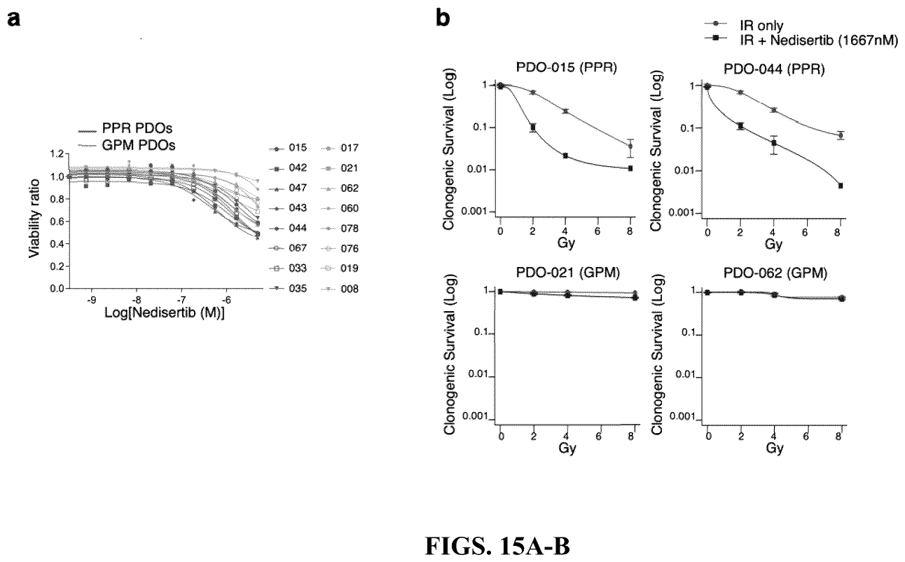

The catalytic subunit of DNA-dependent protein kinase (DNA-PKcs) was among the most active MKs in the PPR subtype of GBM (FIG. 4c, d). DNA-PKcs is one of the three members of PI3K-related kinases (PIKKs) with principal roles in the activation of DDR⁷⁷. DNA-PKcs is activated by multiple types of genotoxic stress, including DNA Double Strands Breaks and RS⁷⁸⁻⁸⁰. Under these conditions, activation of DNA-PKcs is essential to repair the otherwise lethal accumulation of DNA damage. Given the pervasive and specific activation of the DDR and RS in PPR GBM as manifested by multiple analytical platforms (FIGS. 1b, 3a-c, FIG. 11g), we postulated that activation of DNA-PKcs might be required to cope with the increased rates of DNA replication and DDR of the PPR subtype to prevent unsustainable levels of DNA damage. Consequently, we asked whether inhibition of DNA-PKcs with the M3814 (nedisertib) compound, a DNA-PKcs inhibitor currently in clinical studies⁸¹, might promote unique vulnerability of PPR GBM when used in combination with ionizing radiation (IR), the key element in the standard of care for patients with GBM⁸². Treatment of eight PPR GBM PDOs with the nedisertib-IR combination resulted in marked reduction of tumor cell viability compared to each individual treatment, with a radiation dose enhancement factor (DEF) >2 for six of the eight PPR PDOs, even at the lowest concentrations of nedisertib. Conversely, the nedisertib-IR combination was virtually ineffective in eight GPM PDOs, highlighting the specificity of DNA-PKcs inhibition for the PPR subtype of GBM (FIG. 5k, FIG. 15a). We confirmed these results in two PPR and GPM PDOs using the clonogenic assay as a quantitative method to measure radiosensitivity (FIG. 15b). Treatment of PPR GBM with IR caused rapid elevation of the phosphorylation of serine-2056, the key autophosphorylation site of DNA-PKcs marking activation of the kinase^{83,84}. As expected, nedisertib inhibited serine-2056 phosphorylation of DNA-PKcs in irradiated cells (FIG. 5l). Combinatorial treatment also induced persistent, unrepaired DNA damage as indicated by the sustained phosphorylation of serine-343 of NBS1 and serine-824 of KAP1, indicators of active double-strand breaks^{85,86}, which remained stable until 24 hours after irradiation as opposed to the rapid loss of phosphorylation 4 hours after treatment in PDOs that had been exposed to ionizing irradiation alone (FIG. 5l). Consistently, the number of γH2AX foci, which regressed to basal levels in PPR cells treated with irradiation alone, remained elevated throughout the course of the experiment in the presence of DNA-PKcs inhibition (FIG. 5m).

Pediatric and Adult Cancers Cluster into Functionally Conserved Subtypes that Share MK Activation

In an effort to determine whether the key biological functions discriminating the GBM subtypes coalesce into grouping patterns sharing the same kinase-driven dependencies, we first determined whether a functional classification of other tumor types could be obtained. We focused on three different cancer types, Pediatric Glioma (PG)⁸⁷ that arise in the brain as for GBM, Breast Carcinoma (BRCA) and Lung Squamous Cell Carcinoma (LSCC) for which genomics, proteomics and phosphoproteomics datasets are available⁹⁻⁸⁸.

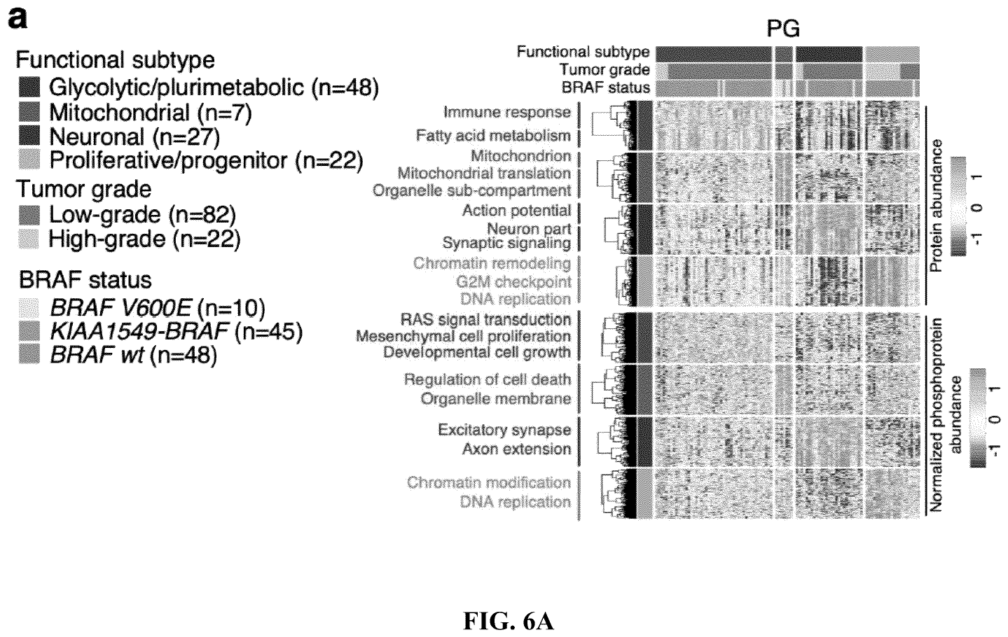

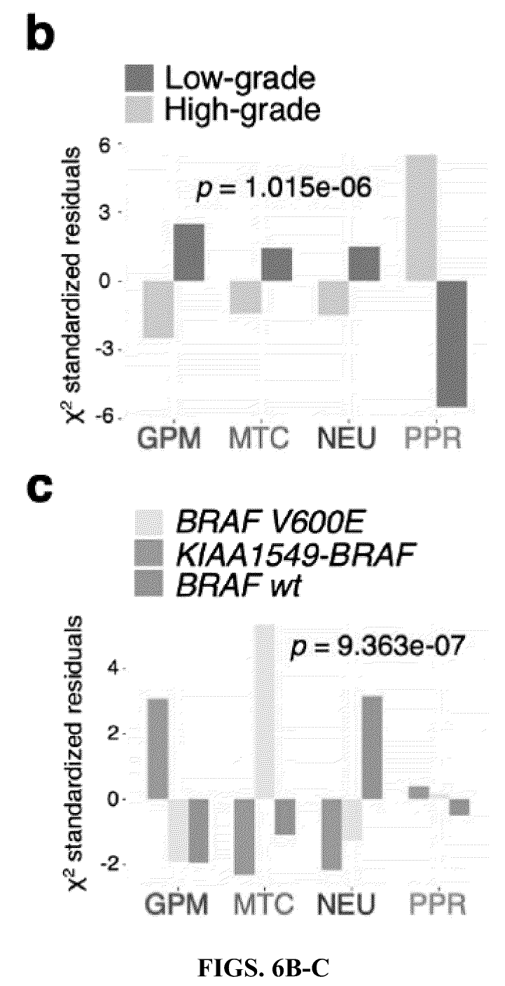

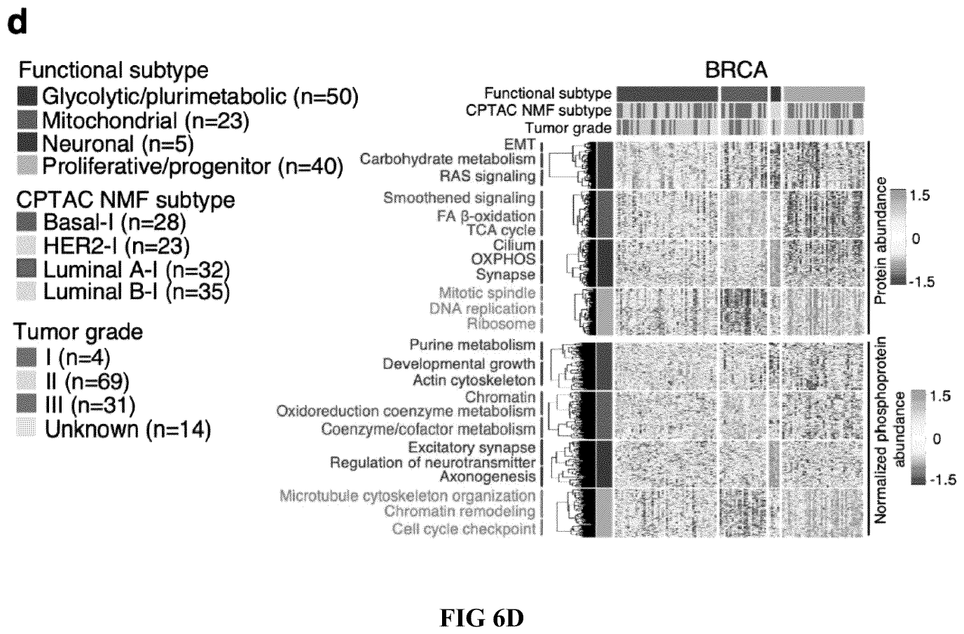

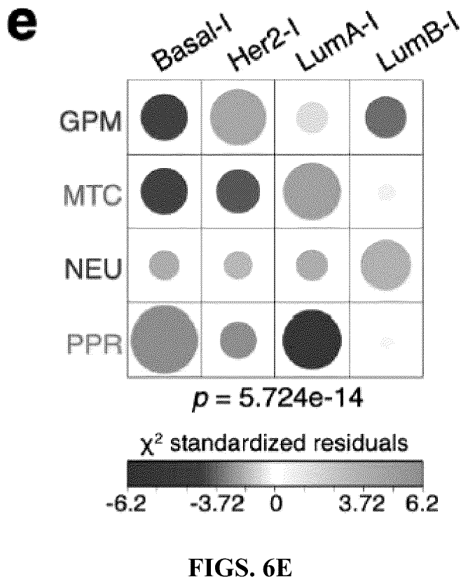

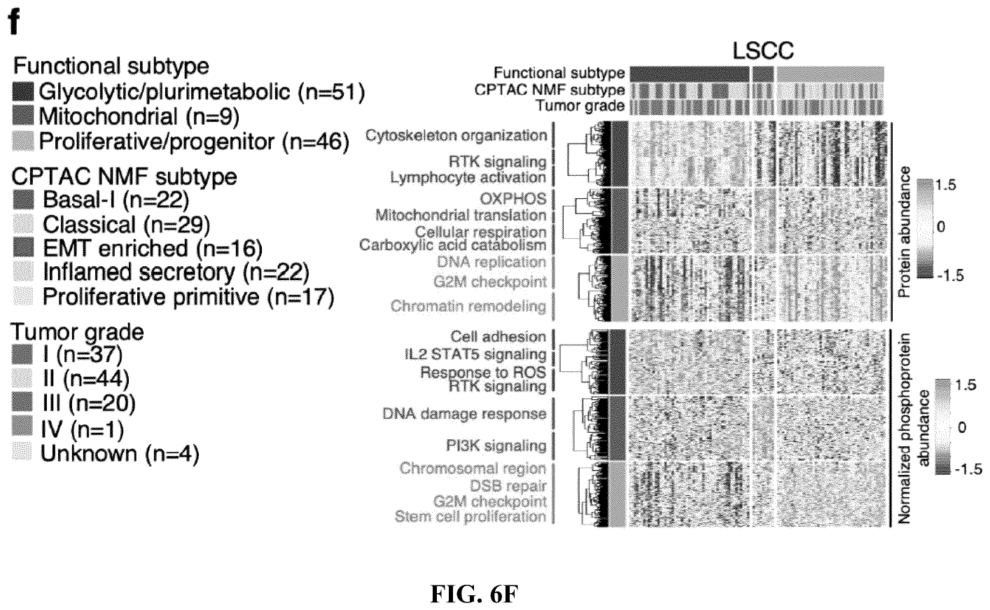

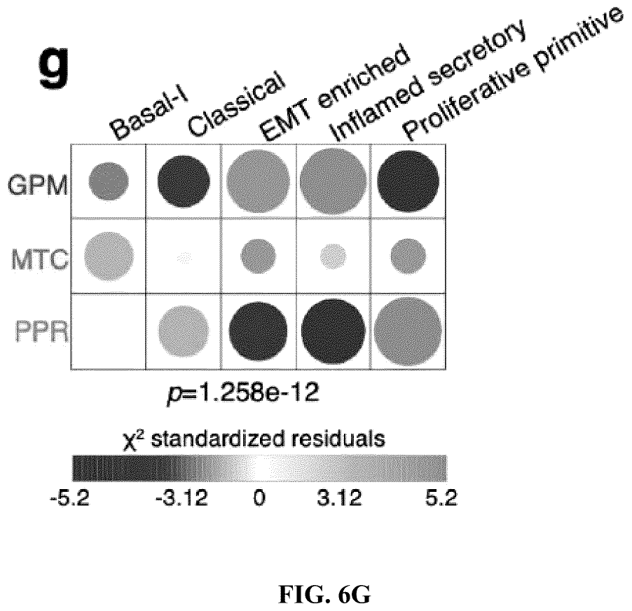

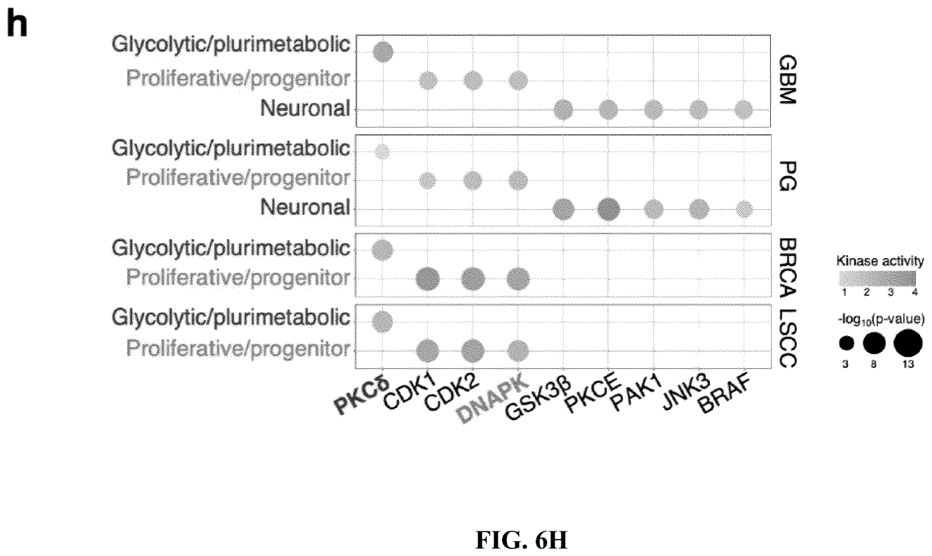

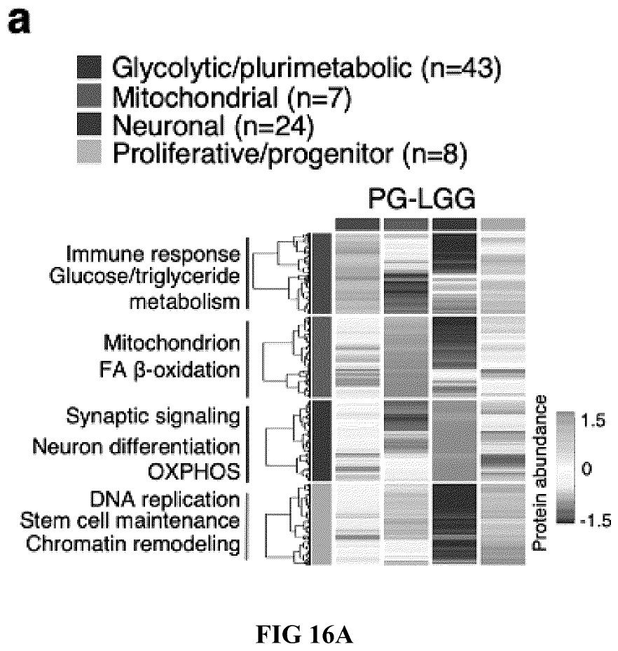

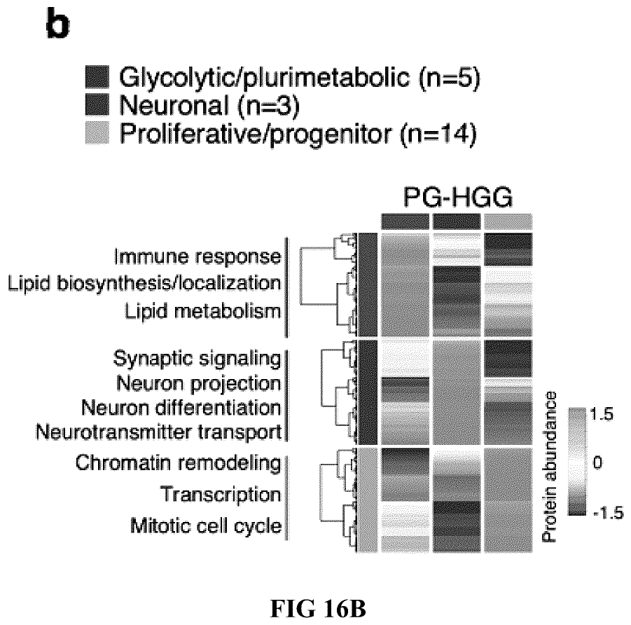

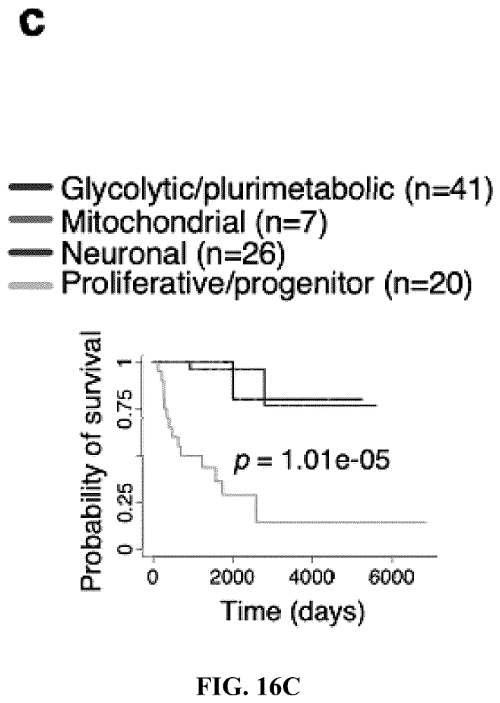

For PG, we integrated protein and gene expression data of 103 samples classified as high-grade (PG-HGG) or low-grade (PG-LGG) gliomas using the SNF approach as implemented for the classification of GBM. We identified four subtypes of PG, each characterized by the predominant biological activities that define the functional classification of GBM, at proteomic, phosphoproteomic and gene expression level (GPM, MTC, PPR and NEU, FIG. 6a). PG-HGG mostly clustered within the PPR subtype whereas PG-LGG distributed across each of the four subgroups (FIGS. 6a, b). When PG-HGG and PG-LGG were analyzed independently for differential protein abundance, high- and low-grade tumors clustered into three and four groups, respectively, with the MTC subtype excluded from PG-HGG (FIGS. 16a, b). Genetic alterations of BRAF are common in PG-LGG⁸⁹. The KIAA1549-BRAF fusion is the most frequent alteration in PG-LGG (35%) and it is almost exclusively a single-driver event in these tumors⁹⁰. Conversely, the BRAF-V600E mutation, which is the second most common alteration in PG-LGG (17%), is frequently associated with additional genetic alterations⁹¹. Glioma harboring the BRAF-V600E mutation were mostly classified as MTC (five of seven MTC tumors were V600E mutated) whereas PG-LGG harboring the KIAA1549-BRAF fusion were enriched with GPM and BRAF wild type PG-LGG with NEU tumors (FIGS. 6a, c). Kaplan-Meier and log-rank test demonstrated significantly worse survival for the PPR subtype, a finding compatible with the predominant contribution of high-grade tumors to this group (FIG. 16c).

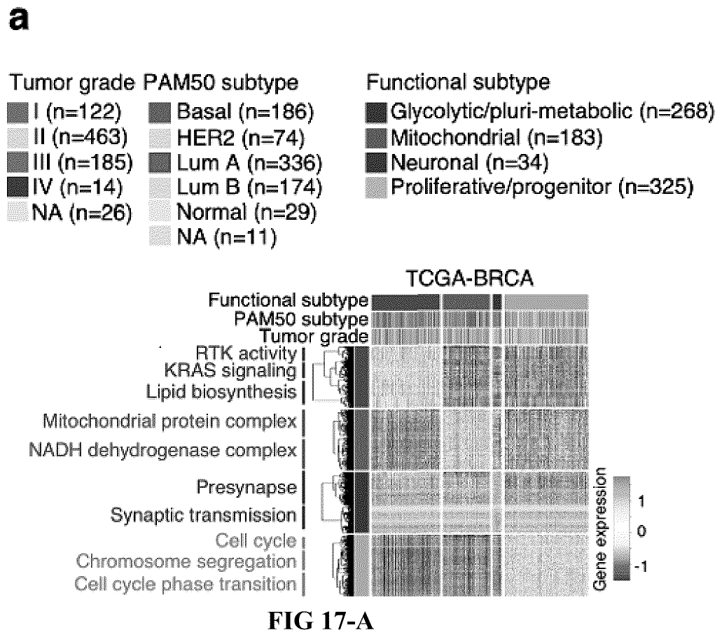

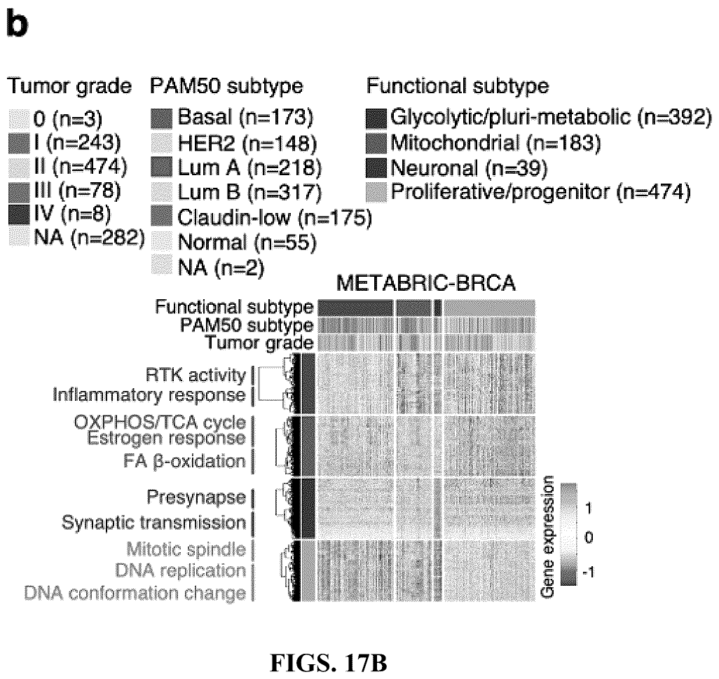

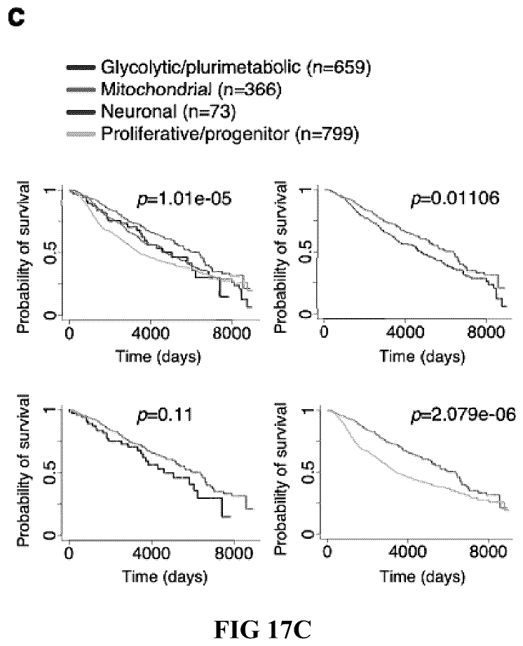

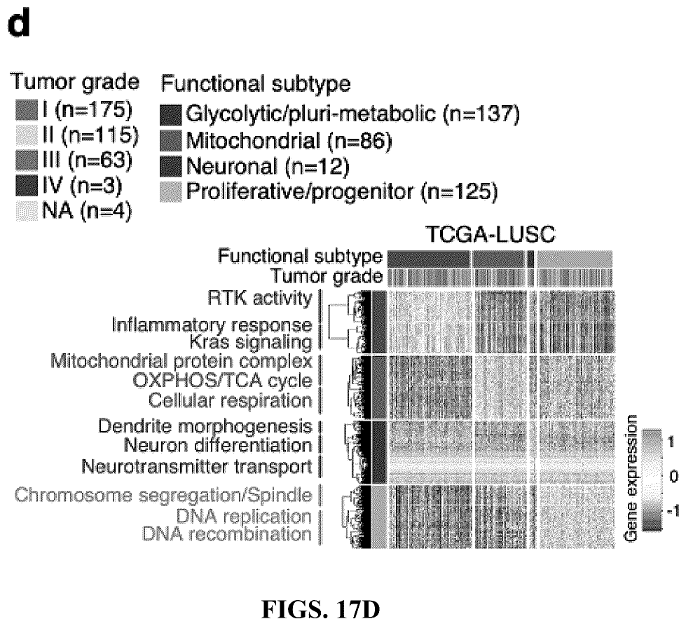

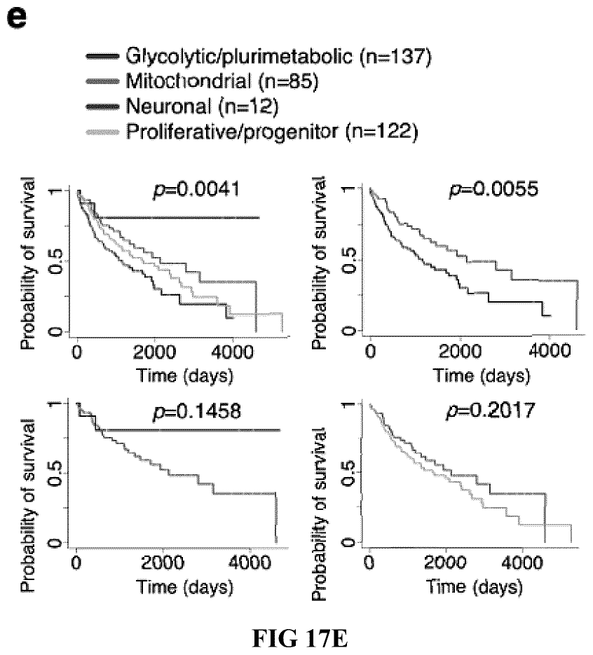

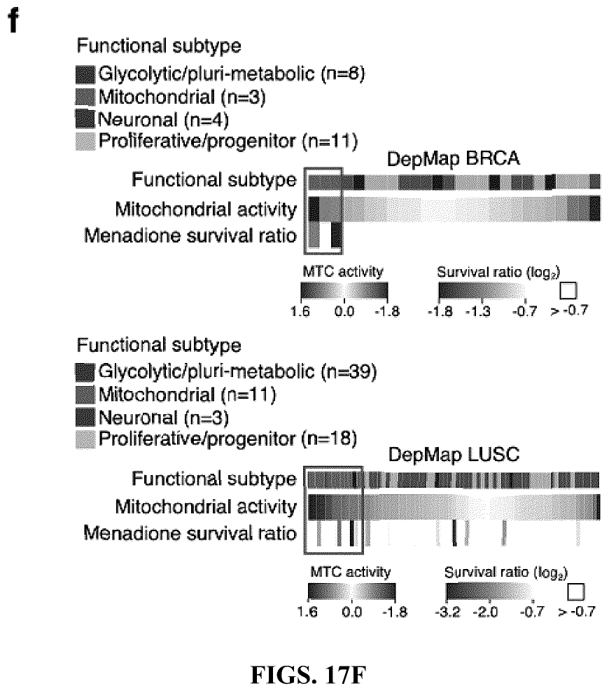

We also classified 118 BRCA samples into four subtypes having coherent gene expression, protein and phosphoprotein abundance signatures, three major groups representing 95% of the samples (GPM, PPR and MTC) and one smaller group, the NEU subgroup, including only 5 tumors (FIG. 6d). We compared these functional subtypes with subtypes reported by the CPTAC and found a striking association of the HER2-I (I, inclusive as defined by integrative CPTAC analysis) subgroup with the GPM subtype, Basal-I with PPR, LumA-I with MTC and LumB-I with NEU (FIG. 6e). The enrichment of HER2-I in the GPM subtype is consistent with hyperactivation of the mTOR pathway and a metabolic shift from aerobic respiration to glycolysis in this BRCA subtype⁹². The stability of the functional classification of BRCA was verified using TCGA and METABRIC gene expression data, thus authenticating the biological activities as general features for BRCA categorization (FIGS. 17a, b). We also found significantly better prognosis associated with the MTC-BRCA subtype, which is consistent with the prolonged survival of LumA-I (FIG. 17c). The positive association between PPR and Basal-I subtype was further supported⁵ by the strong enrichment of DNA replication and proliferation-associated pathways in the Basal-I subtype of BRCA (FIG. 6d).

Finally, we used the functional classifier to segregate a cohort of 106 LSCC tumors and tested the association with the five known LSCC-specific molecular NMF-based subtypes (FIGS. 6f, g). LSCC tumors were classified into two major subtypes (GPM and PPR) and a much smaller MTC subgroup. In this limited dataset we did not identify NEU tumors. We found a positive correlation of the MTC subtype with the basal-I subgroup. EMT and inflamed secretory LSCC subtypes described by CPTAC as two independent groups were functionally unified by the activation of immune, EMT and angiogenesis functions of the GPM subtype. The PPR subtype of LSCC included proliferative-primitive and classical subtypes, both sustained by proliferative-related pathways (FIGS. 6f, g)^{88,93}. The robustness of the functional subtyping was validated in the TCGA-LUSC (Lung squamous carcinoma) datasets using gene-expression signatures derived from the analysis of the CPTAC cohorts (FIG. 17d). In this larger cohort, 12 tumors exhibited activation of synaptic functions, hallmark of the NEU subtype. MTC-LSCC tumors exhibited more favorable clinical outcome, suggesting that also in this tumor type OXPHOS activation produces a less aggressive biology and/or increases sensitivity to therapy (FIG. 17e)¹³. Dependency of the MTC subtype of BRCA and LUSC on mitochondrial activity was supported by the association between MTC activity (NES) of BRCA and LUSC cell lines in the DepMap dataset⁹⁴ and sensitivity to menadione, a cytotoxin that specifically targets mitochondria (FIG. 17f)⁹⁵.

Having stratified PG, BRCA and LSCC on the basis of a functional classification, we applied SPHINKS to each proteome/phosphoproteome dataset and generated three distinct tumor-specific kinase-phosphosite interactomes. The SPHINKS interactomes included 669, 1,399 and 1,985 kinase-phosphosite relationships that originated from 76, 198, and 103 kinases and 210, 1,899, and 699 phosphosites for PG, BRCA and LSCC, respectively. The three kinase-phosphosite interactomes were used to identify tumor subtype-specific MKs by applying the MWW test [log 2(FC)>0.3, p<0.01]. MKs were finally validated across multiple platforms including global protein abundance and mRNA expression. Nine protein kinases emerged as top ranking MKs consistently activated in the same functional subtypes of GBM, PG, BRCA and LSCC. Among them, PKCδ scored as pan-GPM and DNA-PKcs as pan-PPR MKs (FIG. 6h). In fact, PKCδ was the only MK consistently linked to all the GPM tumor subtypes across the analyzed tumor types, thus qualifying as key hub for the glycolytic/plurimetabolic state of cancer cells, regardless of the cell of origin. DNA-PKcs emerged as the only DDR-associated PPR-MK in all tumor types, therefore providing unique multi-cancer therapeutic opportunities targeting DNA-PKcs in this functional subtype.

### Development of a Probabilistic Functional Classifier of GBM

We designed an algorithm for the probabilistic classification of individual tumors into one of the four functional GBM subtypes. We generated two different classifiers, one informed by RNA-Seq data of fresh frozen tumor samples (“frozen model”) and the other by RNA-Seq data from formalin-fixed paraffin-embedded (FFPE) tumors (“FFPE model”). The dual approach was motivated by the notion that, when compared with RNA derived from fresh frozen samples, FFPE-extracted RNA is characterized by lower quality, typically affecting different mRNA species to variable extent⁹⁶⁻⁹⁸.

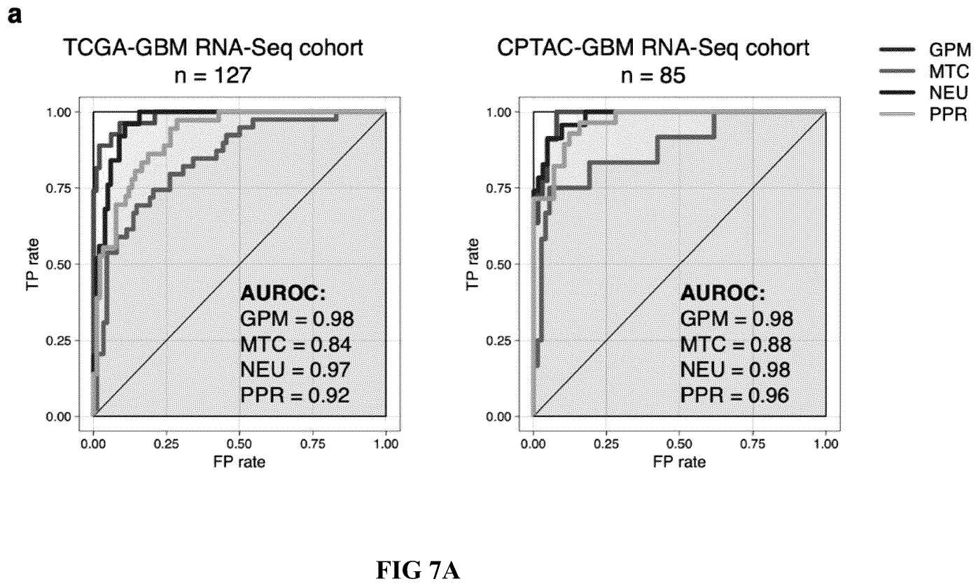

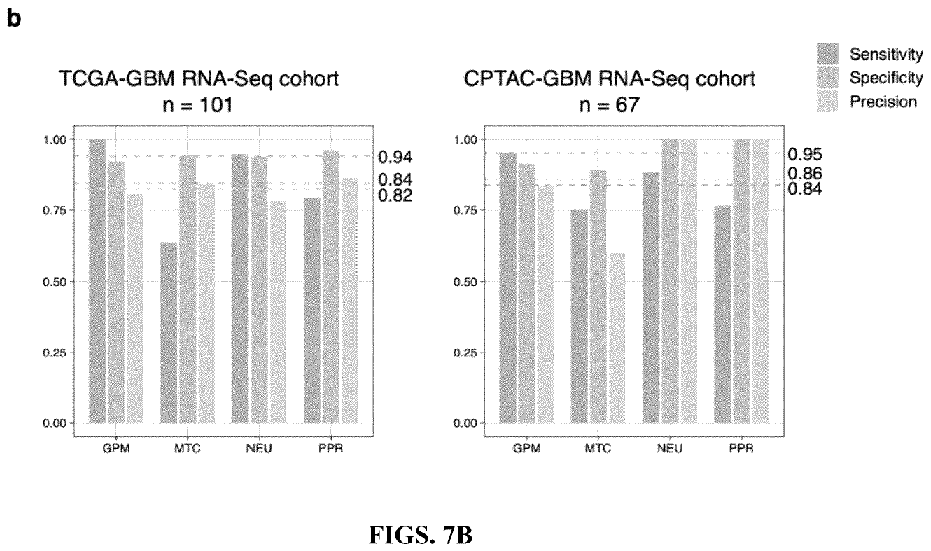

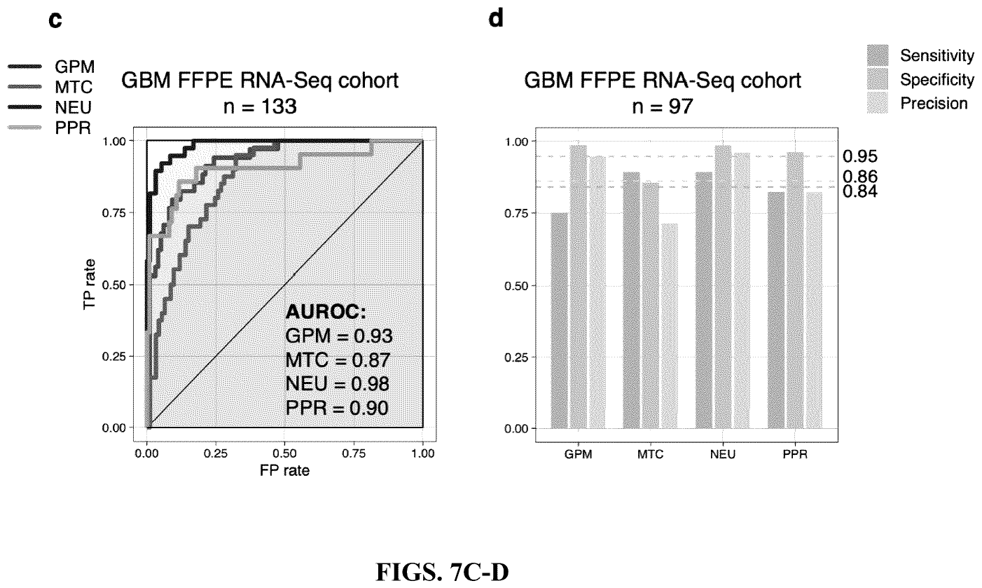

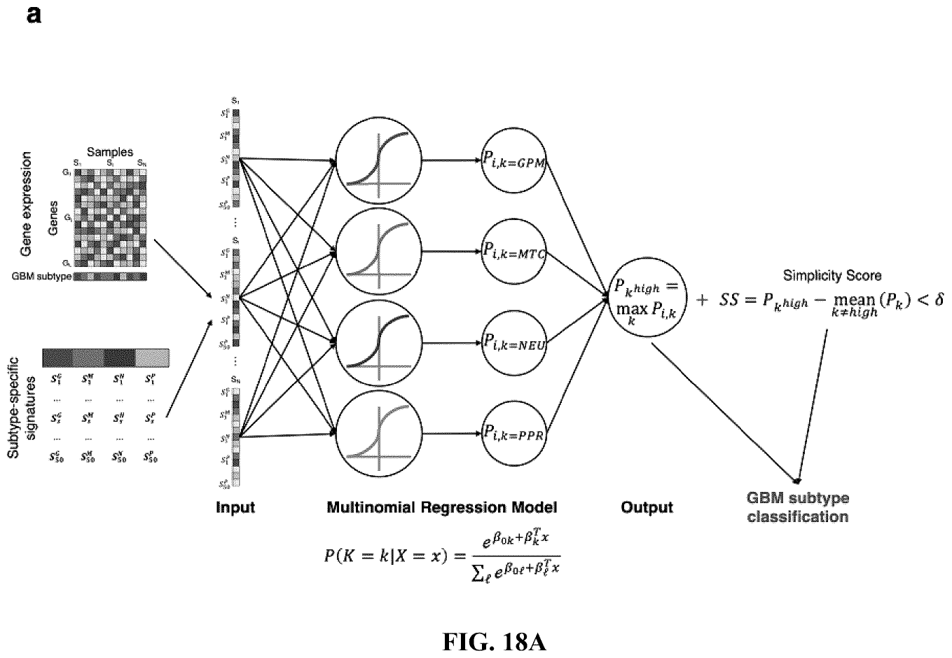

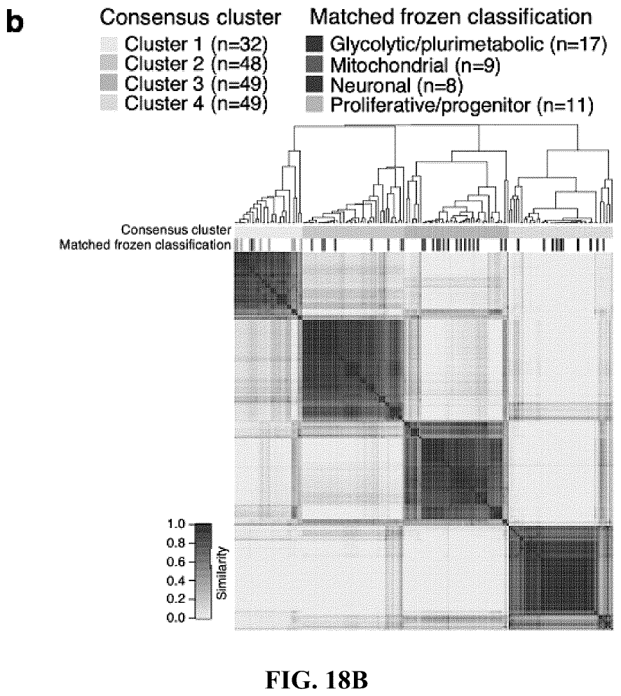

For the frozen model, we trained the classifier using the multinomial regression model with lasso penalty⁹⁹⁻¹⁰¹ and the TCGA IDH wild type GBM dataset profiled by Agilent expression arrays, which we had classified in previous work (FIG. 18a)¹³. As starting feature set, we selected the expression of the 50 highest ranking genes of each functional subtype in the TCGA cohort, for a total of 200 gene features¹³. To extract a reduced set of features that maximize the distinctiveness of the phenotypes, we applied a cross-validation approach and selected the model exhibiting the lowest misclassification error (17.19% cross-validation error and 6.32% error on the training set). The models that emerged from this step indicated a set of 103 gene features with both positive and negative coefficients, generally coherent with the biology assigned to each GBM subgroup. We classified a tumor sample into one particular subtype when the fitted probability of that subtype was the highest and the sample had a simplicity score above a predefined threshold (see Methods). Samples that did not comply with the defined thresholds, likely harboring extensive intratumor heterogeneity, were left unclassified. We tested the prediction ability of the “frozen classifier” using two independent validation cohorts, including 127 GBM from TCGA and 85 GBM in the CPTAC cohort both profiled by RNA-Seq. We classified 80% and 79% of the TCGA and CPTAC GBM, respectively. We evaluated the diagnostic ability of the classifier system by deriving the area under the receiver operating characteristic (AUROC) curve of each subtype (FIG. 7a). Remarkably, subtype-specific AUROCs were above 0.85 in each validation dataset, with 1 indicating perfect classification. Finally, we determined the accuracy of the assignment of each tumor to the correct subtype by calculating misclassification error, subtype-specific sensitivity, specificity and precision for each validation dataset¹⁰². The misclassification error was below 18% for the considered validation sets and the sensitivity consistently approached 85%. Specificity and precision were close to 100% and above 80%, respectively, indicating robust performance of the classifier (FIG. 7b).

For the FFPE model, we used an approach similar to the one applied to samples profiled by RNA-Seq from frozen tissues with some modifications to account for the lower quality of FFPE-extracted RNA. We sequenced the transcriptome of 45 matched frozen and FFPE samples and defined a set of 4,673 genes that exhibited consistent expression profiles in fresh frozen and FFPE-derived RNA (genes supposedly unaffected by FFPE treatment, Spearman correlation, p>0.22). Considering the classification of the 45 matched frozen samples as gold standard, we used the expression profiles of the corresponding FFPE samples to generate new subtype-specific signatures. We then trained the multinomial regression model with lasso penalty using the expression of the FFPE-specific signature genes from the TCGA IDH wild type GBM profiled by Agilent expression arrays (19.76% cross-validation error, 11.07% error on the training set, and 66 gene features). To evaluate the performance of the classifier, we profiled the transcriptome of an independent cohort of 133 FFPE GBM samples by RNAseq. By applying the “FFPE model” as done for the “frozen model”, we classified 73% of the samples. To assess the stability and accuracy of the “FFPE model”, we sought to unbiasedly assign FFPE samples to a GBM subtype using FFPE-specific gene expression signature. Unsupervised consensus clustering of 178 GBM samples including 133 GBM FFPE and 45 samples that had RNAseq data from matched FFPE and frozen tissue revealed four robust and stable clusters (FIG. 18b). Using the classification of the 45 matched frozen samples as “anchors”, we were able to assign each individual cluster to a distinct functional GBM subtype. Finally, for each of the 133 FFPE samples, we matched the subtype classification from the “FFPE model” with the unbiased label assignment obtained from consensus clustering. The classifier performance indexes were similar to those calculated for the “frozen model” (misclassification error of 15%, AUROCs, sensitivity, specificity and precision above 0.84) (FIGS. 7c, d). We have implemented a Shiny app of the frozen and FFPE classification tools for general research use at lucgar88.shinyapps.io/GBMclassifier.

The proteogenomic and multi-omics profiling of several human tumors generated a comprehensive catalogue of molecular features that have mostly been associated with cancer-driving genetic alterations and well-established pathologic and molecular tumor subtypes. From these studies potential therapeutic targets have emerged, typically linked to oncogenic drivers confirmed by proteogenomics. However, the lack of experimental follow-up in relevant cancer models that faithfully recapitulate distinct tumor subtypes leaves the proposed targets unvalidated. Here, we sought to establish a link between the orthogonal multi-omic features that regulate the biology of GBM subtypes and protein kinases that could directly enable subtype-specific phenotypes. We built and applied SPHINKS-MK, an algorithm that integrates proteomics and phosphoproteomics datasets into a single network for the unbiased extraction of the MKs of tumor subtypes. By informing pharmacologic and genetic experiments in subtype-specific GBM organoids, SPHINKS-MK delivered PKCδ and DNA-PKcs as experimentally validated MKs for the aggressive GPM and PPR subtypes of GBM. The four subtypes and the underlying phenotypes were also recovered across different tumor types, highlighting the fundamental biological traits that are extracted by the functional classification. In the multi-cancer context, PKCδ and DNA-PKcs have emerged as broadly actionable MKs of GPM and PPR subtypes, regardless of the tissue of origin. Inspired by the subtype-specific therapeutic opportunities, we present a probabilistic classifier that enables rapid translation of precision therapeutics for stratified subgroups of GBM patients.

The four GBM subtypes initially inferred from a pathway-based scRNAseq analysis are supported by orthogonal features from proteomics, phosphoproteomics, metabolomics, lipidomics and acetylomics platforms. The divergent metabolism of the GPM and MTC subtypes was independently captured by the analysis of acetylomics, a post-translational modification previously associated with the inactivation of metabolic proteins³⁹. Whereas the GPM subtype was enriched with the acetylation of mitochondrial proteins and OXPHOS enzymes, the MTC subtype was enriched with acetylation of proteins implicated in multiple metabolic circuits that include glycolysis, gluconeogenesis, amino acids and lipid biosynthesis but not mitochondrial metabolism. Acetylation has also emerged as major determinant factor instructing the identity of the proliferation-, stemness- and DDR-related biology that is activated in PPR cells. Stratification of PPR-GBM based on acetylation of nuclear proteins uncovered a hyperacetylated PPR group of tumors with outlier activation of these activities. This finding is consistent with the notion that acetylation is a crucial regulatory modification of nuclear proteins, implicated in the activation of transcription and chromatin-remodeling factors, and enzymes involved in the DDR¹⁰³.

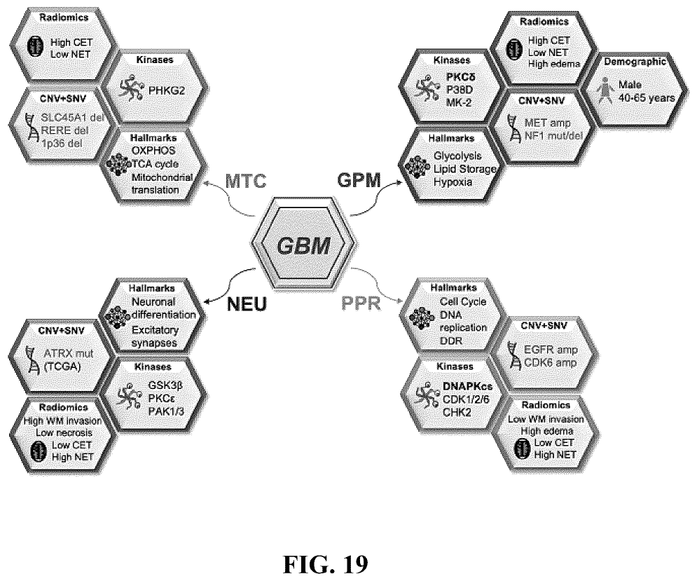

The significance of the pathway-based classification of GBM is further underscored by the association of the individual subtypes with clinical variables such as age and tumor location within the CNS, and frequency of recurrent alterations of driver genes. By interrogating the MR imaging features associated with each subtype, we uncovered that the metabolic subtypes, and particularly the GPM subgroup, are characterized by high degree of contrast enhancement, potentially reflecting more prominent perivascular invasion of tumor cells with consequent disruption of the endothelial tight junctions of the blood-brain barrier. Conversely, tumors classified along the neurodevelopmental axis are consistently associated with non-enhancing features. Among them, the unique correlation between NEU tumors and deep white matter invasion is consistent with the proposed ability of neuronally differentiated GBM cells to engage normal brain cells at the tumor periphery for neomorphic synaptic connections that guide invasion through white matter tracks¹³. Taken together, the pathway-based classification of GBM was orthogonally supported by biologically coherent multi-omic features and clinical variables that we summarized into an illustration conveying the most significant marks of each subtype (FIG. 19).

Before this work, the accurate prediction of protein kinase activity in individual tumor samples remained underdeveloped. Predictive models attempted to use the abundance and/or state of phosphorylation of kinase proteins as proxy for their activity¹⁰⁴. Alternatively, kinase activity has been predicted by methods based on the limited set of experimentally validated kinase-substrate pairs, irrespective of the complexity of tissue-specific regulatory networks that generate the specificity of the protein kinases-substrates interactome (kinase regulons)⁸⁷. To reduce the complexity of coexisting transcription factors and target genes within the same transcriptomic interactome, we previously developed computational approaches for the reconstruction of accurate transcriptomic networks^{13,56,105}. While we found that activation of handful transcription factors was sufficient to activate the gene expression signatures of particular tumor subtypes, direct therapeutic targeting of transcription factors remains challenging¹⁰⁶. Conversely, there is tremendous appeal of protein kinases as both drivers and drug targets, with 62 FDA-approved kinase inhibitors currently available for precision therapeutics of cancer patients¹⁰⁷. The SPHINKS-MK algorithm interrogates the full scope of any tumor-specific kinome and phosphorylome assembled into an integrated functional network to identify high-activity kinases specific for each tumor subtype. The benchmark of SPHINKS showed that the algorithm was stable despite significant perturbations of the datasets. It also demonstrated prediction power higher than other inference methods.

Among functional GBM subtypes, the PPR and GPM subtypes include the most frequent and aggressive tumors. The dismal prognosis of patients affected by these GBM subtypes instigated follow-up experimentation using subtype-matched GBM-PDO models. PKCS emerged as the top-scoring kinase in the GPM subtype. Genetic and pharmacologic inhibition of PKCδ defined its role in oncometabolic processes at the intersection of insulin, IGF, and lipid metabolism and validated PKCδ as crucial therapeutic target in the GPM subtype of GBM. For the PPR subtype, DNA-PKcs, one of the three members of the family of phosphatidylinositol 3-kinase related kinases (PIKKs) with principal role in activating various forms of DDR, was experimentally validated as the essential MK⁷⁷. The striking synergistic and lethal effect of inhibition of DNA-PKcs and irradiation in PPR cells but not in GPM provided the mechanistic interpretation of therapy resistance in this GBM subtype. As DNA-PKcs inhibitors have been introduced into clinical trials⁸¹⁻¹⁰⁸, our findings support the notion that pre-selection of patient with PPR tumors is likely to enhance therapeutic success. Having generated and validated the GBM-specific kinase-phosphorylome network, any new GBM sample profiled by phosphoproteomics can now be analyzed to extract patient-specific MKs and construct a kinase inhibitor efficacy map for individual GBM. The GBM classifier was orthogonally validated to stratify pediatric and adult tumors, revealing consistent patterns across different tumor types (e.g. favorable survival associated with MTC tumors) and context-dependent features (BRAF mutations and fusions associated with divergent metabolic subtypes in PG). The consistent identification of PKCδ and DNA-PKcs as top scoring subtype-specific MKs from the SPHINKS kinase-phosphosite interactomes independently built from the PG, BRCA and LSCC data provides targeted therapeutic opportunity against the GPM and PPR subtypes across multiple tumor types.

Together with our ability to target MTC GBM with OXPHOS inhibitors¹³, the multi-omics classification presented here delivers experimentally validated precision targeting opportunities for the functional tumor subtypes. To implement the GBM classifier in clinical settings, we developed a probabilistic classification tool for the translation of diagnostic and therapeutic information emerging from this work. The classifier was cross-validated in multiple datasets and independently optimized for fresh frozen and more importantly FFPE tumor specimens, the most frequently available diagnostic material in the clinical pathology setting. The classifier will facilitate the yet unfulfilled stratification of patients with GBM for the accrual to clinical trials and accelerate the development of precision therapies targeting individual subtypes of this aggressive tumor.

### REFERENCES FOR EXAMPLE 1

1 Simon, R. & Roychowdhury, S. Implementing personalized cancer genomics in clinical trials. Nat Rev Drug Discov 12, 358-369 (2013).
2 Marshall, K. et al. Quantitative trait loci for resistance to Haemonchus contortus artificial challenge in Red Maasai and Dorper sheep of East Africa. Anim Genet 44, 285-295 (2013).
3 Kundra, R. et al. OncoTree: A Cancer Classification System for Precision Oncology. JCO Clin Cancer Inform 5, 221-230 (2021).
4 Stahl, E. L. & Bohn, L. M. Decaf or regular? Energizing the caffeine receptor. Cell 184, 1659-1660 (2021).
5 Zhang, H. et al. Integrated Proteogenomic Characterization of Human High-Grade Serous Ovarian Cancer. Cell 166, 755-765 (2016).
6 Mertins, P. et al. Proteogenomics connects somatic mutations to signalling in breast cancer. Nature 534, 55-62 (2016).
7 Zhang, B. et al. Proteogenomic characterization of human colon and rectal cancer. Nature 513, 382-387 (2014).
8 Dou, Y. et al. Proteogenomic Characterization of Endometrial Carcinoma. Cell 180, 729-748 e726 (2020).
9 Krug, K. et al. Proteogenomic Landscape of Breast Cancer Tumorigenesis and Targeted Therapy. Cell 183, 1436-1456 e1431 (2020).
10 Su, K. et al. Pan-cancer analysis of pathway-based gene expression pattern at the individual level reveals biomarkers of clinical prognosis. Cell Rep Methods 1 (2021).
11 Mateo, L. et al. Personalized cancer therapy prioritization based on driver alteration co-occurrence patterns. Genome Med 12, 78 (2020).
12 Ben-Hamo, R. et al. Predicting and affecting response to cancer therapy based on pathway-level biomarkers. Nat Commun 11, 3296 (2020).
13 Garofano, L. et al. Pathway-based classification of glioblastoma uncovers a mitochondrial subtype with therapeutic vulnerabilities. Nature Cancer 2, 141-156 (2021).
14 Wang, L. B. et al. Proteogenomic and metabolomic characterization of human glioblastoma. Cancer Cell 39, 509-528 e520 (2021).
15 Wang, B. et al. Similarity network fusion for aggregating data types on a genomic scale. Nat Methods 11, 333-337 (2014).
16 Nagaraj, N. et al. Deep proteome and transcriptome mapping of a human cancer cell line. Mol Syst Biol 7, 548 (2011).
17 Vogel, C. & Marcotte, E. M. Insights into the regulation of protein abundance from proteomic and transcriptomic analyses. Nat Rev Genet 13, 227-232 (2012).
18 Payne, S. H. The utility of protein and mRNA correlation. Trends Biochem Sci 40, 1-3 (2015).
19 Wang, Q. et al. Tumor Evolution of Glioma-Intrinsic Gene Expression Subtypes Associates with Immunological Changes in the Microenvironment. Cancer Cell 32, 42-56 e46 (2017).
20 Capper, D. et al. DNA methylation-based classification of central nervous system tumours. Nature 555, 469-474 (2018).
21 Bielle, F. et al. Diffuse gliomas with FGFR3-TACC3 fusion have characteristic histopathological and molecular features. Brain Pathol 28, 674-683 (2018).
22 Gutman, D. A. et al. MR imaging predictors of molecular profile and survival: multi845 institutional study of the TCGA glioblastoma data set. Radiology 267, 560-569 (2013).
23 Jain, R. et al. Outcome prediction in patients with glioblastoma by using imaging, clinical, and genomic biomarkers: focus on the nonenhancing component of the tumor. Radiology 272, 484-493 (2014).
24 Varn, F. S. et al. Glioma progression is shaped by genetic evolution and microenvironment interactions. Cell 185, 2184-2199 e2116 (2022).
25 Molenaar, M. R. et al. LION/web: a web-based ontology enrichment tool for lipidomic data analysis. Gigascience 8 (2019).
26 Park, M. et al. A Role for Ceramides, but Not Sphingomyelins, as Antagonists of Insulin Signaling and Mitochondrial Metabolism in C2C12 Myotubes. J Biol Chem 291, 23978-23988 (2016).
27 Petan, T., Jarc, E. & Jusovic, M. Lipid Droplets in Cancer: Guardians of Fat in a Stressful World. Molecules 23 (2018).
28 Zigdon, H. et al. Ablation of ceramide synthase 2 causes chronic oxidative stress due to disruption of the mitochondrial respiratory chain. J Biol Chem 288, 4947-4956 (2013).
29 Carrasco, S. & Merida, I. Diacylglycerol, when simplicity becomes complex. Trends Biochem Sci 32, 27-36 (2007).
30 Heden, T. D. et al. Mitochondrial PE potentiates respiratory enzymes to amplify skeletal muscle aerobic capacity. Sci Adv 5, eaax8352 (2019).
31 Won, J. S. & Singh, I. Sphingolipid signaling and redox regulation. Free Radic Biol Med 40, 1875-1888 (2006).
32 Terce, F., Brun, H. & Vance, D. E. Requirement of phosphatidylcholine for normal progression through the cell cycle in C3H/10T1/2 fibroblasts. J Lipid Res 35, 2130-2142 (1994).
33 Kim, H. Y., Huang, B. X. & Spector, A. A. Phosphatidylserine in the brain: metabolism and function. Prog Lipid Res 56, 1-18 (2014).
34 Hussain, G. et al. Role of cholesterol and sphingolipids in brain development and neurological diseases. Lipids Health Dis 18, 26 (2019).
35 Tanguy, E., Wang, Q., Moine, H. & Vitale, N. Phosphatidic Acid: From Pleiotropic Functions to Neuronal Pathology. Front Cell Neurosci 13, 2 (2019).
36 Verdin, E. & Ott, M. 50 years of protein acetylation: from gene regulation to epigenetics, metabolism and beyond. Nat Rev Mol Cell Biol 16, 258-264 (2015).
37 Harachi, M., Masui, K., Cavenee, W. K., Mischel, P. S. & Shibata, N. Protein Acetylation at the Interface of Genetics, Epigenetics and Environment in Cancer. Metabolites 11 (2021).
38 Blumenberg, L. et al. BlackSheep: A Bioconductor and Bioconda Package for Differential Extreme Value Analysis. J Proteome Res 20, 3767-3773 (2021).
39 Guarente, L. The logic linking protein acetylation and metabolism. Cell Metab 14, 151-153 (2011).
40 Yuan, H. et al. MYST protein acetyltransferase activity requires active site lysine autoacetylation. EMBO J 31, 58-70 (2012).
41 McCullough, C. E., Song, S., Shin, M. H., Johnson, F. B. & Marmorstein, R. Structural and Functional Role of Acetyltransferase hMOF K274 Autoacetylation. J Biol Chem 291, 18190-18198 (2016).
42 Karanam, B., Jiang, L., Wang, L., Kelleher, N. L. & Cole, P. A. Kinetic and mass spectrometric analysis of p300 histone acetyltransferase domain autoacetylation. J Biol Chem 281, 40292-40301 (2006).
43 Black, J. C., Mosley, A., Kitada, T., Washburn, M. & Carey, M. The SIRT2 deacetylase regulates autoacetylation of p300. Mol Cell 32, 449-455 (2008).
44 Santos-Rosa, H., Valls, E., Kouzarides, T. & Martinez-Balbas, M. Mechanisms of P/CAF auto-acetylation. Nucleic Acids Res 31, 4285-4292 (2003).
45 Shandilya, J. et al. Acetylated NPM1 localizes in the nucleoplasm and regulates transcriptional activation of genes implicated in oral cancer manifestation. Mol Cell Biol 29, 5115-5127 (2009).
46 Kaypee, S. et al. Mutant and Wild-Type Tumor Suppressor p53 Induces p300 Autoacetylation. iScience 4, 260-272 (2018).
47 Sun, Y., Xu, Y., Roy, K. & Price, B. D. DNA damage-induced acetylation of lysine 3016 of ATM activates ATM kinase activity. Mol Cell Biol 27, 8502-8509 (2007).
48 Matsuoka, S. et al. ATM and ATR substrate analysis reveals extensive protein networks responsive to DNA damage. Science 316, 1160-1166 (2007).
49 Beli, P. et al. Proteomic investigations reveal a role for RNA processing factor THRAP3 in the DNA damage response. Mol Cell 46, 212-225 (2012).
50 Bensimon, A. et al. ATM-dependent and -independent dynamics of the nuclear phosphoproteome after DNA damage. Sci Signal 3, rs3 (2010).
51 Stokes, M. P. et al. Profiling of UV-induced ATM/ATR signaling pathways. Proc Natl Acad Sci USA 104, 19855-19860 (2007).
52 Elia, A. E. et al. Quantitative Proteomic Atlas of Ubiquitination and Acetylation in the DNA Damage Response. Mol Cell 59, 867-881 (2015).
53 Zhang, Y. & Hunter, T. Roles of Chk1 in cell biology and cancer therapy. Int J Cancer 134, 1013-1023 (2014).
54 Boja, E. S. & Rodriguez, H. Mass spectrometry-based targeted quantitative proteomics: achieving sensitive and reproducible detection of proteins. Proteomics 12, 1093-1110 (2012).
55 Ma, W. et al. DreamAI: algorithm for the imputation of proteomics data. bioRxiv, 2020.2007.2021.214205 (2021).
56 Frattini, V. et al. A metabolic function of FGFR3-TACC3 gene fusions in cancer. Nature 553, 222-227 (2018).
57 Wiredja, D. D., Koyuturk, M. & Chance, M. R. The KSEA App: a web-based tool for kinase activity inference from quantitative phosphoproteomics. Bioinformatics 33, 3489-3491 (2017).
58 Hernandez-Armenta, C., Ochoa, D., Goncalves, E., Saez-Rodriguez, J. & Beltrao, P. Benchmarking substrate-based kinase activity inference using phosphoproteomic data. Bioinformatics 33, 1845-1851 (2017).
59 Kuleshov, M. V. et al. KEA3: improved kinase enrichment analysis via data integration. Nucleic Acids Res 49, W304-W316 (2021).
60 Ochoa, D. et al. An atlas of human kinase regulation. Mol Syst Biol 12, 888 (2016).
61 Yilmaz, S. et al. Robust inference of kinase activity using functional networks. Nat Commun 12 (2021).
62 Bezy, O. et al. PKCdelta regulates hepatic insulin sensitivity and hepatosteatosis in mice and humans. J Clin Invest 121, 2504-2517 (2011).
63 Mayr, M. et al. Loss of PKC-delta alters cardiac metabolism. Am J Physiol Heart Circ Physiol 287, H937-945 (2004).
64 Mayr, M. et al. Proteomic and metabolomic analysis of vascular smooth muscle cells: role of PKCdelta. Circ Res 94, e87-96 (2004).
65 Magaway, C., Kim, E. & Jacinto, E. Targeting mTOR and Metabolism in Cancer: Lessons and Innovations. Cells 8 (2019).
66 Zhan, J., Chitta, R. K., Harwood, F. C. & Grosveld, G. C. Phosphorylation of TSC2 by PKC-delta reveals a novel signaling pathway that couples protein synthesis to mTORC1 activity. Mol Cell Biochem 456, 123-134 (2019).
67 Li, W. et al. Protein kinase C-delta is an important signaling molecule in insulin-like growth factor I receptor-mediated cell transformation. Mol Cell Biol 18, 5888-5898 (1998).
68 Kwon, J. et al. Insulin receptor substrate-2 mediated insulin-like growth factor-I receptor overexpression in pancreatic adenocarcinoma through protein kinase Cdelta. Cancer Res 69, 1350-1357 (2009).
69 Gibbs, P. E., Miralem, T., Lerner-Marmarosh, N., Tudor, C. & Maines, M. D. Formation of ternary complex of human biliverdin reductase-protein kinase Cdelta-ERK2 protein is essential for ERK2-mediated activation of Elk1 protein, nuclear factor-kappaB, and inducible nitric-oxidase synthase (iNOS). J Biol Chem 287, 1066-1079 (2012).
70 Kim, H., Na, Y. R., Kim, S. Y. & Yang, E. G. Protein Kinase C Isoforms Differentially Regulate Hypoxia-Inducible Factor-1alpha Accumulation in Cancer Cells. J Cell Biochem 117, 647-658 (2016).
71 Lee, P. C. et al. Targeting PKCdelta as a Therapeutic Strategy against Heterogeneous Mechanisms of EGFR Inhibitor Resistance in EGFR-Mutant Lung Cancer. Cancer Cell 34, 954-969 e954 (2018).
72 Cheng, J. et al. The Caspase-3/PKCdelta/Akt/VEGF-A Signaling Pathway Mediates Tumor Repopulation during Radiotherapy. Clin Cancer Res 25, 3732-3743 (2019).
73 Kim, M. J. et al. Importance of PKCdelta signaling in fractionated-radiation-induced expansion of glioma-initiating cells and resistance to cancer treatment. J Cell Sci 124, 3084-3094 (2011).
74 Chen, C. H. et al. PKCdelta-mediated SGLT1 upregulation confers the acquired resistance of NSCLC to EGFR TKIs. Oncogene 40, 4796-4808 (2021).
75 Steinberg, S. F. Distinctive activation mechanisms and functions for protein kinase C delta. Biochem J 384, 449-459 (2004).
76 Takashima, A. et al. Protein kinase Cdelta is a therapeutic target in malignant melanoma with NRAS mutation. ACS Chem Biol 9, 1003-1014 (2014).
77 Blackford, A. N. & Jackson, S. P. ATM, ATR, and DNA-PK: The Trinity at the Heart of the DNA Damage Response. Mol Cell 66, 801-817 (2017).
78 Yue, X., Bai, C., Xie, D., Ma, T. & Zhou, P. K. DNA-PKcs: A Multi-Faceted Player in DNA Damage Response. Front Genet 11, 607428 (2020).
79 Buisson, R., Boisvert, J. L., Benes, C. H. & Zou, L. Distinct but Concerted Roles of ATR, DNA-PK, and Chk1 in Countering Replication Stress during S Phase. Mol Cell 59, 1011-1024 (2015).
80 Liu, S. et al. Distinct roles for DNA-PK, ATM and ATR in RPA phosphorylation and checkpoint activation in response to replication stress. Nucleic Acids Res 40, 10780-10794 (2012).
81 Majd, N. K. et al. The promise of DNA damage response inhibitors for the treatment of glioblastoma. Neurooncol Adv 3, vdab015 (2021).
82 Stupp, R. et al. Radiotherapy plus concomitant and adjuvant temozolomide for glioblastoma. N Engl J Med 352, 987-996 (2005).
83 Chen, B. P. et al. Cell cycle dependence of DNA-dependent protein kinase phosphorylation in response to DNA double strand breaks. J Biol Chem 280, 14709-14715 (2005).
84 Chan, D. W. et al. Autophosphorylation of the DNA-dependent protein kinase catalytic subunit is required for rejoining of DNA double-strand breaks. Genes Dev 16, 2333-2338 (2002).
85 Ziv, Y. et al. Chromatin relaxation in response to DNA double-strand breaks is modulated by a novel ATM- and KAP-1 dependent pathway. Nat Cell Biol 8, 870-876 (2006).
86 Paull, T. T. & Lee, J. H. The Mre11/Rad50/Nbs1 complex and its role as a DNA double-strand break sensor for ATM. Cell Cycle 4, 737-740 (2005).
87 Petralia, F. et al. Integrated Proteogenomic Characterization across Major Histological Types of Pediatric Brain Cancer. Cell 183, 1962-1985 e1931 (2020).
88 Satpathy, S. et al. A proteogenomic portrait of lung squamous cell carcinoma. Cell 184, 4348-4371 e4340 (2021).
89 Behling, F. & Schittenhelm, J. Oncogenic BRAF Alterations and Their Role in Brain Tumors. Cancers (Basel) 11 (2019).
90 Ryall, S., Tabori, U. & Hawkins, C. Pediatric low-grade glioma in the era of molecular diagnostics. Acta Neuropathol Commun 8, 30 (2020).
91 Lassaletta, A. et al. Therapeutic and Prognostic Implications of BRAF V600E in Pediatric Low-Grade Gliomas. J Clin Oncol 35, 2934-2941 (2017).
92 Holloway, R. W. & Marignani, P. A. Targeting mTOR and Glycolysis in HER2-Positive Breast Cancer. Cancers (Basel) 13 (2021).
93 Wilkerson, M. D. et al. Lung squamous cell carcinoma mRNA expression subtypes are reproducible, clinically important, and correspond to normal cell types. Clin Cancer Res 16, 4864-4875 (2010).
94 Corsello, S. M. et al. Discovering the anti-cancer potential of non-oncology drugs by systematic viability profiling. Nat Cancer 1, 235-248 (2020).
95 Akiyoshi, T., Matzno, S., Sakai, M., Okamura, N. & Matsuyama, K. The potential of vitamin K3 as an anticancer agent against breast cancer that acts via the mitochondria-related apoptotic pathway. Cancer Chemother Pharmacol 65, 143-150 (2009).
96 Turnbull, A. K. et al. Unlocking the transcriptomic potential of formalin-fixed paraffin embedded clinical tissues: comparison of gene expression profiling approaches. BMC Bioinformatics 21, 30 (2020).
97 Stewart, J. P. et al. Standardising RNA profiling based biomarker application in cancer—The need for robust control of technical variables. Biochim Biophys Acta Rev Cancer 1868, 258-272 (2017).
98 Li, J., Fu, C., Speed, T. P., Wang, W. & Symmans, W. F. Accurate RNA Sequencing From Formalin-Fixed Cancer Tissue To Represent High-Quality Transcriptome From Frozen Tissue. JCO Precis Oncol 2018 (2018).
99 Friedman, J., Hastie, T. & Tibshirani, R. Regularization Paths for Generalized Linear Models via Coordinate Descent. J Stat Softw 33, 1-22 (2010).
100 Simon, N., Friedman, J. & Hastie, T. A Blockwise Descent Algorithm for Group-penalized Multiresponse and Multinomial Regression. arXiv:1311.6529 (2013).
101 Tibshirani, R. et al. Strong rules for discarding predictors in lasso-type problems. J R Stat Soc Series B Stat Methodol 74, 245-266 (2012).
102 Wright, G. W. et al. A Probabilistic Classification Tool for Genetic Subtypes of Diffuse Large B Cell Lymphoma with Therapeutic Implications. Cancer Cell 37, 551-568 e514 (2020).
103 Roos, W. P. & Krumm, A. The multifaceted influence of histone deacetylases on DNA damage signalling and DNA repair. Nucleic Acids Res 44, 10017-10030 (2016).
104 Gillette, M. A. et al. Proteogenomic Characterization Reveals Therapeutic Vulnerabilities in Lung Adenocarcinoma. Cell 182, 200-225 e235 (2020).
105 Carro, M. S. et al. The transcriptional network for mesenchymal transformation of brain tumours. Nature 463, 318-325 (2010).
106 Lambert, M., Jambon, S., Depauw, S. & David-Cordonnier, M. H. Targeting Transcription Factors for Cancer Treatment. Molecules 23 (2018).
107 Roskoski, R., Jr. Properties of FDA-approved small molecule protein kinase inhibitors: A 2021 update. Pharmacol Res 165, 105463 (2021).
108 Cleary, J. M., Aguirre, A. J., Shapiro, G. I. & D'Andrea, A. D. Biomarker-Guided Development of DNA Repair Inhibitors. Mol Cell 78, 1070-1085 (2020).

### Methods

### Patient Datasets and Profiling Platforms

Glioblastoma (GBM) IDH wild type, pediatric glioma (PG), breast cancer (BRCA), and lung squamous cell carcinoma (LSCC) data were obtained from the Clinical Proteomic Tumor Analysis Consortium (CPTAC). Ninety-two GBM IDH wild type were included in the analysis¹⁴. PG included 105 high and low-grade glioma⁸⁷; BRCA included 119 tumors⁹ and LSCC consisted of 108 tumors⁸⁸. Analyses were performed using RNA-Seq, proteomics and phospho-proteomics data for IDH wild type GBM, PG, BRCA and LSCC; DNA methylation, copy number, acetylomics, lipidomics, and metabolomics data were used for IDH wild type GBM. Additional DNA methylation data were obtained from TCGA for GBM (140 tumors profiled by Illumina Infinium Human Methylation 450 K and 287 tumors profiled by Infinium HumanMethylation27 BeadChip). Additional RNA-seq data were obtained from TCGA for BRCA (1,095 tumors)^{109,110} and lung squamous cell carcinoma (LUSC, 502 tumors)¹¹¹; microarray data were obtained from BRCA METABRIC (1,904 tumors)¹¹²¹¹³. Clinical and survival data were also available.

### Data Processing

RNA-Sequencing (CPTAC-GBM, CPTAC-PG, CPTAC-BRCA, CPTAC-LSCC): RNA-Seq data were downloaded as fpkm. Non-protein-coding and low-expressed genes were removed from the analysis. Expression data were quantile and log₂ normalized.

DNA methylation (CPTAC-GBM): DNA methylation data from Illumina infinium methylationEPIC beadchip array were downloaded as beta values, pre-processed with functional normalization, quality check, common SNP filtering, and probe annotation.

Copy-number (CPTAC-GBM): Thresholds for CN variations (CNV) calls were assessed using GISTIC scores (−2 homozygous deletion, −1 heterozygous deletion, 0 no change, 1 gain, 2 amplification). Non-protein-coding genes were removed from the analysis. To select CNV calls that impact gene expression in cis, functional copy number variation (fCNV) analysis was performed as described¹³.

Global proteome and phospho-proteome (CPTAC-GBM, CPTAC-PG, CPTAC-BRCA, CPTAC-LSCC): Missing values in global proteome and phospho-proteome data were imputed with the DreamAI algorithm⁵⁵ and log₂-transformed. Final matrices were quantile normalized.

Lipidome and Metabolome (CPTAC-GBM): Lipidome and metabolome data were downloaded as7 log₂-transformed and median normalized. Lipids and metabolites with missing values in more than 5 and 10 tumors, respectively were excluded from the analysis. For the remaining lipids and metabolites, the average metabolite abundance was used to impute abundance in tumors with missing values. The final matrices were quantile normalized.

Acetylome (CPTAC-GBM): Acetylome data were imputed with DreamAI algorithm and log₂-transformed.

mRNA expression (METABRIC-BRCA): RNA expression data profiled by microarray using Illumina HT-12 v 3 platform were downloaded as median normalized.

RNA-Sequencing (TCGA-LUSC and TCGA-BRCA): RNA-seq data were downloaded using the TCGAbiolinks R/Bioconductor package. We applied GC content correction to raw data for the within-normalization step and upper quantile for the between phase.

DNA-methylation (TCGA-GBM): DNA methylation data profiled by Illumina Infinium Human Methylation 450 K platform and Infinium HumanMethylation27 BeadChip were downloaded using TCGAbiolinks package available on R Bioconductor. Data were normalized using functional normalization as implemented in minfi¹¹⁴ and probes targeting X and Y chromosomes or not associated with gene promoters¹¹⁵ were removed.

### Functional Classification of the CPTAC IDH Wild Type GBM Cohort

To assign functional GBM subtype membership to the 92 CPTAC-GBM IDH wild type tumors, we developed a multi-step computational approach. First, we used Agilent expression profiles of 304 IDH wild type GBM from TCGA that we had previously classified¹³ as the training set of a k-Nearest Neighbors (k-NN classifier, k=3). The classifier feature set included expression of the 100 highest scoring genes in the ranked list of each transcriptome-based subtype. To account for possible differences in gene expression data between TCGA and CPTAC, we generated the ranked lists of genes with differential expression in each GBM subtype compared to the others using the CPTAC transcriptomic data and the Mann Whitney Wilcoxon (MWW) test. We defined as subtype-specific gene expression signatures the 50 highest scoring genes in the ranked lists. Finally, for each tumor, we derived the intensity of each functional state as the average expression of the genes included in each subtype specific signature. For each tumor, the simplicity score was derived as the difference between the two highest state intensities. We retained only tumors with simplicity score higher than 0.6. This classification included 17 GPM, 6 MTC, 16 NEU and 13 PPR for a total of 52 tumors, defined core samples.

To assign the membership to 40 unclassified tumors, we integrated fCNV and gene expression profiles using the similarity network fusion (SNF) method for the 89 tumors for which both data were available from CPTAC. The set of features of the classifier included fCNV gains/losses we previously identified as subtype-specific from TCGA and subtype specific gene expression signatures from CPTAC core tumors. The two data types were aggregated by SNFtool R package to generate a fused tumor network and a fused tumor similarity matrix using the following parameters: K=20, alpha=0.5, and t=20. Spectral clustering was performed on the fused tumor similarity matrix using the function built into the SNF package. The fused tumor similarity matrix generated from SNF approach was used to derive a distance matrix (1-similarity). The distance matrix (1-W) was then utilized to establish membership of 38 unclassified GBM that had CN and RNA-Seq available, according to closeness to the previously classified core tumors using k-NN (k=3). Five tumors with conditional probability of subtype memberships <0.6 remained unclassified. Unequivocally classified tumors includes 22 GPM, 12 MTC, 23 NEU and 28 PPR for a total of 85 tumors.

### Cross-Classification Analysis

We independently classify both TCGA and CPTAC GBM samples profiled by methylation according the MolecularNeuroPathology (MNP) DNA Methylation Classification²⁰. We included probes from CPTAC samples with <20% of missing values across all samples. The remaining missing values were imputed using the mean of the corresponding probe value. The processed microarray beta values and classification of the MNP cohort (v11b2) were downloaded from the GEO dataset GSE90496 (MNP reference set) and supplemental tables of the original publication, consisting of 347 GBM classified by MNP.

We utilized k-NN using as training set the MNP samples with MNP assignment and as test sets the TCGA or CPTAC dataset. The top 10,000 variable probes out of the shared probes across MNP and TCGA- or CPTAC-GBM samples were selected to construct the shared DNA methylome. We extracted the top 30 principal components by PCA and assigned MNP classification to TCGA or CPTAC samples using the k-NN classifier (k=9)¹⁴. While an official MNP DNA methylation classifier exists online (www.molecularneuropathology.org/mnp), we were not able to access the classifier since our registration was not approved by the site at the time of writing.

We assessed the relationship between pathway-based classification and transcriptional subtyping both in TCGA and CPTAC datasets. We analyzed 304 TCGA-GBM for which an unequivocal pathway-based subtype assignment had been obtained¹³. GBM subtype assignments according to the transcriptional signatures proposed by the TCGA were obtained by applying ssGSEA as described in the original report¹⁹. Both transcriptional and multiomics-based subtyping of CPTAC-GBM tumors were from supplemental tables of the original publication¹⁴.

### Multi-Omics Characterization of the Four Functional Subtypes in GBM

We used the MWW test to generate ranked lists of genes/proteins differentially expressed/abundant in each of the subtypes compared to the others. For each subtype the final gene/protein signature included the first 150 highest scoring genes/proteins in the ranked list.

These gene/protein signatures were used to calculate the enrichment of each functional GBM subtype (normalized enrichment score of subtype activity, NES) for each tumor using single-sample MWW-GST (ssMWW-GST).

To extract the distinct biological functions of each cluster, we used MSigDB c5.bp, c5.mf, c5.cc, Hallmark and KEGG collections of gene sets, retaining only pathways composed of at least 15 genes, resulting in 5,032 gene sets. We removed gene sets that included more than 250 genes (650 gene pathways), thus avoiding the preferential selection of very large pathways, and applying the enrichment set cover algorithm to retain not-redundant pathways¹¹⁶. Finally, we performed two-sided MWW-GST to identify the most active pathways in each subtype using both gene and protein ranked lists [logit(NES)>0.58, FDR<0.005].

To identify novel metabolic features/activities in the GPM and MTC subtypes, we first compared the protein abundance of key enzymes catalyzing glycolysis, pyruvate transport, TCA cycle or assembling the respiratory chain mitochondrial complexes, and metabolite abundance of intermediates involved in key glycolytic and mitochondrial pathways. Using the ranked lists of proteins and metabolites differentially abundant in GPM versus MTC subtypes, we applied MWW-GST to generate the enrichment of glycolytic and mitochondrial enzymes as protein sets, and metabolic intermediates as metabolite set, in each subtype [glycolytic enzymes: logit(NES)=1.27, p=0.017; mitochondrial enzymes: logit(NES)=−1.19, p=5.93e-13; glycolytic metabolic intermediates: logit(NES)=1.76, p=0.0007; mitochondrial metabolic intermediates: logit(NES)=−1.65, p=0.018]. The interactome network of metabolites and metabolic proteins was constructed using knowledge-based interactions by the Ingenuity pathway analysis tool (IPA)¹¹⁷.

We used the MWW test to generate ranked lists of lipid molecules with differential abundance in each of the subtypes compared to the others. For each subtype, the final lipid molecule signature included lipids with an MWW score >0.5 in the ranked list. To explore the enrichment of different lipid subclasses in the four subtypes, we used the series of lipid molecules profiled to create a lipid ontology of the distinct lipid subclasses (acylcarnitine, ceramide, cholesteryl ester, diacylglycerol, hexosylceramide, phosphatidic acid, phosphatidylcholine, phosphatidylethanolamine, phosphatidylglycerol, phosphatidylinositol, phosphatidylserine, sphingomyelin, triacylglycerol). We used subtype-specific lipid molecule signatures to derive enrichment of the lipid subclasses, cellular components and lipid functions in each subtype using Fisher exact test (log odd-ratio>0 and p<0.05) and the lipid ontology database, LION, which provides information on predominant sub-cellular localization (cellular components) and functions of more than 50,000 lipid species²⁵.

### Proteogenomic Integrative Analysis of GBM

To identify genes whose gain/loss corresponded to increase/decrease subtype-specific gene expression and increase/decrease protein abundance (fCNVprot), we integrated gene expression, fCNV and protein abundance. Gene alterations that exhibited fCNV change in at least 2 tumors were retained in the analysis if the following criteria were met: i) higher/lower abundance of the corresponding protein in tumors with alteration when compared to wild type tumors (|log 2(FC)|>0.15, p<0.10, two-sided MWW test); ii) higher/lower abundance of the corresponding protein in the GBM subtype when compared to the others (|log₂(FC)|>0.15, p<0.10, two-sided MWW test); iii) higher subtype-specific transcriptomic activity of the tumor harboring the fCNV compared with wild type tumors (effect size >0.15, p<0.10; two-sided MWW test). The resulted subtype-associated fCNV^{prot} gains and losses were used to derive their contribution to activation/de-activation of the biological pathways using the Fisher exact test (p<0.05).

Univariate logistic regression analysis was performed to model fCNV amplification/deletion status in GBM driver genes as function of subtype transcriptomic activity (model 1) or subtype assignment as function of protein abundance of GBM driver genes (model 2) in the CPTAC-GBM cohort (n=84 tumors). In model 1, tumors were segregated according to altered or wild-type fCNV status while subtype activity (NES) was used as continuous predictor variable. In model 2, tumors were segregated according to group assignment (GPM, MTC, NEU or PPR versus all others) while protein abundance was used as continuous predictor variable. For fCNV deletions, we multiplied subtype activity (NES) by −1 for visualization purpose. For FGFR3-TACC3 fusion, tumors from the GBM FFPE RNA-Seq cohort of 178 GBM were classified (GPM, MTC, NEU or PPR) and FGFR3-TACC3 fusion status (present, 12 tumors or absent) was used as predictor variable.

### Analysis of Acetylation of Metabolic and Nuclear Proteins in GPM, MTC and PPR Subtypes

We used Reactome Metabolism gene set containing 2,212 genes to define proteins involved in metabolism¹¹⁸. Unsupervised clustering was performed on metabolic proteins with differential expression between GPM and MTC subtypes (MWW test, p<0.05, log²(fold-change)>0.3). To identify sites whose acetylation was not explained by the abundance of the corresponding protein, we calculated the normalized acetylation values as the residuals of each acetylated site (εₛᵢₜₑ) derived from the linear regression model Acₛᵢₜₑ=β₀+β₁·Prₛᵢₜₑ+εₛᵢₜₑ, where Acₛᵢₜₑ is the acetylation abundance of a given site, and Prₛᵢₜₑ is the protein abundance of the corresponding protein. We applied BlackSheep's differential extreme value analysis module to define the list of outlier acetylated metabolic proteins in GPM and MTC subtypes (p<0.05). For the i-th protein in the j-th tumor, outlier fraction was defined as positive values (from 0 to 1) for the fraction of hyper acetylated sites of the i-th protein, or as negative values (from 0 to −1) for the fraction of hypo acetylated sites. Low outlier fractions (values between ±0.1) were not shown in the heat map. Enrichment of biological pathways by significant outlier acetylated metabolic proteins identified in GPM and MTC subtypes has been performed using Fisher exact test (p<0.0005).

Nuclear proteins were determined by the COMPARTMENTS database¹⁹ with the nucleus score=5 and the subcellular localization of the protein was further assessed using the Ingenuity pathway analysis tool. Acetylation was used to define the acetylated sites (n=320) with highest variability across the dataset by inter quartile range (IQR). Association between clusters identified by unsupervised clustering using the most variable acetylated sites and GBM functional subtypes was assessed by the χ2 test. Differentially abundant acetylated sites of nuclear proteins in high versus low nuclear protein acetylation PPR subgroups were defined using MWW test [p<0.001, log₂(FC)>0.3]. To identify only sites whose acetylation is not explained by protein abundance, global protein and acetyl site abundance was compared between high and low nuclear protein acetylation PPR subgroups using Wilcoxon test. Acetylation or protein changes were considered significantly different if they had p<0.05 and log₂(FC)>0.5. Then, we selected only sites with significant hyperacetylation, in the absence of significant protein change. Pathway and GO term overrepresentation testing were performed using gProfiler tool (FDR<0.05).

### Generation of Replication Stress/DNA Damage Response Phospho-Proteomic Signature

To test the status of the DNA damage repair induced-phosphorylation events in the functional GBM subtypes, we assembled five different studies of mass-spectrometry-based phosho-proteomics⁴⁸⁻⁵². We defined a curated series of sites whose phosphorylation was increased after induction of DNA replication stress (with ATR inhibition or hydroxyurea treatment, RS) or DNA damage (with irradiation). Differential phospho-abundance of DDR/RS induced-sites was performed using Wilcoxon comparing PPR subgroup versus all the others (p<0.05). RS/DDR phospho-signatures were used to calculate the NES for each tumor as DDR/RS phospho-based scores using ssMWW-GST. We tested the enrichment of GPM, MTC, NEU and PPR tumors in the highest or lowest distribution of the DDR/RS score [abs(logit(NES))>0] using Fisher exact test. In addition, for each tumor and subtype, we derived the difference between transcriptome-based and global proteome based-subtype activity and tested the association with DDR/RS score using Spearman's correlation.

### Functional Classification of PG, BRCA and LSCC

To assign functional subtype membership to the tumors included in the CPTAC-PG, -BRCA and -LSCC cohorts, we developed a multi-step computational approach. First, we used the RNA-seq expression profiles of 105 PG, 119 BRCA and 108 LSCC to compute single-sample enrichment of the 50 highest scoring genes that we had previously identified as subtype-specific from TCGA-GBM using MWW-GST. Each tumor was classified according to the subtype with the highest NES [logit(NES)>0.3 & FDR<0.05]. We then used protein abundance data to compute the single sample enrichment of the 50 highest scoring proteins in the ranked list of each CPTAC GBM subtype using MWW-GST. Each tumor was classified according to the subtype with the highest NES [logit(NES)>0.3 & FDR<0.05]. We defined anchors tumors that obtained the same subtype membership from transcriptomic and proteomic data (51, 23, 64 tumors for PG, BRCA and LSCC, respectively). We then used anchor tumors to generate the ranked lists of genes and proteins differentially expressed in each subtype compared with the others using the MWW test and obtained new subtype-specific gene and protein signatures, including the first 50 highest scoring genes and proteins in the ranked list. Unclassified tumors with gene expression and proteomics data available, were classified by integrating gene and protein signatures from the previous step using SNF. The two data types were aggregated by SNFtool R package to generate a fused tumor network and a fused tumor similarity matrix using the following parameters: k=20, alpha=0.5, and t=20. The fused tumor similarity matrix generated by SNF was used to obtain a distance matrix (1-similarity) and establish the membership of the unclassified samples (54 PG, 96 BRCA and 44 LSCC) according to their closeness to the previously classified anchor tumors using the k-NN classifier (k=3). Tumors with conditional probability of subtype memberships <0.6 remained unclassified. The final classification includes 48 GPM, 7 MTC, 27 NEU and 22 PPR, and 1 unclassified for PG; 50 GPM, 23 MTC, 5 NEU and 40 PPR for BRCA; 51 GPM, 9 MTC, 0 NEU and 46 PPR, and 2 unclassified for LSCC samples. We used the expression profiles of 1,095 tumors from TCGA-BRCA, 1,904 tumors from METABRIC-BRCA and 502 tumors from TCGA LUSC to compute single-sample enrichment of the 50 highest scoring genes we previously identified as subtype-specific from TCGA using MWW-GST. Each tumor was classified according to the subtype with the highest NES [logit(NES)>0.58 & FDR<0.05].

### Analysis of DepMap

We used the BRCA and LUSC cell lines cohorts from DepMap for which both RNA-Seq expression and menadione survival ratio from PRISM Repurposing Primary Screen were available (n=26 for BRCA and n=71 for LUSC)⁹⁴. For each cell line, we used the transcriptomic profile to calculated the NES of the four functional GBM subtypes by MWW-GST using gene signatures obtained from the analysis of TCGA samples. Each cell line was classified according the highest subtype activity (NES). We assessed the difference in survival ratio between the mitochondrial cell lines and the others using two-sided Student t-test, unequal variance.

### Characterization of the Four Functional Subtypes in CPTAC-PG CPTAC-BRCA, CPTAC-LSCC, TCGA-BRCA, METABRIC-BRCA, TCGA-LUSC

For CPTAC-PG we used the MWW test to derive ranked lists of genes/proteins/phospho-proteins differentially expressed/abundant in each of the subtypes compared to the others. For each subtype the final gene/protein/phospho-proteins signature included the first 150 highest scoring genes/proteins/phospho-proteins in the ranked list. Pathways enriched by each gene/protein/phospho-protein subclusters identified by unsupervised clustering were defined using Fisher exact test (p<0.05). Association between functional subtype based-classification and tumor grade, BRAF status (PG) or CPTAC-NMF derived subtypes (BRCA and LSCC) was assessed by the χ2 test. Difference in survival among functional subtypes in TCGA-BRCA, TCGA-LUSC, METABRIC-BRCA was assessed by log-rank test.

### Semi-Supervised Support Vector Machine (SVM) for Substrate Phosphosite-Informed Inference Network of Kinases (SPHINKS)

To identify putative kinase substrate targets, we implemented a machine learning method, SPHINKS (substrate phosphosite-informed inference network of kinases) which starts from observed data to generalize unseen data using semi-supervised approaches already applied in the reconstruction of gene regulatory networks^{120,121}. The goal was to create an unbiased kinome network leveraging kinases abundance from proteomics, substrate abundance from phoshoproteomics, and validated interactions available from PhosphoSitePlus¹²². The reconstruction of the network was a binary model classification in which the statistical classifier was trained to recognize relationships between abundance profiles of kinase-phosphosite pairs. Positive training set was the set of known substrates of a specific kinase. This represented the typical setting where a learner has access only to positive examples and unlabeled data (containing both positive and negative examples), whereby even for the best studied kinases there are many potential kinase-substrate relationships to be considered or discovered. The problem, known in machine learning as Positive Unlabeled (PU)¹²¹, had the additional complication of the highly imbalanced positive set and unlabeled samples, consisting of the space of potential novel interactions. We designed an approach based on the combination of easyensamble approach¹²³ and the bootstrap aggregating (bagging) machine learning ensemble meta-algorithm¹²⁴ that combines several Support Vector Machine classifiers trained on different instances of the negative set (FIG. 12a). First, an SVM classifier was trained on the experimentally validated kinase-substrate interactions as positive training set and a subset of randomly selected unknown interactions as negative set. Each training example of the SVM represents a kinase-phospho-substrate interaction and contains the juxtaposition of the kinase's protein abundance and the substrate's phospho-protein abundance on a set of corresponding cases. This forms a training matrix having the examples along the rows. Next, by building a new matrix for all the possible kinase-substrate pairs in the dataset, we obtained a score (a value between 0 and 1), representing the probability for each phospho-site to be a kinase substrate according to the SVM classifier. To improve the stability and accuracy of the prediction of the score for each kinase-substrate pair, we applied bagging keeping fixed the positive set with multiple random samples of the negative set. The first two steps were repeated 100 times, to derive the SPHINKS scores as the average result of the SVM outputs from all iterations.

To create a set of predicted substrates (SOPS) for each kinase, i.e., the list of predicted kinase substrate interactions, we selected a threshold for the average score as the score for which at least 50% of the known interactions were kept. As further filter criterion, we retained the interactions between kinase and substrate phosphosite whose Spearman's correlation was positive. Finally, we removed kinases with less than 10 interactions. For GBM samples, we obtained a kinase-substrate interactome comprising 13,866 interactions (median SOPS size: 50) including 154 kinases and 3,186 phospho-sites. For PG samples, the network is composed of 669 interactions (median SOPS size: 4) including 76 kinases and 210 phospho-sites. For BRCA samples, 13,943 interactions (median SOPS size: 50) including 198 kinases and 1,899 phospho-sites. For LSCC samples, 1,985 interactions (median SOPS size: 6) including 103 kinases and 669 phospho-sites.

### Identification of GBM Subtype-Specific Master Kinases (MKs)

To identify MKs responsible for the phospho-site signatures activated in the four functional GBM subtypes, we modified a method that we had previously described for the calculation of master regulators activity⁵⁶. In brief, the activity of a master kinase MK is defined as the index that quantify the activation of the substrate program of that specific MK in each sample Xᵢ (i=1, . . . , 85). First, we bin all substrates into 25 bins according to their average abundance across all samples. For each kinase MK, let us define {s₁, . . . , sₖ} the substrates in the SOPS of MK. Then, we randomly extract a set of n=100 control substrates for each sₖ from the corresponding bin {c₁, . . . , c_{100K}}. In this way, the control substrate-set has distribution of abundance levels comparable to that of SOPS, while being 100-fold larger. The activity of the kinase MK in the sample xᵢ is computes as:

$$Act ( {X}_{i} , MK ) = \frac{{\sum }_{k = 1}^{K} {ω}_{{s}_{k}} \cdot {t}_{{s}_{k}}^{i}}{{\sum }_{k = 1}^{K} {ω}_{{S}_{k}}} - \frac{{\sum }_{j = 1}^{100 K} {ω}_{{c}_{j}} \cdot {t}_{{c}_{j}}^{i}}{{\sum }_{j = 1}^{100 K} {ω}_{{c}_{j}}} ,$$

where wₛₖ is the SPHINKS score of the k-th substrate of the kinase MK; w_{cj} is the SPHINKS score of the j-th control substrate; tₛ_{ₖ}ⁱ and t_{c}_{ⱼ}ⁱ are the phospho-site abundance of the substrate sₖ or cⱼ in the i-th sample, respectively.

If Act(Xᵢ, MK)>0, the j-th master kinase is activated in the i-th sample, if Act(Xᵢ, MK)<0, the j-th master kinase is inversely activated, if Act(Xᵢ, MK)≈0, it is deactivated. We used the MWW test to select master kinases that showed a significant difference in activity in one subtype compared with the others (effect size >0.3 and p<0.01, MWW test). Finally, significantly active master kinases in each GBM subtype were mapped on a kinome tree using KinMap online tool¹²⁵.

### Benchmarking of Kinase-Phospho-Substrate Interaction Predictions and Kinase Activity by SPHINKS

Impact of missing values and imputation algorithm on the performance of SPHINKS

In all our analysis, proteins/phospho-sites with missing values in less than 50% of samples in the dataset were imputed using the DreamAI algorithm. In order to establish how the prediction of the true positive kinase-phospho-substrate interactions degrades as the level of imputation increases, we performed a set of simulations in a controlled setting where we can have a gold standard for the evaluation of the stability of the SPHINKS predictions. First, we selected from the un-imputed phospho-proteomic data of the CPTAC-GBM cohort only those sites that have no missing values (n=7,302 phospho-sites). We applied SPHINKS to this matrix to generate a network of kinase phospho-site interactions unaffected by missing values and used this network as gold standard. Next, to simulate missing values, we generated new phospho-proteomic datasets by randomly replacing pre-defined ratios of un-imputed phosphosites with missing values (r=10%, 25%, 50%). These new matrices were then imputed using DreamAI. Finally, we applied SPHINKS to predict the kinase-phospho-substrates interaction networks on the imputed matrices, and compared them with the network reconstructed with the original dataset without missing values. The Area Under the Curve (AUC) was computed as measure of accuracy.

### Validation of the Kinase-Phospho-Substrate Interaction Predictions by SPHINKS

The reconstruction of the network using SPHINKS is based on the training of a SVM classifier using a set of experimentally validated kinase-phospho-substrates interactions available from PhosphoSitePlus to infer novel interactions. The first step for the benchmarking of SPHINKS algorithm consists in the evaluation of its performances in the prediction of the true positive kinase-phospho-substrates interactions.

To do so, we performed a 10-fold cross-validation analysis by randomly dividing the experimentally validated kinase-phospho-substrate interactions from PhosphoSitePlus into 10 subsets for training and testing the SVM classifier. At each iteration, each subset takes turn as test set, while the remaining nine subset are treated as the training set. The workflow for each fold is as follows:
i. We trained the SVM classifier using the training subset of experimentally validated kinase-substrate interactions as positive training set plus a random selection of unknown interactions as negative training set.
ii. Next, for the test set we used a subset of experimentally validated kinase-substrate interactions and a randomly selected set of unknown interactions, completely independent of the negative training set in step i., as putative negative interactions. We used the SVM classifier trained in step i. to obtain the score for the interactions in the test set, a value between 0 and 1 representing the probability for each phospho-site to be a kinase substrate.
iii. To improve prediction accuracy, we applied the bagging approach by keeping fixed the positive training set with multiple random samples of the negative training set. Step i. and ii. were repeated 100 times to derive SPHINKS scores as the average of the SVM outputs from all iterations.
iv. Finally, we compared the predicted SPHINKS scores to the experimentally validated kinase-phospho-substrate interactions from the test set and derived the Area Under the Curve (AUC) from the Receiver Operating Characteristics (ROC) curve, a measurement of the performance of classification experiments. AUC values range from 0 to 1, with 0.5 indicating uninformative classifiers, and 1 representing perfect classification.

We note that in step ii. the negative interactions are randomly selected from the set of unknown interactions, since the “true” set of negative interaction is not known. This means that some of the selected phospho-site included in the negative test set might be real substrates of a particular kinase, and as a result the actual AUC values could be underestimated.

### Validation of the Kinase Activity Estimate Inferred by SPHINKS

In order to evaluate the robustness of SPHINKS in inferring kinase activity and quantitatively estimate the dependence on the predicted kinase-substrate interactions, we randomly perturbed the SPHINKS network. The workflow is as follows:
i. From the predicted kinase-phospho-substrate interaction network, we generated a set of perturbations of interactions by replacing a pre-determined percentage of phosphor-substrates corresponding to p=bottom 5%, 10%, 15%, 20%, 50% of the SPHINKS scores with random phosphosites. This allows the evaluation of how much the different levels of mis-classifications of the kinase-phospho-substrate interactions affect the kinase activity estimate by SPHINKS.
ii. For each percentage, we constructed n=100 runs of randomly generated perturbed networks.
iii. For each percentage and run, we derived the SPHINKS kinase activity for 154 kinases and 85 samples.
iv. For each percentage and run, we derived the Δ-activity, a score representing the difference (in percentage) between the kinase activity (NES) inferred using the original unperturbed network [Act(MK)^{u}] and the activity inferred using the perturbed networks [Act(MK)^{p}], calculated as:

$$Δ Act ( MK ) = abs ( \frac{{Act ( MK )}^{u} - {Act ( MK )}^{p}}{{Act ( MK )}^{u}} ) .$$

Results are presented for each kinase as the average Δ-activity across all runs at each ratio of perturbation. In detail, we first extracted the average Δ-activity of individual kinases across all samples in each run. Next, for each kinase, we derived the average Δ-activity across all the runs at each perturbation percentage. For each run we calculated the average Δ-activity across all kinases at each ratio of perturbation. In detail, we first extracted the average Δ-activity of individual kinases across all samples in each run. Next, for each run, we derived the average Δ-activity across all kinases at each perturbation percentage.

### Comparison of the Kinase Activity Estimate Inferred by SPHINKS and Other Methods

We benchmarked SPHINKS against widely used kinase-phosphosite inference methods. We considered two recently reported approaches for substrate-based kinase activity estimation Kinase-Substrate Enrichment Analysis (KSEA)⁵⁷⁻⁵⁸ and Kinase Enrichment Analysis 3 (KEA3)⁵⁹. KSEA derives kinase activity by calculating a z-score and considering the abundance of known phospho-site substrates of a given kinase between group of interest and control group. KSEA uses two different kinase-phospho-substrate interaction networks, one that considers only the experimentally validated kinase-phospho-substrate interactions from PhosphoSitePlus, and a another that includes also predicted relationship from NetworKIN¹²⁶. The KSEA kinase activity inference derived from both networks was considered in the benchmarking. KEA3 infers upstream kinases whose putative substrates are overrepresented in a list of differentially phosphorylated proteins between group of interest and control group. The approach integrates the ranking of the kinase enrichment from 11 protein-protein and kinase-substrates interaction libraries using two different methods, MeanRank and TopRank. Both kinase enrichment score rank methods from KEA3 were considered in the benchmarking.

We benchmarked SPHINKS using two independent approaches. First, we used a dataset reporting the downstream changes in the abundance of phospho-protein after perturbations of upstream kinase by stimulators or inhibitors^{58,60,61}. This benchmark data brings together 24 studies encompassing 103 kinase-perturbation annotations (considered as “gold standard”) for 30 different kinases and 61,181 phospho-sites identified in at least one perturbation. Since usual benchmarking approaches like area under receiver operating characteristics curve and precision at recall 0.5 consider high number of predictions, which would not be practical to experimentally validate, we employed a metric defined as “top-k-hit” [Pₕᵢₜ(k)], that focuses on the top k kinase predictions, as described⁶¹. In our experiment, we use k=10. Since we are interested in comparing the different methods for kinase activity inference, in testing SPHINKS we considered only the set of experimentally validated kinase-phospho-substrates interactions from PhosphoSitePlus and SPHINKS scores.

In the second approach, we evaluated whether the GBM subtype-specific kinases uncovered by SPHINKS would be identified by the other approaches. We applied each method on the CPTAC-GBM dataset and then derived the rankings of the 129 kinases included in all 5 methods for each subtype. For SPHINKS, we ranked the kinases according to the MWW-score from the differential activity analysis of each subtype compared with all the others using MWW test. For KSEA PhosphoSitePlus and KSEA PhosphoSitePlus+NetworKIN, we ranked the kinases on the basis of the kinase's z-score for each subtype compared with all the others. For KEA3 MeanRank and KEA3 TopRank, we ranked the kinases on the basis of the MeanRank or TopRank for each subtype (considering the highest 300 proteins differentially phosphorylated in each functional subtype compared with all the others). Finally, for each kinase we derived the A-rank as the difference between the rank of the kinase inferred by SPHINKS and the rank of the same kinase inferred by any other approach. Thus, a Δ-rank lower than 0 indicates that the rank of SPHINKS is lower than the rank of the other approach, and therefore that SPHINKS returns a higher activity of the particular kinase; Δ-rank higher than 0 indicates the opposite scenario.

### Processing and Library Preparation of the In-House GBM IDH Wild Type Cohort

The cohort is composed of 178 formalin-fixed paraffin-embedded (FFPE) IDH wild type GBM samples, 45 of which had matched frozen specimens. RNA was extracted using the Maxwell® Rapid Sample Concentrator Instrument (Promega) and Maxwell® RSC simplyRNA Tissue Kit (Promega, AS1340) for frozen samples or Maxwell® RSC RNA FFPE Kit (Promega, AS1440) for FFPE specimens. RNA extracted from FFPE or frozen tissue were analyzed using the same workflow. cDNA libraries were prepared with QuantSeq 3′ mRNA-Seq Library Prep Kit FWD (Lexogen, 015)¹²⁷. In brief, libraries were prepared with oligo-dT priming, with no prior poly(A) enrichment or ribosomal RNA depletion required. After first-strand synthesis, second strand synthesis was initiated by random priming, and Illumina-specific linker sequences were introduced. The resulting double-stranded cDNA was purified with magnetic beads, and the library then amplified, introducing the sequences required for cluster generation. Illumina libraries were then multiplexed compatibly with single-end sequencing128 and sequenced on the Illumina HiSeq platform (100-bp single end). Sequencing quality was assessed through error rate and base quality distributions of reads for each sample. We filtered the raw data, removing reads containing adaptors and reads containing more than 10% of bases that could not be determined, and reads including over 50% bases with a Phred quality score≤5. Cleaned reads had error rate mean ≤2% and Q30≥90% for all samples. The reads were aligned to the human reference genome (GRCh37/hg19) using STAR and the expression was quantitated at gene level using featureCounts, a count-based estimation algorithm. Samples were finally upper-quantile normalized, according to a described pipeline¹³.

### Development of the Probabilistic Classification Tool for IDH Wild Type GBM

The goal was to design an algorithm that calculates the probability that a given GBM belongs to one of four defined functional subtypes using a limited number of features and assign the sample to a subtype based on the calculated probability. We used the Agilent expression data of 506 tumors from the TCGA GBM IDH wild type cohort that we had previously classified into one of the four functional subtypes, to train two different classifiers based on the multinomial regression model, one using RNA-Seq data obtained from frozen samples and the other specific for RNA-Seq data obtained from FFPE samples. We evaluated their performances with various indices: train classification error, cross-validation error, the Area Under the ROC (AUROC) curve, test classification error, sensitivity, specificity and precision for the subtype assignments¹⁰².

Workflow for Building the GBM Classifier Using RNA-Seq Data Obtained from Frozen Specimens

We used 506 tumors from the TCGA GBM IDH wild type cohort profiled by the Agilent gene expression microarray as training set as these tumors were previously assigned to each of the four functional subtypes based on orthogonal validation across multiple platforms¹³. The standardized expression of all the genes from the subtype-specific gene signatures was used to train a multinomial regression model with lasso penalty using glmnet R package (alpha=1, family=“multinomial”)⁹⁹⁻¹⁰¹. We applied a 10-fold cross-validation in order to select the best model with the lowest cross-validation error based on the misclassification error as loss measure. As test set (ground truth), we considered two different GBM IDH-wt RNA-Seq datasets:
a. 127 tumors from the TCGA IDH wild type GBM cohort profiled by RNA-Seq and classified according to the subtyping of the matched Agilent microarray expression tumors. The Agilent microarray expression-based classification assignments were orthogonally validated across multiple platforms including fCNVs, somatic mutations, DNA methylation and miRNA gene signatures¹³ and was used as ground truth.
b. 85 tumors from the CPTAC IDH wild type GBM cohort profiled by RNA-Seq and classified according to one of the four functional subtypes as described herein. Having provided multi-omics orthogonal cross-validation for the CNV+RNAseq-based classification of these 85 IDH wild type GBM (global proteomics, phospho-proteomics, lipidomics, metabolomics and acetylomics, described herein), the CNV+RNAseq-based assignments of the 85 IDH wild type GBM in the CPTAC cohort was used as ground truth.

We classified the test samples into one of the four functional subtypes if the fitted probability of a particular subtype was the highest and the sample showed a simplicity score above 0.35. The simplicity score for each individual tumor was computed as the difference between the highest fitted probability (dominant subtype) and the mean of the other subtypes (non-dominant), thus representing the subtype activation (higher scores indicate lower transcriptional complexity and lower scores multi-subtype activation). Tumors with simplicity score below the selected cutoff were unclassified. Using this threshold, we classified 80% of the TCGA GBM cohort and 79% of the CPTAC-GBM cohort.

Workflow for building a GBM classifier using RNA-Seq data obtained from FFPE specimens.

For the FFPE model, we used a similar approach as for samples profiled by RNA-Seq data from frozen tissue, with some modifications. We generated RNA-Seq data from FFPE tissue of 178 GBM IDH wild type, 45 of which were also independently sequenced from matched frozen specimens. To retain in the FFPE expression matrix only those genes having an expression profile consistent with the corresponding frozen specimens, we calculated the correlation of the expression of each gene between the 45 frozen and the 45 FFPE samples and retained only genes with a Spearman correlation value above 0.22 (4,673 genes). Independently, we classified the 45 fresh frozen samples-extracted RNA to each individual subtype on the basis of the highest Normalized Enrichment Score (NES) by MWW-GST using the functional subtypes signatures¹³. Using the classification of the 45 frozen samples as “gold standard”, we derived FFPE-specific signatures for each subtype comprising the 50 highest genes from the ranked list that was generated using MWW test on the FFPE expression matrix, as described¹³. We used 506 tumors from the TCGA IDH wild type GBM profiled by the Agilent microarray platform and classified into one of the four functional subtypes as training set, and the standardized expression of all the genes from the FFPE-specific signatures to train a multinomial regression model with lasso penalty using glmnet R package (alpha=1, family=“multinomial”). We applied a 10-fold cross-validation in order to select the best model with the lowest cross-validation error based on the misclassification error as loss measure. The remaining 133 samples that lacked RNA-Seq data from frozen specimens and had not been used to define the FFPE-specific signatures were classified into one of the 4 functional subtypes if the fitted probability of a particular subtype was the highest and the sample showed a simplicity score above 0.25. Using this threshold, we classified 73% of the 133 FFPE tumors. In order to assess the stability and accuracy of the FFPE model, we performed an independent analysis to obtain an unbiased assignment of the FFPE samples. The expression of the genes from the FFPE-specific signatures were used to inform a consensus clustering on the Euclidean distance matrix of the 178 FFPE-derived RNA-Seq (10,000 random samplings using 70% of the samples and the Ward linkage method, with k=4 1750 clusters). We then labeled all the samples by assigning each individual cluster derived from consensus cluster to each functional subtype using the classification of the 45 matched frozen samples as “anchors”. We found a 91% concordance in the classification of 45 matched frozen and FFPE-derived RNA-Seq (41 out of 45). Finally, the unbiased label assignments of the 133 unmatched FFPE samples were used to evaluate the prediction abilities of the classifier.

Association of the Four Functional Subtypes with Clinical Features in GBM

Clinical data for TCGA-GBM patients were downloaded using TCGAbiolinks package available on R Bioconductor. Demographic characteristics including age, gender and pathway-based assignment were available for 503 GBM tumors. Patients were segregated in three distinct age categories: [10-40 years], [40-65 years] and >65 years. Quantification of radiomic features, including volumetric-based (volume enhancing/non-enhancing over tumor core, volume edema over whole tumor) and spacial-based (tumor location) features, were available for 88 pre-operative multimodal MRI of TCGA-GBM from the Cancer Imaging Archive (TCIA). For each tumor location considered (parietal, frontal, and temporal), patients were segregated in high or low group when the percentage of the tumor detected in the location was higher/lower than 50%, respectively. The univariate logistic regression analysis was performed to assess the association between demographic or radiomic features and functional subtypes or the combination of metabolic/neurodevelopmental axis.

Radiologist-made assessments (proportion of necrosis and proportion of edema) of GBM MRI features from TCGA (n=63 GBM tumors with available pathway-based subtype classification) have been previously published²². Evaluation of proportion of deep white matter invasion available through TCIA was obtained by the integration of data from²³ and REMBRANDT for a total of 54 GBM.

Association of the four functional subtypes with intrinsic imaging features in GBM 175 quantitative radiomic features from 88 pre-operative multimodal MRI of TCGA-GBM were selected from TCIA as described¹²⁹. To compare each subtype versus all the others, we performed a differential using the Wilcoxon test (fold-change>0.3 and p<0.05). The association between pathway-based subtypes and subgroups identified by the unsupervised clustering was assessed by the χ2 test.

### Cell Culture

Human cell lines are: HEK293T (ATCC CRL-11268). Cells were cultured in DMEM supplemented with 10% fetal bovine serum (FBS, Sigma). Cells were transfected using Lipofectamine 2000 (Invitrogen) or the calcium phosphate method. Lentiviral infection was performed as previously described⁵⁶. shRNA sequences for PKCδ are:

PRKCD shRNA 1 (TRCN0000010193):
(SEQ ID NO.: 1)
CCGGGGCCGCTTTGAACTCTACCGTCTCGAGACGGTAGAGT
TCAAAGCGGCCTTTTT;
PRKCD-shRNA 2 (TRCN0000379731):
(SEQ ID NO.: 2)
GTACCGGCATTACTTGAATGTAGTTATCCTCGAGGATAACT
ACATTCAAGTAATGTTTTTTG;

Patient-derived organoids (PDOs) were obtained using excess material collected for clinical purposes from de-identified brain tumor specimens. Donors (patients diagnosed with GBM) were anonymous. Work with these materials was designated as IRB exempt under paragraph 4 and it is covered under IRB protocol #IRB-AAAI7305 and Onconeurotek tumor bank certification (NF S96 900) and authorization from Ethics committee (CPP Ile de France VI, ref A39II), and the French Ministry for research (AC 2013-1962). PDOs were grown in DMEM:F12 containing 1×N2 and B27 supplements (Invitrogen) and human recombinant FGF-2 and EGF (20 ng/ml each; Peprotech). Cells were routinely tested for mycoplasma contamination using the Mycoplasma Plus PCR Primer Set (Agilent Technologies) and were found to be negative. Cell authentication was performed using short tandem repeats (STR) at the ATCC facility.

Cell growth and clonogenic assay. Time course analysis of the cellular growth of shPRKCD infected PDOs or the empty vector was performed by plating 4,500 cells per well in 96-well plates. Viability was determined using CellTiterGlo assay reagent (Promega, G7570) and the GloMax-Multi+ Microplate Multimode Reader (Promega). Data are mean±s.d. of five replicates, and experiments were repeated twice. For clonogenic assay of PDO cells treated with increasing concentration of BJE6-106, 1,500 cells were plated in 6-well plates in triplicate. Cells were fixed in methanol and stained with crystal violet 2 weeks later. Colonies with more than 50 cells were scored. Data are mean±s.d. of triplicate observations from one representative experiment. Experiments were repeated twice.

### Intracellular Glucose Uptake and Triacylgliceride Accumulation

Measurement of the rate of glucose uptake and triacylgliceride accumulation in shPRKCD and control infected GPM PDO cells were performed using Glucose Uptake-Glo™ Assay (Promega, J1342) and Triglyceride-Glo™ Assay (Promega, J3161), respectively according to the manufacturer instructions. Briefly, cells were plated at density of >1,000 cells in 130 μl (3 replicates) of medium containing 8 mM of glucose and 2 mM of glutamine in opaque white 96-well plate. Glucose uptake was assayed 24 hrs later and triacylglicerides were measured 48 hrs after plating. Luminescence was recorded with a 0.3 seconds integration on GloMax-Multi+ Microplate Multimode Reader (Promega) instrument. Data are mean±s.d. of 3-6 observations from two independent experiment.

### Compound and Irradiation-Drug Combination Treatment of PDOs

Cells were cultured in DMEM/F12 medium supplemented with N-2, B-27, EGF and FGF. Cells were plated in 130 μl in opaque white 96-well plates. Twenty-four hrs later cells were treated with 3-fold serial dilutions of compounds as indicated in six replicates for 72 hrs. Viability was determined using CellTiterGlo assay reagent (Promega, G7570) and GloMax-Multi+ Microplate Multimode Reader (Promega). For the irradiation-drug combination treatment, PDOs cells were plated in 96 well plates 24 hrs before treatment. Cells were treated with 3-fold serial dilutions of M3814 two hrs before exposure to various irradiation doses (2, 4, 8 Gy at 0.7 Gy/min) from a ¹³⁷Cs source (GammaCell 40 irradiator, Teratronics). Mock irradiated cells were cultured in parallel. Viability was determined 96 hrs later using CellTiterGlo assay reagent (Promega, G7570) and GloMax-Multi+ Microplate Multimode Reader (Promega). Data are mean±s.d. of the viability ratio from 6 observations in 8 PPR and 8 GPM PDOs. Experiments were performed at least twice with similar results. Clonogenic assays for the evaluation of irradiation-drug combination were performed in 96 well plates. Each experimental point was plated in 3 independent 96 well plates. The number of wells containing PDO spheres were scored and the value of control untreated cell was considered 100% clonogenicity for the specific PDO. Experiments were repeated at least twice.

### Immunofluorescence Analysis of γH2AX Foci

Cells were fixed with 4% paraformaldehyde, permeabilized with cold methanol for 90 seconds at 4° C., and blocked with 5% BSA, 0.05% Triton X-100 in PBS for 30 minutes. Cells were exposed to primary antibody phospho-H2AX 1:500 (S139, CST, #2577) for one hour at room temperature followed by Cy3-conjugated anti rabbit (Invitrogen, A10520) for one hour at room temperature. Nuclei were stained with DAPI (Sigma). Images were acquired using a Nikon Ti Eclipse inverted microscope for spinning-disk confocal microscopy equipped with a Plan Apochromat 60× oil/1.4 NA DIC objective. Counting of TH2AX foci in individual nuclei were analyzed by ImageJ (NIH, Bethesda, USA) by specific in-built find Maxima>Prominence>Point Selection plug-in. Minimum 50 nuclei in at least ten representative images were included for analysis in each treatment group.

### Western Blot

Cells were lysed in RIPA buffer (50 mM Tris-HCl pH 7.5, 150 mM NaCl, 1 mM EDTA, 1% NP40, 0.5% sodium dexoycholate, 0.1% sodium dodecyl sulfate, 1.5 mM Na₃VO₄, 50 mM sodiumfluoride, 10 mM sodium pyrophosphate, 10 mM β-glycerolphosphate and EDTA-free protease inhibitor cocktail; Roche). Lysates briefly sonicated, cleared by centrifugation at 15,000 r.p.m. for 15 min at 4° C., separated by SDS-polyacrylamide gel electrophoresis and transferred to polyvinylidene difluoride membrane. Membranes were blocked in Tris-buffered saline with 5% nonfat milk and 0.1% Tween 20 and probed with primary antibodies overnight at 4° C. Antibodies0 and concentrations were as follows: p-DNA-PK 1:1,000 (Ser2056, CST, #68716), DNA-PK 1:1,000 (CST, #38168), p-NBS1 1:1,000 (Ser343, CST, #3001), NBS1 1:1,000 (CST, #81234), p-KAP1 1:1,000 (Ser824, Abcam, ab133440), KAP1 1:1,000 (Abcam, ab109287), p-CHK1 (Ser317, CST, #12302), CHK1 (CST, #2360), p-PKCδ 1:1,000 (Tyr311, CST, #20555), PKCδ 1:1,000 (CST, #96165), p-STAT3 1:1,000 (Tyr705, CST, #91455), STAT3 1:1,000 (CST, #49045) p-AKT 1:1,000 (Ser473, CST, #40605), p-AKT 1:1,000 (Thr308, CST, #13038S), AKT 1:1,000 (CST, #49615), p-ERK1/2 1:1,000 (Thr202/Tyr204, CST, #4370), ERK1/2 1:1,000 (CST, #9102), GAPDH 1:10,000 (Abcam, ab9484), Vinculin 1:10,000 (Sigma, no. V9131), and j-actin 1:10,000 (Sigma, no. A5441). Secondary antibody anti-mouse or anti-rabbit conjugated horseradish peroxidase was purchased from Invitrogen (no. 31438 and no. 31458, respectively), and either Enhanced ChemiLuminescence (Amersham, no. RPN2209) or Super Signal West Femto (Thermo Scientific, no. 34095) was used for detection.

### Statistics and Reproducibility

In general, at least two independent experiments were performed, with a minimum n number of three biological replicates as specified in figure legends. No statistical methods were used to predetermine sample size. No data were excluded from the analyses; the experiments were not randomized; the Investigators were not blinded to allocation during experiments and outcome assessment. Comparisons between two groups were analyzed by the Welch t-test (two-tailed, un-equal variance) or the Mann-Whitney-Wilcoxon test. Comparison between three or more groups were assessed by analysis of variance (ANOVA). Results in graphs are expressed as means±s.d. or means±s.e.m. as presented in figure legends for the indicated number of observations. Box plots span the first to third quartiles and whiskers show the 1.5× interquartile range. All statistical analyses were performed and p-values were obtained using the GraphPad Prism software 8.0.

### Data and Code Availability

RNA-Seq expression data of the 178 FFPE-derived and 45 frozen GBM IDH-wt tumors have been submitted to Synapse (http://synapse.org, accession no. syn27042663). The source code used for the SPHINKS approach together with the final GBM-specific kinome phosphorylome network are available at GitHub: github.com/miccec/MAKINA. The Shiny app of the frozen and FFPE classification tools is available at lucgar88.shinyapps.io/GBMclassifier.

### REFERENCES

109 Berger, A. C. et al. A Comprehensive Pan-Cancer Molecular Study of Gynecologic and Breast Cancers. Cancer Cell 33, 690-705 e699 (2018).
110 Cancer Genome Atlas, N. Comprehensive molecular portraits of human breast tumours. Nature 490, 61-70 (2012).
111 Cancer Genome Atlas Research, N. Comprehensive genomic characterization of squamous cell lung cancers. Nature 489, 519-525 (2012).
112 Mukherjee, A. et al. Associations between genomic stratification of breast cancer and centrally reviewed tumour pathology in the METABRIC cohort. NPJ Breast Cancer 4, 5 (2018).
113 Curtis, C. et al. The genomic and transcriptomic architecture of 2,000 breast tumours reveals novel subgroups. Nature 486, 346-352 (2012).
114 Aryee, M. J. et al. Minfi: a flexible and comprehensive Bioconductor package for the analysis of Infinium DNA methylation microarrays. Bioinformatics 30, 1363-1369 (2014).
115 Saghafinia, S., Mina, M., Riggi, N., Hanahan, D. & Ciriello, G. Pan-Cancer Landscape of Aberrant DNA Methylation across Human Tumors. Cell Rep 25, 1066-1080 e1068 (2018).
116 Stoney, R. A., Schwartz, J. M., Robertson, D. L. & Nenadic, G. Using set theory to reduce redundancy in pathway sets. BMC Bioinformatics 19, 386 (2018).
117 Kramer, A., Green, J., Pollard, J., Jr. & Tugendreich, S. Causal analysis approaches in Ingenuity Pathway Analysis. Bioinformatics 30, 523-530 (2014).
118 Fabregat, A. et al. Reactome graph database: Efficient access to complex pathway data. PLoS Comput Biol 14, e1005968 (2018).
119 Binder, J. X. et al. COMPARTMENTS: unification and visualization of protein subcellular localization evidence. Database (Oxford) 2014, bau012 (2014).
120 Mordelet, F. & Vert, J. P. Supervised inference of gene regulatory networks from positive and unlabeled examples. Methods Mol Biol 939, 47-58 (2013).
121 Cerulo, L., Elkan, C. & Ceccarelli, M. Learning gene regulatory networks from only positive and unlabeled data. BMC Bioinformatics 11, 228 (2010).
122 Hornbeck, P. V. et al. PhosphoSitePlus, 2014: mutations, PTMs and recalibrations. Nucleic Acids Res 43, D512-520 (2015).
123 He, H. B. & Garcia, E. A. Learning from Imbalanced Data. Ieee T Knowl Data En 21, 1263-1284 (2009).
124 Breiman, L. Bagging predictors. Mach Learn 24, 123-140 (1996).
125 Eid, S., Turk, S., Volkamer, A., Rippmann, F. & Fulle, S. KinMap: a web-based tool for interactive navigation through human kinome data. BMC Bioinformatics 18, 16 (2017).
126 Linding, R. et al. Systematic discovery of in vivo phosphorylation networks. Cell 129, 1415-1426 (2007).
127 Jang, J. S. et al. Application of the 3′ mRNA-Seq using unique molecular identifiers in highly degraded RNA derived from formalin-fixed, paraffin-embedded tissue. BMC Genomics 22, 759 (2021).
128 Corley, S. M., Troy, N. M., Bosco, A. & Wilkins, M. R. QuantSeq. 3′ Sequencing combined with Salmon provides a fast, reliable approach for high throughput RNA expression analysis. Sci Rep 9, 18895 (2019).
129 Rathore, S. et al. Radiomic MRI signature reveals three distinct subtypes of glioblastoma with different clinical and molecular characteristics, offering prognostic value beyond IDH1. Sci Rep 8, 5087 (2018).

What is claimed is:

1. A method of treating cancer in a subject in need thereof, the method comprising:
analyzing a kinome and a phosphorylome from proteomic-phosphoproteomics data from cells of a tumor sample from the subject;
identifying a master kinase from the kinome and/or phosphorylome analysis; and
administering to the subject a therapeutically effective amount of a pharmaceutical composition, wherein the composition modulates the master kinase.

2. The method of claim 1, wherein the cancer is a glioma, a pediatric glioma, a glioblastoma (GBM), an IDH wild-type GBM, a breast cancer, or a lung squamous cell carcinoma.

3. The method of claim 1, wherein the tumor sample comprises a tissue sample.

4. The method of claim 3, wherein the tissue sample is a frozen tissue sample or is embedded in paraffin.

5. The method of claim 1, wherein the method further comprises classifying the cancer into a tumor subtype.

6. The method of claim 5, wherein the tumor subtype is a glycolytic/plurimetabolic (GPM) subtype, mitochondrial (MTC) subtype, neuronal (NEU) subtype, or proliferative/progenitor (PPR) subtype.

7. The method of claim 1, further comprising experimentally validating the master kinase.

8. The method of claim 1, wherein identifying the master kinase comprises:
(i) training a support vector machine (SVM) classifier with a positive data set comprising a set of known substrates of a specific kinase and a negative data set comprising a subset of randomly selected unknown interactions using kinase abundance from proteomics and substrate abundance from proteomic-phosphoproteomics data of the sample;
(ii) computing a probability score for all the kinase-substrate pairs in the network according to the SVM classifier;
(iii) repeating steps (i) and (ii) with the same positive data set and a different negative data set;
(iv) performing machine learning ensemble meta-algorithm bagging to obtain an average of scores from each iteration of steps (i) and (iv);
(v) defining a list of predicted kinase-substrate interactions by selecting a threshold for the average SVM score and retaining only interactions whose average score was above the selected threshold and whose Spearman correlation between protein kinase global abundance and substrate phospho-site abundance was positive; and
(vi) calculating master kinase activity as the difference of the weighted average of the predicted substrate's abundances using the SVM score of kinase-substrate interactions as weight and the weighted average of randomly selected control substrate-set.

9. The method of claim 8, wherein the selected threshold for the average SVM score is greater than 50% of the known interactions.

10. The method of claim 8, wherein the set of known substrates of a specific kinase comprises validated kinase-substrate interactions.

11. The method of claim 8, wherein steps (i) and (ii) are repeated around 100 times.

12. The method of claim 8, wherein in step (v), kinases with less than 10 interactions are removed from the list.

13. The method of claim 5, wherein classifying the cancer into a tumor subtype comprises a multi-omics approach comprising analyzing kinase activity, radiomics, copy number variants (CNV), single nucleotide variants (SNV), and a gene expression profile of cells of the tumor sample.

14. The method of claim 13, wherein the cancer is classified as a mitochondrial (MTC) subtype if it is associated with a plurality of high CET, low NET, enhanced PHKG2 expression, SLC45A1 del, RERE del, 1p36 del, enhanced OXPHOS activity, enhanced TCA cycle activity, and enhanced mitochondrial translation.

15. The method of claim 13, wherein the cancer is classified as a glycolytic/plurimetabolic (GPM) subtype if it is associated with a plurality of high CET, low NET, high edema, male demographic, 40-65 years demographic, MET amp, NF1 mut/del, enhanced PKCδ, P38D, or MK-2 expression, enhanced glycolysis, enhanced lipid storage, or hypoxia.

16. The method of claim 15, further comprising administering to the subject a therapeutically effective amount of a pharmaceutical composition comprising BJE-10676.

17. The method of claim 13, wherein the cancer is classified as a neuronal (NEU) subtype if it is associated with a plurality of low CET, high NET, high WM invasion, low necrosis, ATRX mut, TCGA, enhanced GSK3β, PCKε, or PAK1/3 expression, enhanced neuronal differentiation, or excitatory synapses.

18. The method of claim 13, wherein the cancer is classified as a proliferative/progenitor (PPR) subtype if it is associated with a plurality of low CET, high NET, low WM invasion, high edema, EGFR amp, CDK6 amp, enhanced DNA-PKcs, CDK1/2/6, or CHK2 activity, enhanced cell cycle activity, enhanced DNA replication, or enhanced DDR pathway activation.

19. The method of claim 18, further comprising administering to the subject a therapeutically effective amount of a pharmaceutical composition comprising M3814 (nedisertib).

20. The method of claim 1, wherein the composition comprises an inhibitory RNA.

21. The method of claim 20, wherein the inhibitory RNA is one or more of a miRNA, a siRNA, a shRNA, or a piRNA.

22. The method of claim 5, wherein classifying the cancer into a tumor subtype comprises using a probabilistic classifying method comprising:
obtaining a gene expression profile of the tumor sample;
comparing the gene expression profile of the tumor sample with a gene expression profile of a set of tumors with known tumor subtypes; and
correlating the gene expression profile with the best fitting tumor subtype.

23. The method of claim 22, wherein the tumor sample is classified into a tumor subtype if the difference between the correlation with the tumor subtype and other tumor subtypes is above a threshold value.

24. The method of claim 23, wherein the threshold value is in the form of a simplicity score, wherein the simplicity score is a different between a highest fitted probability (dominant subtype) and a mean of the other subtypes (non-dominant).

25. The method of claim 24, wherein the threshold value is a simplicity score of 0.35.

26. The method of claim 1, wherein the master kinase is a phosphatidylinositol 3-kinase related kinase, (Protein Kinase C delta) PKCδ, or (DNA-dependent protein kinase catalytic subunit) DNA-PKcs.

27. The method of claim 1, wherein the composition is administered in combination with treating the subject with ionizing radiation (IR).


SEQUENCE LISTING (2 sequences)

SEQ ID NO: 1 - DNA, 57 bp, synthetic construct
  source 1..57: mol_type = other DNA
ccggggccgctttgaactctaccgtctcgagacggtagagttcaaagcggccttttt

SEQ ID NO: 2 - DNA, 62 bp, synthetic construct
  source 1..62: mol_type = other DNA
gtaccggcattacttgaatgtagttatcctcgaggataactacattcaagtaatgttttttg